(19) United States
(12) Patent Application Publication
(10) Pub. No.: US 2024/0050476 A1
(43) Pub. Date: Feb. 15, 2024

(54) COMBINATION OF DEOXYRIBONUCLEASE ENZYME AND CELL THERAPIES FOR TREATMENT OF CANCER

(71) Applicant: CLS THERAPEUTICS LIMITED, St. Peter Port, Guernsey (GG)

(72) Inventors: Dmitry Dmitrievich GENKIN, Milan (IT); Georgy Viktorovich TETS, New York, NY (US); Viktor Veniaminovich TETS, New York, NY (US); Alexey Vyacheslavovich STEPANOV, Moscow (RU)

(73) Assignee: CLS THERAPEUTICS LIMITED, St. Peter Port, Guernsey (GG)

(21) Appl. No.: 18/271,453

(22) PCT Filed: Jan. 7, 2022
(86) PCT No.: PCT/US2022/011648

§ 371 Date: Jul. 8, 2023

Related U.S. Application Data
(60) Provisional application No. 63/135,438, filed on Jan. 8, 2021.
(60) Provisional application No. 63/148,304, filed on Feb. 11, 2021.

(51) Int. Cl. A61K 35/17; A61K 38/46; C12N 9/22; A61K 48/00; C12N 15/86; A61K 39/00; A61K 9/00; A61P 35/00; C07K 16/28

(52) U.S. Cl. CPC A61K 35/17; A61K 38/465; C12N 9/22; A61K 48/005; C12N 15/86; A61K 39/4611; A61K 39/4631; A61K 39/464454; A61K 9/0019; A61P 35/00; C07K 16/2803; A61K 39/464412; C12Y 301/21001; A61K 2239/13

(57) ABSTRACT

The invention relates to methods for treatment of cancers utilizing a combination of a deoxyribonuclease enzyme and adoptive cell immunotherapy comprising cells expressing a chimeric antigen receptor (CAR) or a T cell receptor (TCR). In particular embodiments, the deoxyribonuclease enzyme can be given parenterally or encoded by a vector or secreted by a cell comprising TCR or CAR.

### CROSS-REFERENCE TO RELATED APPLICATIONS

This application claims priority to U.S. Provisional Patent Application No. 63/135,438, filed Jan. 8, 2021, and U.S. Provisional Patent Application No. 63/148,304, filed Feb. 11, 2021, the disclosures of which are herein incorporated by reference in their entirety.

### SEQUENCE LISTING

The instant application contains a Sequence Listing which has been submitted electronically in ASCII format and is hereby incorporated by reference in its entirety. Said ASCII copy, created on Jan. 7, 2022, is named 252732_000023_SL.txt and is 117,776 bytes in size.

### FIELD OF THE INVENTION

The invention relates to methods for treatment of cancers utilizing a combination of a deoxyribonuclease enzyme and adoptive cell immunotherapy comprising cells expressing a chimeric antigen receptor (CAR) or a T cell receptor (TCR).

### SUMMARY OF THE INVENTION

The present invention addresses great need in the art for new and more effective treatments of cancer.

In one aspect, the invention provides a method of treating a cancer in a subject in need thereof, comprising administering to the subject a deoxyribonuclease (DNase) enzyme and at least one cell comprising a chimeric antigen receptor (CAR) or a T cell receptor (TCR). In some embodiments, said DNase enzyme is used in an amount effective to reduce expression of immunosuppressive proteins in tumor tissue and/or increase content of CAR-expressing cells or TCR-expressing cells within tumor tissue.

In another aspect, the invention provides a method of preventing one or more side effects of a CAR-T cell therapy in a subject in need thereof, comprising administering to the subject a deoxyribonuclease (DNase) enzyme, wherein said DNase enzyme is administered simultaneously or sequentially with said CAR-T cell therapy. In some embodiments, the side effects of the CAR-T cell therapy are selected from neurologic side effects, cytokine release syndrome (CRS), neutropenia, anemia, pyrexia, febrile neutropenia, and thrombocytopenia.

In a further aspect, the invention provides a method of preventing relapses of a disease following treatment of said disease by a CAR-T cell therapy in a subject, comprising administering to the subject a deoxyribonuclease (DNase) enzyme, wherein said DNase enzyme is administered simultaneously or sequentially with said CAR-T cell therapy.

In yet another aspect, the invention provides a method of increasing persistence of CAR-T cells in blood of a subject undergoing a CAR-T cell therapy, comprising administering to the subject a deoxyribonuclease (DNase) enzyme, wherein said DNase enzyme is administered simultaneously or sequentially with said CAR-T cell therapy.

In a further aspect, the invention provides a method of increasing efficacy of a CAR-T cell therapy in a subject in need thereof, comprising administering to the subject a deoxyribonuclease (DNase) enzyme, wherein said DNase enzyme is administered simultaneously or sequentially with said CAR-T cell therapy.

In some embodiments of any of the above methods, the DNase enzyme is administered as a DNase enzyme protein.

In some embodiments of any of the above methods, the DNase enzyme is encoded by a vector. In some embodiments, the vector is a gene therapy vector. In some embodiments, the vector is a recombinant adeno-associated virus (rAAV) vector comprising (i) a capsid protein and (ii) a nucleic acid comprising a promoter operably linked to a nucleotide sequence encoding the DNase enzyme. In some embodiments, the promoter is a liver-specific promoter. In some embodiments, the promoter is specific for tumor originator tissue or metastasis target tissue.

In some embodiments of any of the above methods, the DNase enzyme is encoded by a mRNA molecule.

In some embodiments of any of the above methods, the DNase enzyme is administered parenterally or to the site of the tumor.

In some embodiments of any of the above methods, the DNase enzyme is co-expressed by the cell comprising the chimeric antigen receptor (CAR) or the T cell receptor (TCR).

In some embodiments of any of the above methods, the CAR expressing cell or the TCR expressing cell is administered parenterally or to the site of the tumor. In some embodiments of any of the above methods, the CAR expressing cell or TCR expressing cell is single-target or multi-target.

In some embodiments of any of the above methods, the CAR comprises an antigen binding domain capable of specific binding to one or more antigens selected from (i) a tumor antigen selected from CD5, CD7, CD19, CD28, mesothelin, CD123, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, IL-11Ra, PSCA, PRSS21, VEGFR2, LewisY, CD24, PDGFR-beta, SSEA-4, CD20, Folate receptor alpha, FR-1, c-MET, EGFR/CD133, IL13Ra2, HER2, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Plysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, 0Y-TES1, LCK, AKAP-4, SSX2, RAGE-1, human telomerase reverse transcriptase, RU1, RU2, intestinal carboxyl esterase, and mut hsp70-2; (ii) an antigen associated with a solid tumor; (iii) a solid tumor associated antigen selected from mesothelin, EGFRvIII, GD2, CLDN6, Tn Ag, PSMA, CD97, TAG72, CD44v6, CEA, EPCAM, KIT, IL-13Ra2, leguman, CD171, PSCA, TARP, MAD-CT-1, Lewis Y, CD24, folate receptor alpha, folate receptor beta, ERBBs, MUC1, EGFR, NCAM, PDGFR-beta, MAD-CT-2, Fos-related antigen, SSEA-4, neutrophil elastase, CAIX, HPV E6 E7, ML-IAP, NA17, ALK, androgen receptor plsialic acid, TRP-2, CYP1B1, PLAC1, GloboH, NY-BR-1, sperm protein 17, HMWMAA, beta human chorionic gonadotropin, AFP, thyroglobulin, RAGE-1, MN-CA IX, human telomerase reverse transcriptase, intestinal carboxyl esterase, and mut hsp 70-2; (iv) a solid tumor associated antigen present in/on a mesothelioma, a lung cancer, a pancreatic cancer, an esophageal adenocarcinoma, an ovarian cancer, a breast cancer, a colorectal cancer, a bladder cancer, or any combination thereof; (v) a tumor antigen that is associated with a hematological cancer; (vi) a tumor antigen present in a disease selected from acute leukemias (including, without limitation, B-cell acute lymphoid leukemia (“BALL”), T-cell acute lymphoid leukemia (“TALL”), acute lymphoid leukemia (ALL)), and chronic leukemias (including, without limitation, chronic myelogenous leukemia (CIVIL) and chronic lymphoid leukemia (CLL)), and (vii) a tumor antigen present in a therapy resistant cancer, including but not limited to myeloperoxidase and neutrophil elastase, which include any protein or peptide which is a non-material variant, mutation, fragment or derivative of ScFv, Fab, CDR Loops, CDR grafts, dAb and nanobody fragments, whether produced by a cell line, expression by a hybridoma, expression in vitro by bacteria, yeast or other host cell, or any other method of synthesis.

In some embodiments of any of the above methods, the DNase enzyme comprises an amino acid sequence having at least 90% sequence identity to SEQ ID NO: 4.

In some embodiments of any of the above methods, the DNase enzyme comprises an amino acid sequence having at least 90% sequence identity to SEQ ID NO: 53.

In some embodiments of any of the above methods using a DNase enzyme protein, the DNase enzyme protein is injected intravenously once the same day or the day before the administration of the CAR expressing cells or TCR expressing cells.

In some embodiments of any of the above methods using a DNase enzyme protein, the DNase enzyme protein is injected intravenously once a week starting after the administration of the CAR expressing cells or TCR expressing cells.

In some embodiments of any of the above methods using a DNase enzyme protein, the DNase enzyme protein is injected intravenously once a month starting after the administration of the CAR expressing cells or TCR expressing cells.

In some embodiments of any of the above methods using a DNase enzyme protein, the DNase enzyme protein is injected intravenously a week before the administration of the CAR expressing cells or TCR expressing cells.

In some embodiments of any of the above methods using a DNase enzyme protein, the DNase enzyme protein is injected intravenously a month before the administration of the CAR expressing cells or TCR expressing cells.

In some embodiments of any of the above methods using a DNase enzyme protein, the DNase enzyme protein is injected intravenously once from 21 days before to 14 days after the administration of the CAR expressing cells or TCR expressing cells.

In some embodiments of any of the above methods using a DNase enzyme protein, the DNase enzyme protein is injected intravenously for at least 14 days following the administration of the CAR expressing cells or TCR expressing cells.

In some embodiments of any of the above methods using a DNase enzyme protein, the DNase enzyme protein is injected intravenously for at least 16 days following the administration of the CAR expressing cells or TCR expressing cells.

In some embodiments of any of the above methods using a DNase enzyme protein, the DNase enzyme protein is injected intravenously for at least 3 days prior, together or following the administration of the CAR expressing cells or TCR expressing cells.

In some embodiments of any of the above methods using a DNase enzyme protein, the DNase enzyme protein is injected intravenously for at least 7 days following the administration of the CAR expressing cells or TCR expressing cells.

In some embodiments of any of the above methods using a DNase enzyme protein, the DNase enzyme protein is injected intravenously for at least 7 days prior to the administration of CAR expressing cells or TCR expressing cells.

In some embodiments of any of the above methods using a DNase enzyme protein, the DNase enzyme protein is injected intravenously for at least 3 days prior or following the administration of CAR expressing cells or TCR expressing cells.

In some embodiments of any of the above methods using a DNase enzyme protein, the DNase enzyme protein is injected intravenously at a dose of at least 250 μg/kg/day.

In some embodiments of any of the above methods using a DNase enzyme protein, the DNase enzyme protein is injected intravenously at a dose of at least 600 μg/kg/day.

In some embodiments of any of the above methods using a DNase enzyme protein, the DNase enzyme protein is injected intravenously at a dose of at least 900 μg/kg/day.

In some embodiments of any of the above methods using a DNase enzyme protein, the DNase enzyme protein is injected intravenously at a dose of at least 2.5 mg/kg/day.

In some embodiments of any of the above methods using a DNase enzyme protein, the DNase enzyme protein is injected intravenously at a dose of at least 5 mg/kg/day.

In some embodiments of any of the above methods using a DNase enzyme protein, the DNase enzyme protein is injected intravenously for at least 7.5 mg/kg/day.

In some embodiments of any of the above methods, the CAR expressing cell or TCR expressing cell is further modified to express an immune checkpoint inhibitor molecule.

In some embodiments of any of the above methods, the DNase enzyme is used in an amount to increase the efficacy of CAR expressing cells in hypoxia environment.

In some embodiments of any of the above methods using a vector encoding DNase enzyme, the vector encoding DNase enzyme is injected intravenously and/or at the tumor site at least 14 days prior to the administration of the CAR expressing cells or TCR expressing cells.

In some embodiments of any of the above methods using a vector encoding DNase enzyme, the vector encoding DNase enzyme is injected intravenously and/or at the tumor site at least 3 days prior to the administration of the CAR expressing cells or TCR expressing cells.

In some embodiments of any of the above methods using a vector encoding DNase enzyme, the vector encoding DNase enzyme is injected intravenously and/or at the tumor site simultaneously with the administration of CAR expressing cells or TCR expressing cells.

In some embodiments of any of the above methods using a vector encoding DNase enzyme, the vector encoding DNase enzyme is injected intravenously and/or at the tumor site 3 days after the administration of CAR expressing cells or TCR expressing cells.

In some embodiments of any of the above methods, the CAR expressing cells are CART cells.

In a separate aspect, the invention provides a cell expressing (i) a deoxyribonuclease (DNase) enzyme and (ii) a chimeric antigen receptor (CAR) and/or a T cell receptor (TCR). In some embodiments, the cell further expresses an immune checkpoint inhibitor molecule. In some embodiments, the CAR or TCR is single-target or multi-target. In some embodiments, the CAR comprises an antigen binding domain capable of specific binding to one or more antigens selected from (i) a tumor antigen selected from CD5,CD7,CD19, CD28, mesothelin, CD123, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, IL-11Ra, PSCA, PRSS21, VEGFR2, LewisY, CD24, PDGFR-beta, SSEA-4, CD20, Folate receptor alpha, FR-1, c-MET, EGFR/CD133, IL13Ra2, HER2, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Plysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-Al, legumain, HPV E6,E7, MAGE Al, ETV6-AML, sperm protein 17, XAGE1, Tie 2, MAD-CT-1, MAD-CT-2, Fos-related antigen 1, p53, p53 mutant, prostein, survivin, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, 0Y-TES1, LCK, AKAP-4, SSX2, RAGE-1, human telomerase reverse transcriptase, RU1, RU2, intestinal carboxyl esterase, and mut hsp70-2; (ii) an antigen associated with a solid tumor; (iii) a solid tumor associated antigen selected from mesothelin, EGFRvIII, GD2, CLDN6, Tn Ag, PSMA, CD97, TAG72, CD44v6, CEA, EPCAM, KIT, IL-13Ra2, leguman, CD171, PSCA, TARP, MAD-CT-1, Lewis Y, CD24, folate receptor alpha, folate receptor beta, ERBBs, MUC1, EGFR, NCAM, PDGFR-beta, MAD-CT-2, Fos-related antigen, SSEA-4, neutrophil elastase, CAIX, HPV E6 E7, ML-IAP, NA17, ALK, androgen receptor plsialic acid, TRP-2, CYP1B1, PLAC1, GloboH, NY-BR-1, sperm protein 17, HMWMAA, beta human chorionic gonadotropin, AFP, thyroglobulin, RAGE-1, MN-CA IX, human telomerase reverse transcriptase, intestinal carboxyl esterase, and mut hsp 70-2; (iv) a solid tumor associated antigen present in/on a mesothelioma, a lung cancer, a pancreatic cancer, an esophageal adenocarcinoma, an ovarian cancer, a breast cancer, a colorectal cancer, a bladder cancer, or any combination thereof; (v) a tumor antigen that is associated with a hematological cancer; (vi) a tumor antigen present in a disease selected from acute leukemias (including, without limitation, B-cell acute lymphoid leukemia (“BALL”), T-cell acute lymphoid leukemia (“TALL”), acute lymphoid leukemia (ALL)), and chronic leukemias (including, without limitation, chronic myelogenous leukemia (CIVIL) and chronic lymphoid leukemia (CLL)), and (vii) a tumor antigen present in a therapy resistant cancer, including but not limited to myeloperoxidase and neutrophil elastase, which include any protein or peptide which is a non-material variant, mutation, fragment or derivative of ScFv, Fab, CDR Loops, CDR grafts, dAb and nanobody fragments, whether produced by a cell line, expression by a hybridoma, expression in vitro by bacteria, yeast or other host cell, or any other method of synthesis. In some embodiments, the DNase enzyme comprises an amino acid sequence having at least 90% sequence identity to SEQ ID NO: 4. In some embodiments, the deoxyribonuclease enzyme comprises an amino acid sequence having at least 90% sequence identity to SEQ ID NO: 53.

These and other aspects of the present invention will be apparent to those of ordinary skill in the art in the following description, claims and drawings.

### BRIEF DESCRIPTION OF THE DRAWINGS

FIG. 1 shows tumor volume (mm²) as measured by calipers and estimated using the ellipsoidal formula for control, CAR19 with DNasel, CAR19, and DNasel experimental groups. Intravenous (IV) injections of human recombinant DNase I enzyme provided substantial efficacy boost for CD19 CAR T cells.

FIG. 2 shows a survival graph for control, CAR19 with DNasel, CAR19, and DNaseI experimental groups demonstrating survival benefit from a combination of CAR T cells and AAV DNase I gene transfer.

FIGS. 3A-3C show flow cytometry analyses displaying the percentage of metastatic MC32a cells expressing PD-L1. Relative to control (FIG. 3A), PD-L1 expression was suppressed in metastatic MC32a cells from metastatic lesions from mice treated AAV-DNase I gene transfer (FIG. 3B), as well as with combination of CAR T cells (FIG. 3C).

FIG. 4 shows a graph of the percentage of Carcinoembryonic Antigen (CEA)-targeting CAR T cells in parenchyma of MC32a metastatic lesions. Data show an increased percentage of CAR T cells in metastatic lesions from mice treated with combination of CAR T cells and AAV DNase I gene transfer.

FIGS. 5A-5B show a schematic representation of construction of an exemplary viral vector. FIG. 5A shows a diagram of mDNasel mut P2A 4D5 IgG4 CAR comprising EF-1α promoter and CD3ζ domains (4,237 bp). FIG. 5B shows the full sequence map for a viral vector comprising mDNase I P2A IgG4 CAR (12,146 bp).

FIG. 6 shows a graph of DNase activity analyzed using a fluorescent probe in culture media of DNase I FL1-CAR-Ts, FL1-CAR-Ts, and untransduced control T cells. Activity was measured as pM/s (picometer per second). The data show gradual increase of deoxyribonuclease activity in culture media of DNasel FL1-CAR-Ts

FIG. 7 shows evaluation of cytotoxicity by Lactase Dehydrogenase (LDH) release assay for FL1-CAR-Ts, DNase I FL1-CAR-Ts and untransduced (mock) control T cells co-incubated with Raji-FL1 cells. Reprogramming of T cells to simultaneously express CAR and DNase I enzyme provided superior cytotoxicity against target lymphoma cells. E:T ratio, effector to target cell ratio.

FIG. 8 shows tumor volume (mm²) as measured by calipers and estimated using the ellipsoidal formula for placebo control (Group 1), FL1 CAR T cells (Group 2) and FL1 CAR T cells expressing hyperactive actin resistant mutant DNase I (SEQ ID NO: 5) (Group 3) experimental groups. Reprogramming of T cells to simultaneously express CAR and deoxyribonuclease enzyme provided superior efficacy for such dual reprogrammed T cells.

FIG. 9 shows the number of CAR-T cells in peripheral blood (determined by flow cytometry detection using biotinylated protein L and streptavidin conjugated with FITC). The data demonstrate that the combined use of DNase and CAR-T cell therapy significantly (for all p<0.05) increases the persistence of CAR-T cells in blood.

FIG. 10 demonstrates that the combined use of DNase and CAR-T cell therapy increases animal survival.

FIG. 11 shows percentage of animals with cytokine release syndrome (CRS) demonstrating that the use of DNase increases safety of CAR-T therapy and protects from CAR-T-related side effects.

### DETAILED DESCRIPTION OF THE INVENTION

The present invention relates to methods for treatment of cancers utilizing a combination of a deoxyribonuclease enzyme and adoptive cell immunotherapy comprising cells expressing a chimeric antigen receptor (CAR) or a T cell receptor (TCR). In particular embodiments, the deoxyribonuclease enzyme can be given parenterally or encoded by a vector or secreted by a cell comprising TCR or CAR.

### Definitions

Unless defined otherwise, all technical and scientific terms used herein have the same meaning as commonly understood by one of ordinary skill in the art to which this invention belongs.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

### DNase Enzymes

In certain aspects, the invention provides a method of treating a cancer in a subject in need thereof, comprising administering to the subject a deoxyribonuclease (DNase) enzyme and at least one cell comprising a chimeric antigen receptor (CAR) or a T cell receptor (TCR). In a related aspect, the invention provides a method of treating a cancer in a subject in need thereof, comprising administering to the subject a DNase enzyme and at least one cell comprising a chimeric antigen receptor (CAR) or a T cell receptor (TCR), wherein said DNase enzyme is effective to reduce expression of immunosuppressive proteins in tumor tissue and/or increase content of cells comprising a chimeric antigen receptor or T cell receptor within tumor tissue. In some embodiments, the DNase enzyme is administered parenterally as deoxyribonuclease enzyme protein. In some embodiments, the DNase enzyme is encoded by a vector. In some embodiments, the DNase enzyme is co-expressed by the cell comprising the chimeric antigen receptor (CAR) or the T cell receptor (TCR). In some embodiments, the DNase enzyme is used to increase the efficacy of CAR expressing cells in hypoxia environment.

Non-limiting examples of DNase enzymes that may be useful in any of the methods of the present invention include, e.g., DNase I, DNase X, DNase γ, DNase1L1, DNase1L2, DNase 1L3, DNase II, DNase IIα, DNase IIβ, Caspase-activated DNase (CAD), Endonuclease G (ENDOG), Granzyme B (GZMB), phosphodiesterase I, lactoferrin, acetylcholinesterase, and mutants or derivatives, variants or analogs thereof.

In some embodiments, the DNase may be a DNase I or a mutant or derivative thereof.

In some embodiments, the DNase I may be human DNase I or a mutant or derivative thereof. In some embodiments, the DNase I may be non-human DNase I or a mutant or derivative thereof, such as, but not limited to a rodent (e.g., a mouse) DNase I or a mutant or derivative thereof.

In some embodiments, the DNase enzyme comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to human DNase 1 enzyme. In some embodiments, the DNase enzyme comprises an amino acid sequence having at least 90%, sequence identity to human DNase I enzyme.

In some embodiments, the DNase enzyme comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to amino acids 21 to 305 of DNase 1-like 3 (D1L3) enzyme (SEQ ID NO: 51). In some embodiments, the DNase enzyme comprises an amino acid sequence having at least 90% sequence identity to amino acids 21 to 305 of DNase1-like 3 (D1L3) enzyme (SEQ ID NO: 51).

In some embodiments, the DNase I comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence of SEQ ID NO: 1. In some embodiments, the DNase I amino acid sequence is encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 22. In some embodiments, the DNase I comprises an amino acid sequence of SEQ ID NO: 1 as encoded by a nucleotide sequence of SEQ ID NO: 22.

In some embodiments, the DNase I comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence of SEQ ID NO: 4. In some embodiments, the DNase I amino acid sequence is encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 23. In some embodiments, the DNase I comprises an amino acid sequence of SEQ ID NO: 4 as encoded by a nucleotide sequence of SEQ ID NO: 23.

In some embodiments, the DNase I mutant may comprise one or more mutations in an actin binding site. In some embodiments, the one or more mutations in the actin-binding site are selected from a mutation at Gln-9, Glu-13, Thr-14, His-44, Asp-53, Tyr-65, Val-66, Val-67, Glu-69, Asn-74, Ala-114, and any combinations thereof. In some embodiments, one of the mutations in the actin-binding site is a mutation at Ala-114.

In some embodiments, the DNase I mutant comprises one or more mutations increasing DNase activity. In some embodiments, one or more mutations increasing DNase activity are selected from the group consisting of Q9R, E13R, E13K, T14R, T14K, H44R, H44K, N74K, A114F, and any combinations thereof. In some embodiments, one or more mutations increasing DNase activity are selected from the group consisting of Q9R, E13R, N74K, and A114F.

In some embodiments, the DNase I mutant comprises one or more mutations selected from the group consisting of H44C, H44N, L45C, V48C, G49C, L52C, D53C, D53R, D53K, D53Y, D53A, N56C, D58S, D58T, Y65A, Y65E, Y65R, Y65C, V66N, V67E, V67K, V67C, E69R, E69C, Al 14C, Al 14R, H44N:T46S, D53R:Y65A, D53R:E69R, H44A:D53R:Y65A, H44A:Y65A:E69R, H64N:V66S, H64N:V66T, Y65N:V67S, Y65N:V67T, V66N:S68T, V67N:E69S, V67N:E69T, S68N:P70S, S68N:P7OT, S94N:Y96S, S94N:Y96T, and any combinations thereof. In some embodiments, the DNase I mutant is a long acting form of DNase. In some embodiments, the DNase I mutant is a hyperactive variant form of DNase. In some embodiments, the DNase I mutant comprises the amino acid sequence SEQ ID NO: 5. In some embodiments, the DNase I mutant comprises the amino acid sequence SEQ ID NO: 2.

In some embodiments, the DNase I mutant comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence of SEQ ID NO: 5. In some embodiments, the DNase I mutant amino acid sequence is encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 21. In some embodiments, the DNase I mutant amino acid sequence is encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 18. In some embodiments, the DNasel mutant comprises the mutations Q9R, E13R N74K and A114F. In some embodiments, the DNase I mutant comprises the sequence of SEQ ID NO: 5. In some embodiments, the DNase I mutant comprises an amino acid sequence of SEQ ID NO: 5 as encoded by a nucleotide sequence of SEQ ID NO: 21.

In some embodiments, the DNase I mutant comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence of SEQ ID NO: 2. In some embodiments, the DNase I mutant amino acid sequence is encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 19. In some embodiments, the DNasel mutant comprises the mutations Q9R, E13R N74K and A114F. In some embodiments, the DNase I mutant comprises the sequence of SEQ ID NO: 2. In some embodiments, the DNase I mutant comprises an amino acid sequence of SEQ ID NO: 2 as encoded by a nucleotide sequence of SEQ ID NO: 19.

In some embodiments, the DNase I mutant consists of the sequence of SEQ ID NO: 2 or SEQ ID NO: 5.

In some embodiments of any of the methods of the invention, the sequence encoding the DNase comprises a secretory signal sequence, wherein said secretory signal sequence mediates effective secretion of the enzyme into the hepatic porto-sinusoidal circulation upon administration of the vector to the subject. In some embodiments, the secretory signal sequence is selected from the group consisting of DNase I secretory signal sequence, IL2 secretory signal sequence, albumin secretory signal sequence, β-glucuronidase secretory signal sequence, alkaline protease secretory signal sequence, and fibronectin secretory signal sequence. In some embodiments, the secretory signal sequence comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence MRGMKLLGALLALAALLQGAVS (SEQ ID NO: 6). In some embodiments, the secretory signal sequence comprises the sequence MRGMKLLGALLALAALLQGAVS (SEQ ID NO: 6). In some embodiments, the secretory signal sequence consists of the sequence MRGMKLLGALLALAALLQGAVS (SEQ ID NO: 6). In some embodiments, the secretory signal sequence comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence MYRMQLLSCIALSLALVTNS (SEQ ID NO: 7). In some embodiments, the secretory signal sequence comprises the sequence MYRMQLLSCIALSLALVTNS (SEQ ID NO: 7). In some embodiments, the secretory signal sequence consists of the sequence MYRMQLLSCIALSLALVTNS (SEQ ID NO: 7).

In some embodiments, the DNase enzyme is selected from the group consisting of DNase I, DNase X, DNase γ, DNase1L1, DNase1L2, DNase 1L3, DNase II, DNase IIα, DNase IIβ, Caspase-activated DNase (CAD), Endonuclease G (ENDOG), Granzyme B (GZMB), phosphodiesterase I, lactoferrin, acetylcholinesterase, and mutants or derivatives, variants or analogs thereof. In some embodiments, the DNase enzyme is DNase I or a mutant or derivative thereof. In some embodiments, the DNase I is a human DNase I or a mutant or derivative thereof. In some embodiments, the DNase I mutant comprises one or more mutations in an actin binding site. In some embodiments, the one or more mutations in the actin-binding site are selected from a mutation at Gln-9, Glu-13, Thr-14, His-44, Asp-53, Tyr-65, Val-66, Val-67, Glu-69, Asn-74, Ala-114, and any combinations thereof. In some embodiments, one of the mutations in the actin-binding site is a mutation at Ala-114. In some embodiments, the DNase I mutant comprises one or more mutations increasing DNase activity. In some embodiments, one or more mutations increasing DNase activity are selected from the group consisting of Q9R, E13R, E13K, T14R, T14K, H44R, H44K, N74K, A114F, and any combinations thereof. In some embodiments, one or more mutations increasing DNase activity are selected from the group consisting of Q9R, E13R, N74K and A114F, and any combinations thereof. In some embodiments, the DNase I mutant comprises a sequence having at least 80% or at least 85% or at least 90% or at least 95% sequence identity to the sequence of SEQ ID NO: 5. In some embodiments, the DNase I mutant comprises the mutations Q9R, E13R, N74K, and A114F. In some embodiments, the DNase I mutant comprises the sequence of SEQ ID NO: 5. In some embodiments, the DNAse I mutant consists of the sequence of SEQ ID NO: 5. In some embodiments, the DNase I mutant comprises a sequence having at least 80% or at least 85% or at least 90% or at least 95% sequence identity to the sequence of SEQ ID NO: 2. In some embodiments, the DNase I mutant comprises the mutations Q9R, E13R, N74K, and A114F. In some embodiments, the DNase I mutant comprises the sequence of SEQ ID NO: 2. In some embodiments, the DNAse I mutant consists of the sequence of SEQ ID NO: 2. In some embodiments, the DNase I mutant comprises one or more mutations selected from the group consisting of H44C, H44N, L45C, V48C, G49C, L52C, D53C, D53R, D53K, D53Y, D53A, N56C, D58S, D58T, Y65A, Y65E, Y65R, Y65C, V66N, V67E, V67K, V67C, E69R, E69C, Al 14C, Al 14R, H44N:T46S, D53R:Y65A, D53R:E69R, H44A:D53R:Y65A, H44A:Y65A:E69R, H64N:V66S, H64N:V66T, Y65N:V67S, Y65N:V67T, V66N:S68T, V67N:E69S, V67N:E69T, S68N:P7OS, S68N:P70T, S94N:Y96S, S94N:Y96T, and any combinations thereof. In some embodiments, the nucleic acid encodes a DNase I comprising the sequence SEQ ID NO: 4. In some embodiments, the nucleic acid comprises a nucleotide sequence which is at least 85% or at least 90% or at least 95% identical to SEQ ID NO: 23. In some embodiments, the nucleic acid comprises the nucleotide sequence SEQ ID NO: 23. In some embodiments, the nucleic acid encodes a DNase I comprising the sequence SEQ ID NO: 1. In some embodiments, the nucleic acid comprises a nucleotide sequence which is at least 85% or at least 90% or at least 95% identical to SEQ ID NO: 22. In some embodiments, the nucleic acid comprises the nucleotide sequence SEQ ID NO: 22. In some embodiments, the nucleic acid comprises a nucleotide sequence which is at least 85% or at least 90% or at least 95% identical to SEQ ID NO: 32. In some embodiments, the nucleic acid comprises the nucleotide sequence SEQ ID NO: 32. In some embodiments, the nucleic acid encodes a DNase I comprising the sequence SEQ ID NO: 24. In some embodiments, the nucleic acid comprises a nucleotide sequence which is at least 85% or at least 90% or at least 95% identical to SEQ ID NO: 29. In some embodiments, the nucleic acid comprises the nucleotide sequence SEQ ID NO: 29. In some embodiments, the nucleic acid encodes a DNase I comprising the sequence of SEQ ID NO: 26. In some embodiments, the nucleic acid comprises a nucleotide sequence which is at least 85% or at least 90% or at least 95% identical to SEQ ID NO: 28. In some embodiments, the nucleic acid comprises the nucleotide sequence SEQ ID NO: 28. In some embodiments, the nucleic acid encodes a DNase I mutant comprising the sequence SEQ ID NO: 5. In some embodiments, the nucleic acid comprises a nucleotide sequence which is at least 85% or at least 90% or at least 95% identical to SEQ ID NO: 21. In some embodiments, the nucleic acid comprises the nucleotide sequence SEQ ID NO: 21. In some embodiments, the nucleic acid encodes a DNase I mutant comprising the sequence SEQ ID NO: 2. In some embodiments, the nucleic acid comprises a nucleotide sequence which is at least 85% or at least 90% or at least 95% identical to SEQ ID NO: 19. In some embodiments, the nucleic acid comprises the nucleotide sequence SEQ ID NO: 19. In some embodiments, the DNase enzyme is a fusion protein comprising (i) a DNase enzyme or a fragment thereof linked to (ii) an albumin or an Fc or a fragment thereof. In some embodiments, the sequence encoding the DNase enzyme comprises a sequence encoding a secretory signal sequence, wherein said secretory signal sequence mediates effective secretion of the enzyme. In some embodiments, the secretory signal sequence is selected from the group consisting of DNase I secretory signal sequence, IL2 secretory signal sequence, the albumin secretory signal sequence, the β-glucuronidase secretory signal sequence, the alkaline protease secretory signal sequence, and the fibronectin secretory signal sequence. In some embodiments, the secretory signal sequence comprises the sequence MRGMKLLGALLALAALLQGAVS (SEQ ID NO: 6) or MYRMQLLSCIALSLALVTNS (SEQ ID NO: 7). In some embodiments, the secretory signal sequence consists of the sequence MRGMKLLGALLALAALLQGAVS (SEQ ID NO: 6) or MYRMQLLSCIALSLALVTNS (SEQ ID NO: 7). In some embodiments, the secretory signal sequence comprises a sequence having at least 80% or at least 85% or at least 90% or at least 95% sequence identity to the sequence of MRGMKLLGALLALAALLQGAVS (SEQ ID NO: 6) or a sequence having at least 85% or at least 90% or at least 95% sequence identity to the sequence of MYRMQLLSCIALSLALVTNS (SEQ ID NO: 7). In some embodiments, the sequence encoding the secretory signal sequence comprises a nucleotide sequence which is at least 85% or at least 90% or at least 95% identical to SEQ ID NO: 20. In some embodiments, the sequence encoding the secretory signal sequence comprises the nucleotide sequence of SEQ ID NO: 20. In some embodiments, the secretory signal sequence comprises the sequence MRYTGLMGTLLTLVNLLQLAGT (SEQ ID NO: 25). In some embodiments, the secretory signal sequence consists of the sequence MRYTGLMGTLLTLVNLLQLAGT (SEQ ID NO: 25). In some embodiments, the secretory signal sequence comprises a sequence having at least 80% or at least 85% or at least 90% or at least 95% sequence identity to the sequence of MRYTGLMGTLLTLVNLLQLAGT (SEQ ID NO: 25). In some embodiments, the sequence encoding the secretory signal sequence comprises the nucleotide sequence SEQ ID NO: 27. In a separate embodiment, the nucleic acid of the invention comprises the sequence of SEQ ID NO: 30 or SEQ ID NO: 31.

### AAV Vectors

In some embodiments, the DNase enzyme disclosed herein may be encoded by a vector (e.g., a gene therapy vector). In some embodiments, the vector is a recombinant adeno-associated virus (rAAV) expression vector comprising (i) a capsid protein and (ii) a nucleic acid comprising a promoter operably linked to a nucleotide sequence encoding a deoxyribonuclease (DNase) enzyme.

In some embodiments, the vector is a viral vector. Non-limiting examples of viral vectors include, e.g., adeno-associated virus (AAV) vectors, adenoviral vectors, retroviral vectors (e.g., lentivirus vectors), and hepatotropic viral vectors (e.g., hepatitis B virus (HBV) vectors).

In certain embodiments, the vector of the present invention is a parvovirus vector, such as an adeno-associated viral (AAV) vector. The term “parvovirus” as used herein encompasses the family Parvoviridae, including autonomously-replicating parvoviruses and dependoviruses. The autonomous parvoviruses include members of the genera Parvovirus, Erythrovirus, Densovirus, Iteravirus, and Contravirus. Autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, B 19 virus, and any other autonomous parvovirus now known or later discovered. Other autonomous parvoviruses are known to those skilled in the art. See, e.g., Bernard N. Fields et al., Virology, Vol. 2, Chapter 69 (4th ed., Lippincott-Raven Publishers).

In some embodiments, the parvovirus vector is a single-stranded parvovirus vector, such as an AAV vector. AAV (genus Dependovirus) normally infects humans (e.g., serotypes 1, 2, 3A, 3B, 4, 5, and 6) or primates (e.g., serotypes 1 and 4), or other warm-blooded animals (e.g., bovine, canine, equine, and ovine AAVs). Further information on parvoviruses and other members of the Parvoviridae is provided, e.g., in Kenneth I. Berns, “Parvoviridae: The Viruses and Their Replication,” Chapter 69 in Fields Virology (3d Ed. 1996).

AAV vectors disclosed herein may be derived from any AAV serotype, including combinations of serotypes (e.g., “pseudotyped” AAV) or from various genomes (e.g., single-stranded or self-complementary). A “serotype” is traditionally defined on the basis of a lack of cross-reactivity between antibodies to one virus as compared to another virus. Such cross-reactivity differences are usually due to differences in capsid protein sequences/antigenic determinants (e.g., due to VP1, VP2, and/or VP3 sequence differences). Non-limiting examples of AAV serotypes which can be used to develop the AAV expression vectors of the invention include, e.g., AAV serotype 1 (AAV1), AAV2, AAV3 (including types 3A and 3B), AAV4, AAVS, AAV6, AAV7, AAV8, AAV9, AAV10, AAV11, AAV12, AAVrh10 (as disclosed, e.g., in U.S. Pat. No. 9,790,472, Int. Pat. Appl. Pub. No. WO2017180857 and WO2017/180861), AAV-LK03, AAV-LK06, AAV-LK12 (as disclosed, e.g., in Wang et al., Mol. Ther., 2015, 23(12):1877-1887), AAV-KP1(as disclosed, e.g., in Int. Pat. Appl. Pub. No. WO2019191701A1), AAVhu37 (as disclosed, e.g., in Int. Pat. Appl. Pub. No. WO2017180857), AAVrh64R1 (as disclosed, e.g., in Int. Pat. Appl. Pub. No. WO2017180857), Anc80 (based on a predicted ancestor of serotypes AAV1, AAV2, AAV8 and AAV9; see Zinn et al., Cell Rep., 2015, 12(67): 1056-1068), avian AAV, bovine AAV, canine AAV, equine AAV, ovine AAV, and any chimeras thereof. See, e.g., Fields et al., Virology, volume 2, chapter 69 (4th ed., Lippincott-Raven Publishers).

A number of putative new AAV serotypes and clades have been identified (see, e.g., Gao et al., (2004) J. Virology 78:6381-6388; Moris et al., (2004) Virology 33-:375-383). The genomic sequences of the various serotypes of AAV and the autonomous parvoviruses, as well as the sequences of the terminal repeats, Rep proteins, and capsid subunits are known in the art, by way of example, Srivistava et al., (1983) J. Virology 45:555; Chiorini et al., (1998) J. Virology 71:6823; Chiorini et al, (1999) J. Virology 73:1309; Bantel-Schaal et al., (1999) J. Virology 73:939; Xiao et al., (1999) J. Virology 73:3994; Muramatsu et al., (1996) Virology 221: 208; Shade et al., (1986) J. Virol. 58:921; Gao et al., (2002) Proc. Nat. Acad. Sci. USA 99:11854; Moris et al., (2004) Virology 33-: 375-383; GenBank Accession number U89790; GenBank Accession number J01901; GenBank Accession number AF043303; GenBank Accession number AF085716; GenBank Accession number NC 006152; GenBank Accession number Y18065; GenBank Accession number NC 006260; GenBank Accession number NC_006261; International Patent Publication Nos. WO 00/28061, WO 99/61601, WO 98/11244; and U.S. Pat. No. 6,156,303, the disclosures of which are incorporated by reference herein for teaching parvovirus and AAV nucleic acid and amino acid sequences.

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP1, VP2 and VP3) form the capsid. The terminal 145 nucleotides are self-complementary and are organized so that an energetically stable intramolecular duplex forming a T-shaped hairpin may be formed. These hairpin structures function as an origin for viral DNA replication, serving as primers for the cellular DNA polymerase complex. Following wtAAV infection in mammalian cells the Rep genes (i.e. Rep78 and Rep52) are expressed from the P5 promoter and the P19 promoter, respectively and both Rep proteins have a function in the replication of the viral genome.

In some embodiments, recombinant AAV (rAAV) vectors comprise one or more nucleotide sequences of interest that are flanked by at least one parvoviral or AAV inverted terminal repeat sequence (ITR). Such rAAV vectors can be replicated and packaged into viral particles when produced in a packaging cell that is expressing AAV rep and cap gene products (i.e., AAV Rep and Cap proteins). The terms “AAV Cap protein” or “AAV capsid protein”, as used herein, refer to a polypeptide having at least one functional activity of a native AAV Cap protein (e.g., VP1, VP2, VP3). Examples of functional activities of Cap proteins (e.g., VP1, VP2, VP3) include the ability to induce formation of a capsid, facilitate accumulation of single-stranded DNA, facilitate AAV DNA packaging into capsids (i.e., encapsidation), bind to cellular receptors, and facilitate entry of the virion into host cells.

In some embodiments, the AAV vectors may comprise desired proteins or protein variants. A “variant” as used herein, refers to an amino acid sequence that is altered by one or more amino acids. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Analogous minor variations may also include amino acid deletions or insertions, or both.

AAV vectors are packaged into AAV viral capsids. The sequence of an AAV viral capsid protein defines numerous features of a particular AAV vector. For example, the capsid protein affects capsid structure and assembly, interactions with AAV nonstructural proteins such as Rep and AAP proteins, interactions with host body fluids and extracellular matrix, clearance of the virus from the blood, vascular permeability, antigenicity, reactivity to neutralizing antibodies, tissue/organ/cell type tropism, efficiency of cell attachment and internalization, intracellular trafficking routes, virion uncoating rates, among others. The sequence of a capsid protein (e.g., VP3) may be altered to enhance delivery to the liver.

AAV constructs may comprise a sequence encoding one or more capsid proteins (VP1 and/or VP2, and/or VP3 capsid proteins, preferably just VP3 capsid protein) which package the polynucleotide sequence disclosed herein. The sequences coding for the capsid protein(s) for use in the context of the present invention may be taken from any of the known 42 serotypes, such as, e.g., AAV1, AAV2, AAV3, AAV4, AAV5, AAV6, AAV7, AAV8, AAV9, Anc80, or newly developed AAV-like particles obtained by, e.g., capsid shuffling techniques and/or use of AAV capsid libraries. For some non-limiting examples of capsid protein sequences, see, e.g., U.S. Pat. Nos. 9,790,472; 9,677,089; 7,282,199; Int. Pat. Appl. Publ. Nos. WO 2015/054653, WO2017/180857 (AAV8, AAV9, AAVrh10, AAVhu37, AAVrh64R1), WO2017/180861 (AAVrh10), WO2017/180854 (AAV8 mutants), and Wang et al., Mol. Ther., 2015, 23(12):1877-1887 (AAV8, AAVrh10, AAV3B, and AAV-LK03). When the sequences encoding the capsid proteins derive from a different AAV serotype as the ITRs, the AAV construct is known as a “hybrid” parvovirus genome (i.e., in which the AAV capsid and the AAV terminal repeat(s) are from different AAV) as described in Int. Pat. Appl. Publ. No. WO 00/28004 and Chao et al., (2000) Molecular Therapy 2:619.

In some embodiments, the capsid protein(s) mediates efficient targeting of the AAV vector to the liver. In some embodiments, the capsid protein(s) mediate preferential targeting of the AAV vector to the liver. Some capsid proteins (e.g., VP3 of Anc80, AAV8 and AAV3B) are naturally liver-specific. The invention also encompasses the use of AAV capsid mutants which enhance liver targeting and/or liver specificity. Non-limiting examples of such point mutations to the AAV8 capsid sequence include, e.g., S279A, S671A, K137R, and T252A, as well as AAV8 capsid mutations disclosed in Int. Pat. Appl. Pub. No. WO2017/180854 (e.g., AAV3G1, AAVT20 or AAVTR1, VP3 mutations in amino acids 263-267 [e.g., 263NGTSG267->SGTH or 263NGTSG267->SDTH (“NGTSG” is disclosed as SEQ ID NO: 54; “SGTH” is disclosed as SEQ ID NO: 55, and “SDTH” is disclosed as SEQ ID NO: 56)] and/or amino acids 457-459 [e.g., 457TAN459->SRP], and/or amino acids 455-459 [e.g., 455GGTAN459 ->DGSGL (“GGTAN” is disclosed as SEQ ID NO: 57 and “DGSGL” is disclosed as SEQ ID NO: 58) and/or amino acids 583-597).

In some embodiments, the capsid protein(s) mediates efficient targeting of the AAV vector to the nervous system. In some embodiments, the capsid protein(s) mediate preferential targeting of the AAV vector both to the liver and to the nervous system. Some capsid proteins would naturally target both the liver and the nervous system. The invention also encompasses the use of AAV capsid mutants which enhance targeting to the liver and/or the nervous system.

In some embodiments, the capsid protein comprises one or more mutations which improve efficiency and/or specificity of the delivery of the vector to the liver and/or nervous system as compared to the corresponding wild-type capsid protein. In one embodiment, the improved efficiency and/or specificity of the delivery of the vector to the liver results in a substantially increased expression of the enzyme in the liver as compared to other tissues and organs. In one embodiment, the improved efficiency and/or specificity of the delivery of the vector to the nervous system results in a substantially increased expression of the enzyme in the nervous system as compared to other tissues and organs. In one embodiment, the capsid protein comprises VP3. In one embodiment, the one or more mutations in the capsid protein are selected from the group consisting of S279A, S671A, K137R, T252A, and any combinations thereof. In one embodiment, the one or more mutations in the capsid protein include mutation K137R. In one embodiment, the capsid protein comprises the sequence SEQ ID NO: 34. In one embodiment, the capsid protein consists of the sequence SEQ ID NO: 34

In some embodiments, the capsid protein comprises one or more mutations selected from the group consisting of S279A, S671A, K137R, T252A, and any combinations thereof. In one embodiment, the one or more mutations in the capsid protein include mutation K137R. In one embodiment, the capsid protein comprises the sequence SEQ ID NO:3 [Anc80]. In one embodiment, the capsid protein consists of the sequence SEQ ID NO:3 [Anc80]. In one embodiment, the capsid protein comprises the sequence SEQ ID NO:9 [Anc80]. In one embodiment, the capsid protein consists of the sequence SEQ ID NO:9 [Anc80]. In one embodiment, the capsid protein comprises the sequence SEQ ID NO:34 [Anc80L65]. In one embodiment, the capsid protein consists of the sequence SEQ ID NO:34 [Anc80L65]. In one embodiment, the capsid protein comprises the sequence SEQ ID NO:35 [Anc80L65 variant]. In one embodiment, the capsid protein consists of the sequence SEQ ID NO:35 [Anc80L65 variant]. In one embodiment, the capsid protein is a mutant AAV8 capsid protein such as, e.g., AAV3G1, AAVT20 or AAVTR1, or another mutant capsid protein disclosed in Int. Pat. Appl. Pub. No. WO2017/180854 (e.g., comprising VP3 mutations in amino acids 263-267 [e.g., 263NGTSG267->SGTH or 263NGTSG267->SDTH (“NGTSG” is disclosed as SEQ ID NO: 54; “SGTH” is disclosed as SEQ ID NO: 55, and “SDTH” is disclosed as SEQ ID NO: 56)] and/or amino acids 457-459 [e.g., 457TAN459->SRP], and/or amino acids 455-459 [e.g., 455GGTAN459->DGSGL] (“GGTAN” is disclosed as SEQ ID NO: 57 and “DGSGL” is disclosed as SEQ ID NO: 58) and/or amino acids 583-597). In one embodiment, the capsid protein comprises the sequence SEQ ID NO: 47 [AAV-LK03]. In one embodiment, the capsid protein consists of the sequence SEQ ID NO: 47 [AAV-LK03]. In one embodiment, the capsid protein comprises the sequence SEQ ID NO: 49 [AAV-KP1]. In one embodiment, the capsid protein consists of the sequence SEQ ID NO: 49 [AAV-KP1].

The AAV vectors disclosed herein include a nucleic acid encoding a deoxyribonuclease (DNase) enzyme. In various embodiments, the nucleic acid also may include one or more regulatory sequences allowing expression and secretion of the encoded enzyme, such as e.g., a promoter, enhancer, polyadenylation signal, an internal ribosome entry site (IRES), a sequence encoding a protein transduction domain (PTD), a secretory signal sequence, and the like. Thus, in some embodiments, the nucleic acid may comprise a promoter region operably linked to the coding sequence to cause or improve expression of the protein of interest in transfected cells. Such a promoter may be ubiquitous, cell- or tissue-specific, strong, weak, regulated, chimeric, etc., for example to allow efficient and stable production of the protein in the liver and/or nervous system. The promoter may be homologous to the encoded protein, or heterologous, although generally promoters of use in the disclosed methods are functional in human cells. Examples of regulated promoters include, without limitation, Tet on/off element-containing promoters, rapamycin-inducible promoters, tamoxifen-inducible promoters, and metallothionein promoters. Other promoters that may be used include promoters that are tissue specific, e.g., for liver, nervous system and/or intestine. Non-limiting examples of liver-specific promoters include, e.g., the albumin promoter (Alb), human alpha-1 anti-trypsin (hAAT) promoter, thyroxine binding globulin (TBG), apolipoprotein E hepatic control region promoter, apolipoprotein A-II (APOA2) promoter, serpin peptidase inhibitor, Glade A, member 1 (SERPINA1) (hAAT) promoter, cytochrome P450 family 3, subfamily A polypeptide 4 (CYP3A4) promoter, microRNA 122 (miR-122) promoter, liver-specific IGF-II promoter P1, murine transthyretin (MTTR) promoter, and the alpha-fetoprotein (AFP) promoter. Non-limiting examples of nervous system-specific promoters include, e.g., microglia-specific promoters (e.g., F4/80, CD68, TMEM119, CX3CR1, CMV, Iba1), myeloid-specific promoters (e.g., TTR, CD11b, c-fes), neuron specific promoters (e.g., CMV, NSE, synapsin [SynI, SynII], CamKII, α-CaMKII, VGLUT1), and other neural and glial cell (e.g., oligodendrocytes astrocytes) type-specific promoters (e.g., GFAP). Non-limiting examples of intestine-specific promoters include, MUC2, Villin, T3b, CB/CMV, GFAP, miCMV, CMV+I, tetO-CMV, and β-acti-CMV. Non-limiting examples of ubiquitous promoters include, e.g., viral promoters such as the CMV promoter, the RSV promoter, the SV40 promoter, etc., and cellular promoters such as the phosphoglycerate kinase (PGK) promoter, EF 1 a promoter, CMVE/CAG promoter system, and the β-actin promoter. In some embodiments, the promoter is specific for tumor originator tissue or metastasis target tissue.

In some embodiments, any of the AAV vectors disclosed herein may comprise one or more enhancers located upstream or downstream of the promoter. In some embodiments, the one or more enhancers may be selected from the group consisting of an apolipoprotein E (ApoE) enhancer, an alpha fetoprotein enhancer, a TTR enhancer, an LSP enhancer, an α1-microglobulin/bikunin enhancer, an albumin gene enhancer (Ealb), a nPE2 enhancer, a Gal4 enhancer, a foxP2 enhancer, aMef2 enhancer, a CMV enhancer, and any combination thereof. In one embodiment, the enhancer is an apolipoprotein E (ApoE) enhancer. In some embodiments, the enhancer is a hepatic control region (HCR) enhancer. In some embodiments, the enhancer comprises a sequence having at least 80% or at least 85% or at least 90% or at least 95% sequence identity to SEQ ID NO: 17. In one embodiment, the enhancer comprises the sequence SEQ ID NO: 17. In one embodiment, the enhancer consists of the sequence SEQ ID NO: 17.

In one embodiment of any of the vectors described above, the nucleic acid further comprises a polyadenylation signal operably linked to the nucleotide sequence encoding the DNase enzyme.

In some embodiments, the nucleic acid further comprises a Kozak sequence. In one specific embodiment, the Kozak sequence comprises the sequence of 5′-GCCGCCACC-3′ (SEQ ID NO: 33). In one embodiment, the nucleic acid comprises a nucleotide sequence which is at least 80% identical to SEQ ID NO: 30. In one embodiment, the nucleic acid comprises a nucleotide sequence which is at least 85% identical to SEQ ID NO: 30. In one embodiment, the nucleic acid comprises a nucleotide sequence which is at least 90% identical to SEQ ID NO: 30. In one embodiment, the nucleic acid comprises a nucleotide sequence which is at least 95% identical to SEQ ID NO: 30. In one embodiment, the nucleic acid comprises the nucleotide sequence SEQ ID NO: 30.

In one embodiment of any of the vectors described above, the nucleic acid further comprises a post-transcriptional regulatory element. In one embodiment, the post-transcriptional regulatory element is a woodchuck hepatitis post-transcriptional regulatory element (WPRE). In one embodiment, the WPRE does not encode a functional X protein. In one embodiment, the post-transcriptional regulatory element comprises a sequence having at least 80% or at least 85% or at least 90% or at least 95% sequence identity to SEQ ID NO: 16. In one embodiment, the post-transcriptional regulatory element comprises the sequence SEQ ID NO: 16. In one embodiment, the post-transcriptional regulatory element consists of the sequence SEQ ID NO: 16.

The AAV vectors of the invention may also contain non-resolvable terminal repeats. The expression “non-resolvable terminal repeat”, as used herein, relates to terminal repeats which are not recognized by and resolved (i.e., “nicked”) by the AAV Rep proteins, such that resolution of the terminal repeat is substantially reduced (e.g., by at least about 50%, 60%, 70%, 80%. 90%, 95%, 98% or greater as compared with a resolvable terminal repeat) or eliminated. Such non-resolvable terminal repeats may be naturally-occurring terminal repeat sequences (including altered forms thereof) and, for example, can be derived from a parvovirus, including an AAV, or can be from another virus or, as a further alternative, can be partially or completely synthetic. The non-resolvable terminal repeat may be a non-AAV viral sequence that is not recognized by the AAV Rep proteins, or it can be an AAV terminal repeat that has been modified (e.g., by insertion, substitution and/or deletion) so that it is no longer recognized by the AAV Rep proteins. Further, a non-resolvable terminal repeat can be any terminal repeat that is non-resolvable under the conditions used to produce the virus vector. Further, an AAV terminal repeat can be modified so that resolution by the AAV Rep proteins is substantially reduced or eliminated. The non-resolvable terminal repeat can be any inverted repeat sequence that forms a hairpin structure and cannot be nicked by the AAV Rep proteins.

The inverted terminal repeats (ITR) are typically present in at least two copies in the AAV vector, typically flanking the expression cassette containing the nucleotide sequence(s) encoding a DNase enzyme. The ITRs typically will be at the 5′ and 3′ ends of the nucleotide sequence(s) encoding a DNase enzyme but need not be contiguous thereto. The ITRs can be the same or different from each other. The term “terminal repeat” includes any viral terminal repeat and/or partially or completely synthetic sequences that form hairpin structures and function as an inverted terminal repeat, such as the “double-D sequence” as described in U.S. Pat. No. 5,478,745 to Samulski et al. An “AAV terminal repeat” may be from any AAV, including but not limited to serotypes 1, 2, 3, 4, 5, 6, 7, 8, 9, 10, 11, 12, Anc80, or any other AAV now known or later discovered. The AAV terminal repeat need not have a wild-type sequence (e.g., a wild-type sequence may be altered by insertion, deletion, truncation or missense mutations), as long as the terminal repeat mediates the desired functions, e.g., replication, nicking, virus packaging, integration, and/or provirus rescue, and the like. The vector construct can comprise one or more (e.g., two) AAV terminal repeats, which may be the same or different. Further, the one or more AAV terminal repeats can be from the same AAV serotype as the AAV capsid, or can be different. In particular embodiments, the vector construct comprises an AAV1, AAV2, AAV3, AAV4, AAVS, AAV6, AAV7, AAV8, AAV9, AAV10, AAV11, AAV12, and/or Anc80 terminal repeat.

Parvoviral ITR nucleotide sequences are typically palindromic sequences, comprising mostly complementary, symmetrically arranged sequences also referred to as “A,” “B,” and “C” regions. The ITR functions as an origin of replication, a site having a “cis” role in replication, i.e., being a recognition site for trans acting replication proteins such as e.g. Rep 78 (or Rep68) which recognize the palindrome and specific sequences internal to the palindrome. One exception to the symmetry of the ITR sequence is the “D” region of the ITR. It is unique (not having a complement within one ITR). Nicking of single-stranded DNA occurs at the junction between the A and D regions. It is the region where new DNA synthesis initiates. The D region normally sits to one side of the palindrome and provides directionality to the nucleic acid replication step. A parvovirus replicating in a mammalian cell typically has two ITR sequences. It is, however, possible to engineer an ITR so that binding sites are on both strands of the A regions and D regions are located symmetrically, one on each side of the palindrome. On a double-stranded circular DNA template (e.g., a plasmid), the Rep78- or Rep68-assisted nucleic acid replication then proceeds in both directions and a single ITR suffices for parvoviral replication of a circular vector. Thus, one ITR nucleotide sequence can be used in the context of the present invention. Two or another even number of regular ITRs can be used.

Functional ITR sequences are necessary for the replication, rescue and packaging of AAV virions. The ITR sequences may be wild type sequences or may have at least 80%, 85%, 90%, 95%, or 100% sequence identity with wild type sequences. The ITR sequences may be altered by for example in insertion, mutation, deletion or substitution of nucleotides, as long as they remain functional. In this context, functionality refers to the ability to direct packaging of the genome into the capsid shell and then allow for expression in the host cell to be transduced or target cell.

The AAV vector can comprise single stranded or double stranded (self-complementary) DNA. The single stranded nucleic acid molecule is either sense or antisense strand, as both polarities are equally capable of gene expression. The AAV vector may further comprise a marker or reporter gene, such as a gene for example encoding an antibiotic resistance gene, a fluorescent protein (e.g., GFP) or a gene encoding a chemically, enzymatically or otherwise detectable and/or selectable product (e.g., lacZ, aph, etc.) known in the art.

AAV expression vectors may comprise a nucleic acid that may include a secretory signal sequence allowing secretion of the encoded DNase enzyme from the transduced cell. Non-limiting examples of such secretory signal sequences include, e.g., DNase I secretory signal sequence, IL2 secretory signal sequence, albumin secretory signal sequence, β-glucuronidase secretory signal sequence, alkaline protease secretory signal sequence, and fibronectin secretory signal sequence.

In some embodiments, an AAV8-based or an Anc80-based vector is used as the expression vector. The AAV8 and Anc80 vectors are particularly suited for liver targeting and expression. For example, both AAV8 and Anc80 vectors can transfect liver cells with greater efficiency as compared to an AAV2 vector. Both AAV8 and Anc80 also induce lower amounts of neutralizing antibodies than some of the other AAV vectors.

An AAV8 vector or an Anc80 vector comprising a nucleotide encoding a DNase enzyme can be administered intrahepatically (e.g., via direct organ injection) or systemically, e.g., by intravenous injection, with the AAV8 or Anc80 vector effective to transfect liver cells and mediate effective production of the encoded DNase enzyme and its secretion in the periendothelial space and the space of Disse. In some embodiments, an Anc80 capsid protein (e.g., Anc80 VP1 capsid protein comprising the sequence SEQ ID NO: 3 or SEQ ID NO: 9, or an Anc80L65 VP1 capsid protein comprising the sequence of SEQ ID NO:34, or a variant Anc80L65 VP1 capsid protein comprising the sequence of SEQ ID NO: 35) is encoded by a nucleotide sequence in the expression vector. In some embodiments, an AAV8 capsid protein (e.g., AAV8 VP1 or AAV8 VP3) is encoded by a nucleotide sequence in the expression vector. Peripheral administration of the AAV vectors of the invention may include systemic injections, such as, e.g., intramuscular, intravenous, intraperitoneal, and intra-arterial injections.

In some embodiments, an AAV3-based, or an AAV-LK03, or an AAV-KP1 vector is used as the expression vector. The AAV3, AAV-LKO3 and AAV-KP1 vectors are particularly suited for liver targeting and expression. For example, AAV3, AAV-LK03 and AAV-KP1 vectors can transfect liver cells with greater efficiency as compared to an AAV2 vector. Both AAV-LK03 and AAV-KP1 also induce lower amounts of neutralizing antibodies than some of the other AAV vectors.

The desired doses of the DNase enzyme encoding AAV vectors of the invention may be easily adapted by the skilled artisan, e.g., depending on the disease condition, the subject, the treatment schedule, etc. In some embodiments, from 10e5 to 101e4 recombinant viral particles are administered per dose, for example, from 10e6 to 10e11, from 10e7 to 10e11, or from 108 to 1014. In other embodiments, exemplary doses for achieving therapeutic effects may include titers of at least about 10e5, 10e6, 10e7, 10e8, 10e9, 10e10 or 10e11 recombinant viral particles or more.

The exogenous targeting sequence(s) may replace or substitute part or all of a major capsid subunit (e.g., VP3). As a further alternative, more than one exogenous targeting sequence, e.g., two, three, four, five or more sequences, may be introduced into the virion capsid. In alternative embodiments, insertions and substitutions within the minor capsid subunits (e.g., VP1 and VP2) may be undertaken. For AAV capsids, insertions or substitutions in VP2 or VP3 may be undertaken.

The native virion tropism may be reduced or abolished by insertion or substitution of the amino acid sequence. Alternatively, the insertion or substitution of the exogenous amino acid sequence may target the virion to a particular cell type(s). The exogenous targeting sequence may be any amino acid sequence encoding a protein or peptide that alters the tropism of the virion. In particular embodiments, the targeting peptide or protein may be naturally occurring or, alternately, completely or partially synthetic. Exemplary peptides and proteins include ligands and other peptides that bind to cell surface receptors present in liver cells include ligands capable of binding the Sr-B 1 receptor for apoliprotein E, galactose- and lactose-specific lectins, low density lipoprotein receptor ligands, asialoglycoprotein (galactose-terminal) ligands and the like.

Alternatively, the exogenous targeting sequence may be an antibody or an antigen-recognizing moiety thereof. The term “antibody” as used herein refers to all types of immunoglobulins, including IgG, IgM, IgA, IgD, and IgE. The antibodies may be monoclonal or polyclonal and may be of any species of origin, including (for example) mouse, rat, rabbit, horse, or human, or may be chimeric antibodies. Also encompassed by the term “antibody” are bispecific or “bridging” antibodies as known by those skilled in the art. Antibody fragments within the scope of the present invention include, for example, Fab, F(ab′)2, and Fc fragments, and the corresponding fragments obtained from antibodies other than IgG. Such fragments may be produced by known techniques. The exogenous amino acid sequence inserted into the virion capsid may be one that facilitates purification or detection of the virion. For example, the exogenous amino acid sequence may include a poly-histidine sequence that is useful for purifying the virion over a nickel column, as is known to those skilled in the art or an antigenic peptide or protein that may be employed to purify the virion by standard immunopurification techniques. Alternatively, the amino acid sequence may encode a receptor ligand or any other peptide or protein that may be used to purify the modified virion by affinity purification or any other techniques known in the art (e.g., purification techniques based on differential size, density, charge, or isoelectric point, ion-exchange chromatography, or peptide chromatography).

Alternatively, the exogenous targeting sequence may be an antibody or an antigen-recognizing moiety thereof. The term “antibody” as used herein refers to all types of immunoglobulins, including IgG, IgM, IgA, IgD, and IgE. The antibodies may be monoclonal or polyclonal and may be of any species of origin, including (for example) mouse, rat, rabbit, horse, or human, or may be chimeric antibodies. Also encompassed by the term “antibody” are bispecific or “bridging” antibodies as known by those skilled in the art. Antibody fragments within the scope of the present invention include, for example, Fab, F(ab′)2, and Fc fragments, and the corresponding fragments obtained from antibodies other than IgG. Such fragments may be produced by known techniques.

The exogenous amino acid sequence inserted into the virion capsid may be one that facilitates purification or detection of the virion. According to this aspect of the invention, it is not necessary that the exogenous amino acid sequence also alters the virion of the modified parvovirus. For example, the exogenous amino acid sequence may include a poly-histidine sequence that is useful for purifying the virion over a nickel column, as is known to those skilled in the art or an antigenic peptide or protein that may be employed to purify the virion by standard immunopurification techniques. Alternatively, the amino acid sequence may encode a receptor ligand or any other peptide or protein that may be used to purify the modified virion by affinity purification or any other techniques known in the art (e.g., purification techniques based on differential size, density, charge, or isoelectric point, ion-exchange chromatography, or peptide chromatography).

In some embodiments, the AAV vectors disclosed herein may be useful for treating and/or preventing a neurodegenerative, neurodevelopmental, psychiatric, onclological, or autoimune disease or an infection in a subject in need thereof, said method comprising administering to the subject a therapeutically effective amount of any of the above-described rAAV vectors or pharmaceutical compositions comprising such vectors.

### Promoters

In some embodiments, the vector disclosed herein may be a recombinant adeno-associated virus (rAAV) expression vector comprising (i) a capsid protein and (ii) a nucleic acid comprising a promoter operably linked to a nucleotide sequence encoding a deoxyribonuclease (DNase) enzyme. In some embodiments, the promoter is a liver-specific promoter. In some embodiments, the promoter is specific for tumor originator tissue or metastasis target tissue. In some embodiments, the liver-specific promoter is specific for tumor originator tissue or metastasis target tissue.

Non-limiting examples of promoters which may be used in vectors of the present invention, include, e.g., an albumin promoter, an α1-anti-trypsin (AAT) promoter, a thyroid hormone-binding globulin promoter, an alpha fetoprotein promoter, an alcohol dehydrogenase promoter, a factor VIII (FVIII) promoter, a HBV basic core promoter (BCP), a HBV PreS2 promoter, a phosphoenol pyruvate carboxykinase (PEPCK) promoter, a thyroxin-binding globulin (TBG) promoter, an Hepatic Control Region (HCR)-ApoCII hybrid promoter, an HCR-hAAT hybrid promoter, an apolipoprotein E (ApoE) promoter, a low density lipoprotein promoter, a pyruvate kinase promoter, a phosphenol pyruvate carboxykinase promoter, a phenylalanine hydroxylase promoter, a lecithin-cholesterol acyl transferase (LCAT) promoter, an apolipoprotein H (ApoH) promoter, an apolipoprotein A-II promoter (APOA2), a transferrin promoter, a transthyretin promoter, an α-fibrinogen promoter, a β-fibrinogen promoter, an alpha 1-antichymotrypsin promoter, an α2-HS glycoprotein promoter, an haptoglobin promoter, a ceruloplasmin promoter, a plasminogen promoter, a promoter of a complement protein, α1-acid glycoprotein promoter, a LSP1 promoter, a serpin peptidase inhibitor promoter, a Glade A member 1 (SERPINA1) (hAAT) promoter, a Cytochrome P450 family 3 subfamily A polypeptide 4 (CYP3A4) promoter, a microRNA 122 (miR-122) promoter, a liver-specific IGF-II promoter P1, a transthyretin (MTTR) promoter, and an a-fetoprotein (AFP) promoter.

In some embodiments, the promoter is a liver-specific/nervous system-specific tandem promoter. In some embodiments, the liver-specific/nervous system-specific tandem promoter mediates a substantially increased expression of the enzyme in the liver and/or nervous system as compared to other tissues and organs. In some embodiments, the liver-specific/nervous system-specific tandem promoter comprises a liver-specific promoter selected from the group consisting of an albumin promoter, an α1-anti-trypsin (AAT) promoter, a thyroid hormone-binding globulin promoter, an alpha fetoprotein promoter, an alcohol dehydrogenase promoter, a factor VIII (FVIII) promoter, a HBV basic core promoter (BCP), a HBV PreS2 promoter, a phosphoenol pyruvate carboxykinase (PEPCK) promoter, a thyroxin-binding globulin (TBG) promoter, an Hepatic Control Region (HCR)-ApoCII hybrid promoter, an HCR-hAAT hybrid promoter, an apolipoprotein E (ApoE) promoter, a low density lipoprotein promoter, a pyruvate kinase promoter, a phosphenol pyruvate carboxykinase promoter, a phenylalanine hydroxylase promoter, a lecithin-cholesterol acyl transferase (LCAT) promoter, an apolipoprotein H (ApoH) promoter, an apolipoprotein A-II promoter (APOA2), a transferrin promoter, a transthyretin promoter, an a-fibrinogen promoter, a β-fibrinogen promoter, an alpha 1-antichymotrypsin promoter, an α2-HS glycoprotein promoter, an haptoglobin promoter, a ceruloplasmin promoter, a plasminogen promoter, a promoter of a complement protein, al-acid glycoprotein promoter, a LSP1 promoter, a serpin peptidase inhibitor promoter, a Glade A member 1 (SERPINA1) (hAAT) promoter, a Cytochrome P450 family 3 subfamily A polypeptide 4 (CYP3A4) promoter, a microRNA 122 (miR-122) promoter, a liver-specific IGF-II promoter P1, a transthyretin (MTTR) promoter, and an a-fetoprotein (AFP) promoter. In some embodiments, the liver-specific promoter is an anti-trypsin (AAT) promoter. In some embodiments, the AAT promoter is a human al-anti-trypsin (hAAT) promoter. In some embodiments, the liver-specific promoter comprises a sequence having at least 80% or at least 85% or at least 90% or at least 95% sequence identity to the sequence SEQ ID NO: 15. In some embodiments, the liver-specific promoter comprises the sequence SEQ ID NO: 15. In some embodiments, the liver-specific promoter consists of the sequence SEQ ID NO: 15. In some embodiments, the liver-specific promoter is an albumin promoter. In some embodiments, the liver-specific promoter comprises a sequence having at least 80% or at least 85% or at least 90% or at least 95% sequence identity to the sequence SEQ ID NO: 8. In some embodiments, the liver-specific promoter comprises the sequence SEQ ID NO: 8. In some embodiments, the liver-specific promoter consists of the sequence SEQ ID NO: 8. In some embodiments, the liver-specific/nervous system-specific tandem promoter comprises a nervous system-specific promoter selected from the group consisting of F4/80 promoter, CD68 promoter, TMEM119 promoter, CX3CR1 promoter, CMV promoter, MEF2 promoter, FoxP2 promoter, Iba1 promoter, TTR promoter, CD1lb promoter, c-fes promoter, NSE promoter, synapsin promoter, CamKII promoter, α-CaMKII promoter, VGLUT1 promoter, and glial fibrillary acidic protein (GFAP) promoter. In some embodiment, the nervous system-specific promoter is a synapsin promoter. In some embodiments, the synapsin promoter is a human synapsin promoter. In one embodiment, the nervous system-specific promoter is a F4/80 promoter. In some embodiments, the nervous system-specific promoter is a CMV promoter. In some embodiments, the nervous system-specific promoter is a TMEM119 promoter. In some embodiments, the nervous system-specific promoter is a MEF2 promoter. In some embodiments, the nervous system-specific promoter is a FoxP2 promoter. In some embodiments, the liver-specific/nervous system-specific tandem promoter comprises a nervous-specific promoter comprising a sequence having at least 80% or at least 85% or at least 90% or at least 95% sequence identity to any one of the sequences SEQ ID NO: 36 and 38-42. In one embodiment, the nervous system-specific promoter comprises any one of the sequences SEQ ID NO: 36 and 38-42. In one embodiment, the nervous system-specific promoter consists of any one of the sequences SEQ ID NO: 36 and 38-42.

In some embodiments of any of the methods of the invention, the promoter is a liver-specific promoter. In some embodiments of any of the methods of the invention, the liver-specific promoter mediates a substantially increased expression of the enzyme in the liver as compared to other tissues and organs. Non-limiting examples of liver-specific promoters include, e.g., albumin promoter (Alb), human alpha-1 anti-trypsin (hAAT) promoter, thyroxine binding globulin (TBG) promoter, Apolipoprotein E hepatic control region promoter, Apolipoprotein A-II (APOA2) promoter, serpin peptidase inhibitor, Glade A, member 1 (SERPINA1) (hAAT) promoter, cytochrome P450 family 3, subfamily A polypeptide 4 (CYP3A4) promoter, microRNA 122 (miR-122) promoter, Liver-specific IGF-II promoter P1, murine transthyretin (MTTR) promoter, the alpha-fetoprotein (AFP) promoter, a thyroid hormone-binding globulin promoter, an alcohol dehydrogenase promoter, the factor VIII (FVIII) promoter, a HBV basic core promoter (BCP) and PreS2 promoter, a phosphoenol pyruvate carboxykinase (PEPCK) promoter, an Hepatic Control Region (HCR)-ApoCII hybrid promoter, an AAT promoter combined with the mouse albumin gene enhancer (Ealb) element, a low density lipoprotein promoter, a pyruvate kinase promoter, a phosphenol pyruvate carboxykinase promoter, a lecithin-cholesterol acyl transferase (LCAT) promoter, an apolipoprotein H (ApoH) promoter, the transferrin promoter, a transthyretin promoter, an alpha-fibrinogen and beta-fibrinogen promoters, an alpha 1-antichymotrypsin promoter, an alpha 2-HS glycoprotein promoter, an haptoglobin promoter, a ceruloplasmin promoter, a plasminogen promoter, promoters of the complement proteins (e.g., Clq, Clr, C2, C3, C4, C5, C6, C8, C9, complement Factor I, and Factor H), C3 complement activator and the [alpha]1-acid glycoprotein promoter. Additional tissue-specific promoters may be found in the Tissue-Specific Promoter Database, TiProD (Nucleic Acids Research, J4:D104-D107 (2006).

In some embodiments, e.g., when liver targeting is mediated by a capsid protein, a nucleic acid encoding a DNase enzyme can be operably linked to a promoter that allows for efficient systemic expression (e.g., CMV promoter, chicken beta actin promoter (CBA), EFla promoter).

In some embodiments, the liver-specific promoter is α1-anti-trypsin (AAT) promoter. In one specific embodiment, the anti-trypsin promoter is a human α1-anti-trypsin (AAT) promoter. In some embodiments, the promoter is an al-anti-trypsin (AAT) promoter. In some embodiments, the promoter is a human α1-anti-trypsin (hAAT) promoter. In some embodiments, the anti-trypsin promoter comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the sequence of SEQ ID NO: 15. In some embodiments, the AAT promoter comprises the sequence of SEQ ID NO: 15. In some embodiment, the AAT promoter consists of the sequence of SEQ ID NO: 15.

In some embodiments, the promoter region may be operably linked to the coding sequence to cause or improve expression of the protein of interest in transfected cells. Such a promoter may be ubiquitous, cell- or tissue-specific, strong, weak, regulated, chimeric, etc., for example to allow efficient and stable production of the protein in the liver and/or nervous system. The promoter may be homologous to the encoded protein, or heterologous, although generally promoters of use in the disclosed methods are functional in human cells. Examples of regulated promoters include, without limitation, Tet on/off element-containing promoters, rapamycin-inducible promoters, tamoxifen-inducible promoters, and metallothionein promoters.

In some embodiments, the promoters that may be used in the present invention may include promoters that are tissue specific, e.g., for liver and/or nervous system. Non-limiting examples of liver-specific promoters include, e.g., the albumin promoter (Alb), human alpha-1 anti-trypsin (hAAT) promoter, thyroxine binding globulin (TBG), apolipoprotein E hepatic control region promoter, apolipoprotein A-II (APOA2) promoter, serpin peptidase inhibitor, Glade A, member 1 (SERPINA1) (hAAT) promoter, cytochrome P450 family 3, subfamily A polypeptide 4 (CYP3A4) promoter, microRNA 122 (miR-122) promoter, liver-specific IGF-II promoter P1, murine transthyretin (MTTR) promoter, and the alpha-fetoprotein (AFP) promoter. Non-limiting examples of nervous system-specific promoters include, e.g., microglia-specific promoters (e.g., F4/80, CD68, TMEM119, CX3CR1, CMV, Iba1), myeloid-specific promoters (e.g., TTR, CD11b, c-fes), neuron specific promoters (e.g., CMV, NSE, synapsin [SynI, SynII], CamKII, α-CaMKII, VGLUT1), and other neural and glial cell (e.g., oligodendrocytes astrocytes) type-specific promoters (e.g., GFAP). Non-limiting examples of intestine-specific promoters include, MUC2, Villin, T3b, CB/CMV, GFAP, miCMV, CMV+I, tetO-CMV, and β-acti-CMV.

In some embodiments, the promoters that may be used in the present invention may include ubiquitous promoters. Non-limiting examples of ubiquitous promoters include, e.g., viral promoters such as the CMV promoter, the RSV promoter, the SV40 promoter, etc., and cellular promoters such as the phosphoglycerate kinase (PGK) promoter, EF1a promoter, CMVE/CAG promoter system, and the (3-actin promoter.

### DNase Enzyme Administration

In one aspect, the invention provides a method of treating a cancer in a subject in need thereof, comprising administering to the subject a deoxyribonuclease enzyme and at least one cell comprising a chimeric antigen receptor (CAR) or a T cell receptor (TCR).

In a related aspect, the invention provides a method of treating a cancer in a subject in need thereof, comprising administering to the subject a deoxyribonuclease enzyme and at least one cell comprising a chimeric antigen receptor (CAR) or a T cell receptor (TCR), wherein said deoxyribonuclease enzyme is effective to reduce expression of immunosuppressive proteins in tumor tissue and/or increase content of cells comprising a chimeric antigen receptor or T cell receptor within tumor tissue. Non-limiting examples of immunosuppressive proteins include, e.g., PD-L1, PD-L2, CD80, CD86, and MEW.

The administration of a DNase enzyme according to the methods of the invention can be performed by any suitable route, including systemic administration as well as administration directly to the site of the disease (e.g., to a primary tumor). Specific non-limiting examples of useful routes of administration include intravenous (IV), subcutaneous (SC), intraperitoneal (IP), and intramuscular.

In some embodiments of any of the above methods, the deoxyribonuclease enzyme protein is administered via injection. In some embodiments, the deoxyribonuclease enzyme protein is administered via intravenous injection. In some embodiments, the deoxyribonuclease enzyme protein is injected intravenously following infusion of the CAR expressing cells or TCR expressing cells disclosed herein. In some embodiments, the deoxyribonuclease enzyme protein is injected intravenously prior infusion of the CAR expressing cells or TCR expressing cells disclosed herein. In some embodiments, the deoxyribonuclease enzyme protein is injected intravenously together infusion of the CAR expressing cells or TCR expressing cells disclosed herein. In some embodiment the deoxyribonuclease enzyme protein is injected intravenously for at least 14 days following infusion of the CAR expressing cells or TCR expressing cells. In some embodiments, the deoxyribonuclease enzyme protein is injected intravenously for at least 16 days following infusion of the CAR expressing cells or TCR expressing cells. In some embodiments, the deoxyribonuclease enzyme protein is injected intravenously for at least 3 days prior, together or following infusion of the CAR expressing cells or TCR expressing cells. In some embodiments, the deoxyribonuclease enzyme protein is injected intravenously for at least 7 days following infusion of the CAR expressing cells or TCR expressing cells. In some embodiments, the deoxyribonuclease enzyme protein is injected intravenously for at least 7 days prior to infusion of CAR expressing cells or TCR expressing cells. In some embodiments, the deoxyribonuclease enzyme protein is injected intravenously for at least 3 days prior or following infusion of CAR expressing cells or TCR expressing cells. In some embodiments, the deoxyribonuclease enzyme protein is injected intravenously for at least 14 days.

DNase doses useful in the methods of the invention depend on the type of CAR/TCR therapy, the patient's clinical history and response to DNase, as well as the discretion of the attending physician. Non-limiting examples of useful dosage ranges include from 0.5 to 100 mg/kg/day or from 1000 to 200000 KU/kg/day, preferably from 0.5 to 50 mg/kg/day or from 1000 to 100000 Kunitz units (KU)/kg/day, more preferably from 1.5 to 50 mg/kg/day or from 3000 to 100000 KU/kg/day, most preferably from 10 to 50 mg/kg/day or from 20000 to 100000 KU/kg/day.

In some embodiments, the deoxyribonuclease enzyme protein is injected intravenously at 250 μg/kg/day. In some embodiments, the deoxyribonuclease enzyme protein is injected intravenously for at least 14 days at 250 μg/kg/day.

In some embodiments, the CAR comprises a solid tumor associated target antigen binding domain capable of specific binding to one or more antigens selected from BCMA, CD44v6, CAIX (carbonic anhydrase IX), CEA (carcinoembryonic antigen), CDS, CD7, CD19, CD20, CD22, CD30, CD36, CD37, CD70, CD123, CD133, c-Met (Hepatocyte growth factor receptor), EGFR (epidermal growth factor), EGFRvIII (type III variant epidermal growth factor), EGFRB3, Epcam (epithelial cell adhesion molecule), EphA2 (Erythropoeitin producing hepatocellular carcinoma A2), Fetal acetylcholine receptor, FLT3, GD2 (Ganglioside GD2), GPC3 (Glypican-3), GUCY2C (Guanylyl cyclase C), HER1 (human epidermal growth factor receptor 1), HER2 (human epidermal growth factor receptor 2), ICAM-1 (intercellular adhesion molecule 1), IL3R-alpha, IL13Rα2 (Interleukin 13 receptor α2) IL11Rα (interleukin 11 receptor α), Kras (Kristen rat sarcoma viral oncogene homolog) Kras G12D, L1CAM (L1-cell adhesion molecule), MAGE, MET, Mesothelin, MUC (mucin 1), MUC16 ecto (mucin 16), NKG2D (natural killer group 2 member D), NY-ESO-1, PSCA (prostate stem cell antigen), PSMA, ROR1 and WT-1 (Wilms tumor 1).

As used herein, “effective amount” includes a dose suitable for treating a mammal having a clinically defined disorder.

An “analog,” “variant” or “derivative” is a compound substantially similar in structure and having the same biological activity, albeit in certain instances to a differing degree, to a naturally-occurring molecule. For example, a polypeptide variant refers to a polypeptide sharing substantially similar structure and having the same biological activity as a reference polypeptide. Variants or analogs differ in the composition of their amino acid sequences compared to the naturally-occurring polypeptide from which the analog is derived, based on one or more mutations involving (i) deletion of one or more amino acid residues at one or more termini of the polypeptide and/or one or more internal regions of the naturally-occurring polypeptide sequence (e.g., fragments), (ii) insertion or addition of one or more amino acids at one or more termini (typically an “addition” or “fusion”) of the polypeptide and/or one or more internal regions (typically an “insertion”) of the naturally-occurring polypeptide sequence or (iii) substitution of one or more amino acids for other amino acids in the naturally-occurring polypeptide sequence. By way of example, a “derivative” refers to a polypeptide sharing the same or substantially similar structure as a reference polypeptide that has been modified, e.g., chemically.

Variant or analog polypeptides include insertion variants, wherein one or more amino acid residues are added to a protein amino acid sequence of the invention. Insertions may be located at either or both termini of the protein, and/or may be positioned within internal regions of the protein amino acid sequence. Insertion variants, with additional residues at either or both termini, include for example, fusion proteins and proteins including amino acid tags or other amino acid labels. In one aspect, the protein molecule optionally contains an N-terminal Met, especially when the molecule is expressed recombinantly in, for example, a yeast, an insect cell or a bacterial cell such as E. coli.

In deletion variants, one or more amino acid residues in a protein or polypeptide as described herein are removed. Deletions can be affected at one or both termini of the protein or polypeptide, and/or with removal of one or more residues within the protein amino acid sequence. Deletion variants, therefore, include fragments of a protein or polypeptide sequence.

In substitution variants, one or more amino acid residues of a protein or polypeptide are removed and replaced with alternative residues. In one aspect, the substitutions are conservative in nature and conservative substitutions of this type are well known in the art. Alternatively, the invention embraces substitutions that are also non-conservative. Exemplary conservative substitutions are described in Lehninger, [Biochemistry, 2nd Edition; Worth Publishers, Inc., New York (1975), pp.71-77], incorporated by reference herein.

The deoxyribonuclease enzyme protein and/or its derivatives, variants or analogs and/or at least one cell comprising a chimeric antigen receptor (CAR) or a T cell receptor (TCR) may be administered orally, topically, transdermally, parenterally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramuscular, intramammary, intraperitoneal, intrathecal, retrobulbar, intrapulmonary injection and or surgical implantation at a particular site is contemplated as well. Generally, deoxyribonuclease enzyme protein and/or its derivatives, variants or analogs and/or at least one cell comprising a chimeric antigen receptor (CAR) or a T cell receptor (TCR) are essentially free of pyrogens, as well as other impurities that could be harmful to the recipient.

Single or multiple administrations of the deoxyribonuclease enzyme protein and/or its derivatives, variants or analogs and/or at least one cell comprising a chimeric antigen receptor (CAR) or a T cell receptor (TCR) can be carried out with the dose levels and pattern being selected by the treating physician. For the prevention or treatment of cancer, the appropriate dosage will depend on the type of disease to be treated, as described above, the severity and course of the cancer, whether drug is administered for preventive or therapeutic purposes, previous therapy, the patient's clinical history and response to the drug, and the discretion of the attending physician.

The present invention also relates to a deoxyribonuclease enzyme protein and/or its derivatives, variants or analogs and/or at least one cell comprising a chimeric antigen receptor (CAR) or a T cell receptor (TCR) comprising a pharmaceutically acceptable carrier, diluent, salt, buffer, or excipient. The pharmaceutical composition can be used for treating clinically-defined disorders. The pharmaceutical composition of the invention may be a solution or a lyophilized product. Solutions of the pharmaceutical composition may be subjected to any suitable lyophilization process.

In other aspects of this embodiment, a deoxyribonuclease enzyme protein and/or its derivatives, variants or analogs disclosed herein reduces the severity of a cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, a deoxyribonuclease enzyme protein disclosed herein reduces the size of a tumor from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In aspects of this embodiment, a deoxyribonuclease enzyme protein and/or its derivatives, variants or analogs disclosed herein may be at a dose of , e.g., at least 25 μg/kg, at least 50 μg/kg, at least 75 μg/kg, at least 100 μg/kg, at least 125 μg/kg, at least 150 μg/kg, at least 175 μg/kg, at least 200 μg/kg, at least 225 μg/kg, at least 250 μg/kg, at least 275 μg/kg, at least 300 μg/kg, at least 325 μg/kg, at least 350 μg/kg, at least 375 μg/kg, at least 400 μg/kg, at least 425 μg/kg, at least 450 μg/kg, at least 475 μg/kg, at least 500 μg/kg, at least 525 μg/kg, at least 550 μg/kg, at least 575 μg/kg, at least 600 μg/kg, at least 625 μg/kg, at least 650 μg/kg, at least 675 μg/kg, at least 700 μg/kg, at least 725 μg/kg, at least 750 μg/kg, at least 775 μg/kg, at least 800 μg/kg, at least 825 μg/kg, at least 850 μg/kg, at least 875 μg/kg, at least 900 μg/kg, at least 925 μg/kg, at least 950 μg/kg, at least 975 μg/kg, at least 1 mg/kg, at least 1.25 mg/kg, at least 1.5 mg/kg, at least 2 mg/kg, at least 2.5 mg/kg, at least 3 mg/kg, at least 3.5 mg/kg, at least 4 mg/kg, at least 4.5 mg/kg, at least 5 mg/kg, at least 5.5 mg/kg, at least 6 mg/kg, at least 6.5 mg/kg, at least 7 mg/kg, at least 7.5 mg/kg, at least 8 mg/kg, at least 8.5 mg/kg, at least 9 mg/kg, at least 9.5 mg/kg, at least 10 mg/kg, at least 10.5 mg/kg, at least 11 mg/kg, at least 11.5 mg/kg, at least 12 mg/kg, at least 12.5 mg/kg, at least 13 mg/kg, at least 13.5 mg/kg, at least 14 mg/kg, at least 14.5 mg/kg, at least 15 mg/kg, at least 15.5 mg/kg, at least 16 mg/kg, at least 16.5 mg/kg, at least 17 mg/kg, at least 17.5 mg/kg, at least 18 mg/kg, at least 18.5 mg/kg, at least 19 mg/kg, at least 19.5 mg/kg, at least 20 mg/kg, at least 20.5 mg/kg, at least 21 mg/kg, at least 21.5 mg/kg, at least 22 mg/kg, at least 22.5 mg/kg, at least 23 mg/kg, at least 23.5 mg/kg, at least 24 mg/kg, at least 24.5 mg/kg, at least 25 mg/kg, at least 25.5 mg/kg, at least 26 mg/kg, at least 26.5 mg/kg, at least 27 mg/kg, at least 27.5 mg/kg, at least 28 mg/kg, at least 28.5 mg/kg, at least 29 mg/kg, at least 29.5 mg/kg, at least 30 mg/kg, at least 31 mg/kg, at least 32 mg/kg, at least 33 mg/kg, at least 34 mg/kg, at least 35 mg/kg, 36 mg/kg, at least 37 mg/kg, at least 38 mg/kg, at least 39 mg/kg, at least 40 mg/kg, 41 mg/kg, at least 42 mg/kg, at least 43 mg/kg, at least 44 mg/kg, at least 45 mg/kg, at least 46 mg/kg, at least mg/kg, at least 48 mg/kg, at least 49 mg/kg at least 50 mg/kg, at least 51 mg/kg, at least 52 mg/kg, at least 53 mg/kg, at least 54 mg/kg, at least 55 mg/kg, at least 56 mg/kg, at least 57 mg/kg, at least 58 mg/kg, at least 59 mg/kg, at least 60 mg/kg, at least 61 mg/kg, at least 62 mg/kg, at least 63 mg/kg, at least 64 mg/kg, at least 65 mg/kg, at least 66 mg/kg, at least 67 mg/kg, at least 68 mg/kg, at least 69 mg/kg, at least 70 mg/kg, at least 71 mg/kg, at least 72 mg/kg, at least 73 mg/kg, at least 74 mg/kg, at least 75 mg/kg, at least 76 mg/kg, at least 77 mg/kg, at least 78 mg/kg, at least 79 mg/kg, at least 80 mg/kg, 81 mg/kg, at least 82 mg/kg, at least 83 mg/kg, at least 84 mg/kg, at least 85 mg/kg, at least 86 mg/kg, at least 87 mg/kg, at least 88 mg/kg, at least 89 mg/kg, at least 90 mg/kg, at least 91 mg/kg, at least 92 mg/kg, at least 93 mg/kg, at least 94 mg/kg, at least 95 mg/kg, at least 96 mg/kg, at least 97 mg/kg, at least 98 mg/kg, at least 99 mg/kg, at least 100 mg/kg, at least 150 mg/kg, at least 200 mg/kg, at least 250 mg/kg, at least 300 mg/kg, at least 350 mg/kg, at least 400 mg/kg, at least 450 mg/kg, at least 500 mg/kg, at least 550 mg/kg, at least 600 mg/kg, at least 650 mg/kg, at least 700 mg/kg, at least 750 mg/kg, at least 800 mg/kg, at least 850 mg/kg, at least 900 mg/kg, at least 950 mg/kg, at least 1 g/kg, at least 2 g/kg, at least 3 g/kg, at least 4 g/kg, at least 5 g/kg, at least 6 g/kg, at least 7 g/kg, at least 8 g/kg, at least 9 g/kg, at least 10 g/kg, at least 15 g/kg, at least 20 g/kg, at least 25 g/kg, at least 50 g/kg, at least 100 g/kg.

In aspects of this embodiment, a deoxyribonuclease enzyme protein and/or its derivatives, variants or analogs disclosed herein may be at a dose of, e.g., about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 1.25 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, about 10 mg/kg, about 10.5 mg/kg, about 11 mg/kg, about 11.5 mg/kg, about 12 mg/kg, about 12.5 mg/kg, about 13 mg/kg, about 13.5 mg/kg, about 14 mg/kg, about 14.5mg/kg, about 15 mg/kg, about 15.5 mg/kg, about 16 mg/kg, about 16.5 mg/kg, about 17 mg/kg, about 17.5 mg/kg, about 18 mg/kg, about 18.5 mg/kg, about 19 mg/kg, about 19.5 mg/kg, about 20 mg/kg, about 20.5 mg/kg, about 21 mg/kg, about 21.5 mg/kg, about 22 mg/kg, about 22.5 mg/kg, about 23 mg/kg, about 23.5 mg/kg, about 24 mg/kg, about 24.5 mg/kg, about 25 mg/kg, about 25.5 mg/kg, about 26 mg/kg, about 26.5 mg/kg, about 27 mg/kg, about 27.5 mg/kg, about 28 mg/kg, about 28.5 mg/kg, about 29 mg/kg, about 29.5 mg/kg, about 30 mg/kg, about 31 mg/kg, about 32 mg/kg, about 33 mg/kg, about 34 mg/kg, about 35 mg/kg, 36 mg/kg, about 37 mg/kg, about 38 mg/kg, about 39 mg/kg, about 40 mg/kg, 41 mg/kg, about 42 mg/kg, about 43 mg/kg, about 44 mg/kg, about 45 mg/kg, about 46 mg/kg, about mg/kg, about 48 mg/kg, about 49 mg/kg about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, about 100 mg/kg, about 150 mg/kg, about 200 mg/kg, about 250 mg/kg, about 300 mg/kg, about 350 mg/kg, about 400 mg/kg, about 450 mg/kg, about 500 mg/kg, about 550 mg/kg, about 600 mg/kg, about 650 mg/kg, about 700 mg/kg, about 750 mg/kg, about 800 mg/kg, about 850 mg/kg, about 900 mg/kg, about 950 mg/kg, about 1 g/kg, about 2 g/kg, about 3 g/kg, about 4 g/kg, about 5 g/kg, about 6 g/kg, about 7 g/kg, about 8 g/kg, about 9 g/kg, about 10 g/kg, about 15 g/kg, about 20 g/kg, about 25 g/kg, about 50 g/kg, about 100 g/kg.

In aspects of this embodiment, a deoxyribonuclease enzyme and/or its derivatives, variants or analogs disclosed herein may be at a dose of, e.g., no more than 25 μg/kg, no more than 50 μg/kg, no more than 75 μg/kg, no more than 100 μg/kg, no more than 125 μg/kg, no more than 150 μg/kg, no more than 175 μg/kg, no more than 200 μg/kg, no more than 225 μg/kg, no more than 250 μg/kg, no more than 275 μg/kg, no more than 300 μg/kg, no more than 325 μg/kg, no more than 350 μg/kg, no more than 375 μg/kg, no more than 400 μg/kg, no more than 425 μg/kg, no more than 450 μg/kg, no more than 475 μg/kg, no more than 500 μg/kg, no more than 525 μg/kg, no more than 550 μg/kg, no more than 575 μg/kg, no more than 600 μg/kg, no more than 625 μg/kg, no more than 650 μg/kg, no more than 675 μg/kg, no more than 700 μg/kg, no more than 725 μg/kg, no more than 750 μg/kg, no more than 775 μg/kg, no more than 800 μg/kg, no more than 825 μg/kg, no more than 850 μg/kg, no more than 875 μg/kg, no more than 900 μg/kg, no more than 925 μg/kg, no more than 950 μg/kg, no more than 975 μg/kg, no more than 1 mg/kg, no more than 1.25 mg/kg, no more than 1.5 mg/kg, no more than 2 mg/kg, no more than 2.5 mg/kg, no more than 3 mg/kg, no more than 3.5 mg/kg, no more than 4 mg/kg, no more than 4.5 mg/kg, no more than 5 mg/kg, no more than 5.5 mg/kg, no more than 6 mg/kg, no more than 6.5 mg/kg, no more than 7 mg/kg, no more than 7.5 mg/kg, no more than 8 mg/kg, no more than 8.5 mg/kg, no more than 9 mg/kg, no more than 9.5 mg/kg, no more than 10 mg/kg, no more than 10.5 mg/kg, no more than 11 mg/kg, no more than 11.5 mg/kg, no more than 12 mg/kg, no more than 12.5 mg/kg, no more than 13 mg/kg, no more than 13.5 mg/kg, no more than 14 mg/kg, no more than 14.5mg/kg, no more than 15 mg/kg, no more than 15.5 mg/kg, no more than 16 mg/kg, no more than 16.5 mg/kg, no more than 17 mg/kg, no more than 17.5 mg/kg, no more than 18 mg/kg, no more than 18.5 mg/kg, no more than 19 mg/kg, no more than 19.5 mg/kg, no more than 20 mg/kg, no more than 20.5 mg/kg, no more than 21 mg/kg, no more than 21.5 mg/kg, no more than 22 mg/kg, no more than 22.5 mg/kg, no more than 23 mg/kg, no more than 23.5 mg/kg, no more than 24 mg/kg, no more than 24.5 mg/kg, no more than 25 mg/kg, no more than 25.5 mg/kg, no more than 26 mg/kg, no more than 26.5 mg/kg, no more than 27 mg/kg, no more than 27.5 mg/kg, no more than 28 mg/kg, no more than 28.5 mg/kg, no more than 29 mg/kg, no more than 29.5 mg/kg, no more than 30 mg/kg, no more than 31 mg/kg, no more than 32 mg/kg, no more than 33 mg/kg, no more than 34 mg/kg, no more than 35 mg/kg, 36 mg/kg, no more than 37 mg/kg, no more than 38 mg/kg, no more than 39 mg/kg, no more than 40 mg/kg, 41 mg/kg, no more than 42 mg/kg, no more than 43 mg/kg, no more than 44 mg/kg, no more than 45 mg/kg, no more than 46 mg/kg, no more than mg/kg, no more than 48 mg/kg, no more than 49 mg/kg no more than 50 mg/kg, no more than 51 mg/kg, no more than 52 mg/kg, no more than 53 mg/kg, no more than 54 mg/kg, no more than 55 mg/kg, no more than 56 mg/kg, no more than 57 mg/kg, no more than 58 mg/kg, no more than 59 mg/kg, no more than 60 mg/kg, no more than 61 mg/kg, no more than 62 mg/kg, no more than 63 mg/kg, no more than 64 mg/kg, no more than 65 mg/kg, no more than 66 mg/kg, no more than 67 mg/kg, no more than 68 mg/kg, no more than 69 mg/kg, no more than 70 mg/kg, no more than 71 mg/kg, no more than 72 mg/kg, no more than 73 mg/kg, no more than 74 mg/kg, no more than 75 mg/kg, no more than 76 mg/kg, no more than 77 mg/kg, no more than 78 mg/kg, no more than 79 mg/kg, no more than 80 mg/kg, 81 mg/kg, no more than 82 mg/kg, no more than 83 mg/kg, no more than 84 mg/kg, no more than 85 mg/kg, no more than 86 mg/kg, no more than 87 mg/kg, no more than 88 mg/kg, no more than 89 mg/kg, no more than 90 mg/kg, no more than 91 mg/kg, no more than 92 mg/kg, no more than 93 mg/kg, no more than 94 mg/kg, no more than 95 mg/kg, no more than 96 mg/kg, no more than 97 mg/kg, no more than 98 mg/kg, no more than 99 mg/kg, no more than 100 mg/kg, no more than 150 mg/kg, no more than 200 mg/kg, no more than 250 mg/kg, no more than 300 mg/kg, no more than 350 mg/kg, no more than 400 mg/kg, no more than 450 mg/kg, no more than 500 mg/kg, no more than 550 mg/kg, no more than 600 mg/kg, no more than 650 mg/kg, no more than 700 mg/kg, no more than 750 mg/kg, no more than 800 mg/kg, no more than 850 mg/kg, no more than 900 mg/kg, no more than 950 mg/kg, no more than 1 g/kg, no more than 2 g/kg, no more than 3 g/kg, no more than 4 g/kg, no more than 5 g/kg, no more than 6 g/kg, no more than 7 g/kg, no more than 8 g/kg, no more than 9 g/kg, no more than 10 g/kg, no more than 15 g/kg, no more than 20 g/kg, no more than 25 g/kg, no more than 50 g/kg, no more than 100 g/kg.

In yet other aspects of this embodiment, a deoxyribonuclease enzyme protein and/or its derivatives, variants or analogs disclosed herein may be in the dose range of, e.g., about 1 μg/kg to about 30 mg/kg, about 5 μg/kg to about 50 mg/kg, about 5 μg/kg to about 75 μg/kg, about 100 μg/kg to about 40 mg/kg, about 500 μg/kg to about 35 mg/kg, about 100 μg/kg to about 30 mg/kg, about 150 μg/kg to about 35 mg/kg, about 250 μg/kg to about 25 mg/kg, about 350 μg/kg to about 30 mg/kg, about 500 μg/kg to about 25 mg/kg, about 150 μg/kg to about 30 mg/kg, about 750 μg/kg to about 30 mg/kg, about 500 μg/kg to about 12 mg/kg, about 200 μg/kg to about 30 mg/kg, about 250 μg/kg to about 40 mg/kg, about 100 μg/kg to about 10 mg/kg, about 200 μg/kg to about 35 mg/kg, about 500 μg/kg to about 30 mg/kg, or about 10 μg/kg to about 30 mg/kg, about 1 mg/kg to about 30 mg/kg, about 5 mg/kg to about 50 mg/kg, about 5 mg/kg to about 75 mg/kg, about 10 mg/kg to about 40 mg/kg, about 5 mg/kg to about 35 mg/kg, about 10 mg/kg to about 30 mg/kg, about 15 mg/kg to about 35 mg/kg, about 25 mg/kg to about 50 mg/kg, about 35 mg/kg to about 60 mg/kg, about 50 mg/kg to about 75 mg/kg, about 15 mg/kg to about 60 mg/kg, about 150 mg/kg to about 100 mg/kg, about 5 mg/kg to about 120 mg/kg, about 20 mg/kg to about 60 mg/kg, about 25 mg/kg to about 75 mg/kg, about 10 mg/kg to about 35 mg/kg, about 20 mg/kg to about 35 mg/kg, about 15 mg/kg to about 30 mg/kg, or about 10 mg/kg to about 30 mg/kg.

In aspects of this embodiment, a therapeutically effective amount of a deoxyribonuclease enzyme protein and/or its derivatives, variants or analogs disclosed herein reduces a symptom associated with a cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In other aspects of this embodiment, a therapeutically effective amount of a deoxyribonuclease enzyme protein and/or its derivatives, variants or analogs disclosed herein reduces a symptom associated with a cancer by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%.

In yet other aspects of this embodiment, a therapeutically effective amount of a deoxyribonuclease enzyme protein and/or its derivatives, variants or analogs disclosed herein reduces a symptom associated with a cancer by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In one embodiment, a deoxyribonuclease enzyme protein and/or its derivatives, variants or analogs disclosed herein is capable of reducing the severity of a disease in an individual suffering from a cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment.

In other aspects of this embodiment, a deoxyribonuclease enzyme protein and/or its derivatives, variants or analogs is capable of reducing the severity of a disease in an individual suffering from a cancer by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In aspects of this embodiment, a therapeutically effective amount of a deoxyribonuclease enzyme protein and/or its derivatives, variants or analogs and at least one cell comprising a chimeric antigen receptor (CAR) or a T cell receptor (TCR) disclosed herein reduces a symptom associated with a cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In other aspects of this embodiment, a therapeutically effective amount of a deoxyribonuclease enzyme protein and/or its derivatives, variants or analogs and at least one cell comprising a chimeric antigen receptor (CAR) or a T cell receptor (TCR) disclosed herein reduces a symptom associated with a cancer by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%.

In yet other aspects of this embodiment, a therapeutically effective amount of a deoxyribonuclease enzyme protein and/or its derivatives, variants or analogs and at least one cell comprising a chimeric antigen receptor (CAR) or a T cell receptor (TCR) disclosed herein reduces a symptom associated with a cancer by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In one embodiment, a deoxyribonuclease enzyme protein and/or its derivatives, variants or analogs and at least one cell comprising a chimeric antigen receptor (CAR) or a T cell receptor (TCR) disclosed herein is capable of reducing the severity of a disease in an individual suffering from a cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment.

In other aspects of this embodiment, a deoxyribonuclease enzyme protein and/or its derivatives, variants or analogs and at least one cell comprising a chimeric antigen receptor (CAR) or a T cell receptor (TCR) is capable of reducing the severity of a disease in an individual suffering from a cancer by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In a further embodiment, a deoxyribonuclease enzyme protein and/or its derivatives, variants or analogs are administered one or more time every 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more after the first administration of a deoxyribonuclease enzyme protein.

In a further embodiment, a deoxyribonuclease enzyme protein and/or its derivatives, variants or analogs and at least one cell comprising a chimeric antigen receptor (CAR) or a T cell receptor (TCR) are administered one or more time every 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 1 week, 2 weeks, 3 weeks, 4 weeks, one month, two months, three months, four months or more after the first administration of a deoxyribonuclease enzyme protein and/or its derivatives, variants or analogs and at least one cell comprising a chimeric antigen receptor (CAR) or a T cell receptor (TCR).

In an embodiment, the period of administration of a deoxyribonuclease enzyme protein and/or its derivatives, variants or analogs is for 10 minutes, 20 minutes, 30 minutes, 45 minutes, 1 hour, 2 hours, 3 hours, 4 hours, 5 hours, 6 hours, 7 hours, 8 hours, 9 hours, 10 hours 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In an embodiment, the period of administration of a deoxyribonuclease enzyme protein and/or its derivatives, variants or analogs and at least one cell comprising a chimeric antigen receptor (CAR) or a T cell receptor (TCR) is for about 10 minutes, about 20 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 4 months, about 5 months, about 6 months, about 7 months, about 8 months, about 9 months, about 10 months, about 11 months, about 12 months, or more.

In an embodiment, the period of administration of a deoxyribonuclease enzyme protein and/or its derivatives, variants or analogs is for at least 10 minutes, at least 20 minutes, at least 30 minutes, at least 45 minutes, at least 1 hour, at least 2 hours, at least 3 hours, at least 4 hours, at least 5 hours, at least 6 hours, at least 7 hours, at least 8 hours, at least 9 hours, at least 10 hours at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 4 months, at least 5 months, at least 6 months, at least 7 months, at least 8 months, at least 9 months, at least 10 months, at least 11 months, at least 12 months, or more.

In an embodiment, the period of administration of a deoxyribonuclease enzyme protein and/or its derivatives, variants or analogs is for no more than 10 minutes, no more than 20 minutes, no more than 30 minutes, no more than 45 minutes, no more than 1 hour, no more than 2 hours, no more than 3 hours, no more than 4 hours, no more than 5 hours, no more than 6 hours, no more than 7 hours, no more than 8 hours, no more than 9 hours, no more than 10 hours no more than 11 hours, no more than 12 hours, no more than 13 hours, no more than 14 hours, no more than 15 hours, no more than 16 hours, no more than 17 hours, no more than 18 hours, no more than 19 hours, no more than 20 hours, no more than 21 hours, no more than 22 hours, no more than 23 hours, no more than 1 day, no more than 2 days, no more than 3 days, no more than 4 days, no more than 5 days, no more than 6 days, no more than 7 days, no more than 8 days, no more than 9 days, no more than 10 days, no more than 11 days, no more than 12 days, no more than 13 days, no more than 14 days, no more than 3 weeks, no more than 4 weeks, no more than 5 weeks, no more than 6 weeks, no more than 7 weeks, no more than 8 weeks, no more than 9 weeks, no more than 10 weeks, no more than 11 weeks, no more than 12 weeks, no more than 4 months, no more than 5 months, no more than 6 months, no more than 7 months, no more than 8 months, no more than 9 months, no more than 10 months, no more than 11 months, no more than 12 months, or more.

In a further embodiment, a period of administration of a deoxyribonuclease enzyme protein and/or its derivatives, variants or analogs is stopped is for 10 minutes, 20 minutes, 30 minutes, 45 minutes, 1 hour, 2 hours, 3 hours, 4 hours, 5 hours, 6 hours, 7 hours, 8 hours, 9 hours, 10 hours 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In a further embodiment, a period of administration of a deoxyribonuclease enzyme protein and/or its derivatives, variants or analogs is stopped is for about 10 minutes, about 20 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 4 months, about 5 months, about 6 months, about 7 months, about 8 months, about 9 months, about 10 months, about 11 months, about 12 months, or more.

In a further embodiment, a administration of a deoxyribonuclease enzyme protein and/or its derivatives, variants or analogs is stopped is for no more than 10 minutes, no more than 20 minutes, no more than 30 minutes, no more than 45 minutes, no more than 1 hour, no more than 2 hours, no more than 3 hours, no more than 4 hours, no more than 5 hours, no more than 6 hours, no more than 7 hours, no more than 8 hours, no more than 9 hours, no more than 10 hours, no more than 11 hours, no more than 12 hours, no more than 13 hours, no more than 14 hours, no more than 15 hours, no more than 16 hours, no more than 17 hours, no more than 18 hours, no more than 19 hours, no more than 20 hours, no more than 21 hours, no more than 22 hours, no more than 23 hours, no more than 1 day, no more than 2 days, no more than 3 days, no more than 4 days, no more than 5 days, no more than 6 days, no more than 7 days, no more than 8 days, no more than 9 days, no more than 10 days, no more than 11 days, no more than 12 days, no more than 13 days, no more than 14 days, no more than 3 weeks, No more than 4 weeks, no more than 5 weeks, no more than 6 weeks, no more than 7 weeks, no more than 8 weeks, no more than 9 weeks, no more than 10 weeks, no more than 11 weeks, no more than 12 weeks, no more than 4 months, no more than 5 months, no more than 6 months, no more than 7 months, no more than 8 months, no more than 9 months, no more than 10 months, no more than 11 months, no more than 12 months, or more.

In a further embodiment, a administration of a deoxyribonuclease enzyme protein and/or its derivatives, variants or analogs is stopped is for at least 10 minutes, at least 20 minutes, at least 30 minutes, at least 45 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 4 months, at least 5 months, at least 6 months, at least 7 months, at least 8 months, at least 9 months, at least 10 months, at least 11 months, at least 12 months, or more.

In an embodiment, the period of administration of a deoxyribonuclease enzyme protein and/or its derivatives, variants or analogs and/or at least one cell comprising a chimeric antigen receptor (CAR) or a T cell receptor (TCR) is for 10 minutes, 20 minutes, 30 minutes, 45 minutes, 1 hour, 2 hours, 3 hours, 4 hours, 5 hours, 6 hours, 7 hours, 8 hours, 9 hours, 10 hours 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In an embodiment, the period of administration of a deoxyribonuclease enzyme protein and/or its derivatives, variants or analogs and/or at least one cell comprising a chimeric antigen receptor (CAR) or a T cell receptor (TCR) is for about 10 minutes, about 20 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 4 months, about 5 months, about 6 months, about 7 months, about 8 months, about 9 months, about 10 months, about 11 months, about 12 months, or more.

In an embodiment, the period of administration of a deoxyribonuclease enzyme protein and/or its derivatives, variants or analogs and/or at least one cell comprising a chimeric antigen receptor (CAR) or a T cell receptor (TCR) is for at least 10 minutes, at least 20 minutes, at least 30 minutes, at least 45 minutes, at least 1 hour, at least 2 hours, at least 3 hours, at least 4 hours, at least 5 hours, at least 6 hours, at least 7 hours, at least 8 hours, at least 9 hours, at least 10 hours at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 4 months, at least 5 months, at least 6 months, at least 7 months, at least 8 months, at least 9 months, at least 10 months, at least 11 months, at least 12 months, or more.

In an embodiment, the period of administration of a deoxyribonuclease enzyme protein and/or its derivatives, variants or analogs and/or at least one cell comprising a chimeric antigen receptor (CAR) or a T cell receptor (TCR) is for no more than 10 minutes, no more than 20 minutes, no more than 30 minutes, no more than 45 minutes, no more than 1 hour, no more than 2 hours, no more than 3 hours, no more than 4 hours, no more than 5 hours, no more than 6 hours, no more than 7 hours, no more than 8 hours, no more than 9 hours, no more than 10 hours no more than 11 hours, no more than 12 hours, no more than 13 hours, no more than 14 hours, no more than 15 hours, no more than 16 hours, no more than 17 hours, no more than 18 hours, no more than 19 hours, no more than 20 hours, no more than 21 hours, no more than 22 hours, no more than 23 hours, no more than 1 day, no more than 2 days, no more than 3 days, no more than 4 days, no more than 5 days, no more than 6 days, no more than 7 days, no more than 8 days, no more than 9 days, no more than 10 days, no more than 11 days, no more than 12 days, no more than 13 days, no more than 14 days, no more than 3 weeks, no more than 4 weeks, no more than 5 weeks, no more than 6 weeks, no more than 7 weeks, no more than 8 weeks, no more than 9 weeks, no more than 10 weeks, no more than 11 weeks, no more than 12 weeks, no more than 4 months, no more than 5 months, no more than 6 months, no more than 7 months, no more than 8 months, no more than 9 months, no more than 10 months, no more than 11 months, no more than 12 months, or more.

In a further embodiment, a period of administration of a deoxyribonuclease enzyme protein and/or its derivatives, variants or analogs and/or at least one cell comprising a chimeric antigen receptor (CAR) or a T cell receptor (TCR) during which administration is stopped is for 10 minutes, 20 minutes, 30 minutes, 45 minutes, 1 hour, 2 hours, 3 hours, 4 hours, 5 hours, 6 hours, 7 hours, 8 hours, 9 hours, 10 hours 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In an embodiment, the period of administration of a deoxyribonuclease enzyme protein and/or its derivatives, variants or analogs and/or at least one cell comprising a chimeric antigen receptor (CAR) or a T cell receptor (TCR) is for no more than 10 minutes, no more than 20 minutes, no more than 30 minutes, no more than 45 minutes, no more than 1 hour, no more than 2 hours, no more than 3 hours, no more than 4 hours, no more than 5 hours, no more than 6 hours, no more than 7 hours, no more than 8 hours, no more than 9 hours, no more than 10 hours, no more than 11 hours, no more than 12 hours, no more than 13 hours, no more than 14 hours, no more than 15 hours, no more than 16 hours, no more than 17 hours, no more than 18 hours, no more than 19 hours, no more than 20 hours, no more than 21 hours, no more than 22 hours, no more than 23 hours, no more than 1 day, no more than 2 days, no more than 3 days, no more than 4 days, no more than 5 days, no more than 6 days, no more than 7 days, no more than 8 days, no more than 9 days, no more than 10 days, no more than 11 days, no more than 12 days, no more than 13 days, no more than 14 days, no more than 3 weeks, no more than 4 weeks, no more than 5 weeks, no more than 6 weeks, no more than 7 weeks, no more than 8 weeks, no more than 9 weeks, no more than 10 weeks, no more than 11 weeks, no more than 12 weeks, no more than 4 months, no more than 5 months, no more than 6 months, no more than 7 months, no more than 8 months, no more than 9 months, no more than 10 months, no more than 11 months, no more than 12 months, or more.

In a further embodiment, a period during which administration of a deoxyribonuclease enzyme protein and/or its derivatives, variants or analogs and/or at least one cell comprising a chimeric antigen receptor (CAR) or a T cell receptor (TCR)is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In an embodiment, the period of administration of a deoxyribonuclease enzyme protein and/or its derivatives, variants or analogs and/or at least one cell comprising a chimeric antigen receptor (CAR) or a T cell receptor (TCR) is for at least 10 minutes, at least 20 minutes, at least 30 minutes, at least 45 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 4 months, at least 5 months, at least 6 months, at least 7 months, at least 8 months, at least 9 months, at least 10 months, at least 11 months, at least 12 months, or more.

In a further embodiment, a period of administration of a deoxyribonuclease enzyme protein and/or its derivatives, variants or analogs and/or at least one cell comprising a chimeric antigen receptor (CAR) or a T cell receptor (TCR) during which administration is stopped is for 10 minutes, 20 minutes, 30 minutes, 45 minutes, 1 hour, 2 hours, 3 hours, 4 hours, 5 hours, 6 hours, 7 hours, 8 hours, 9 hours, 10 hours 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In a further embodiment, a period of administration of a deoxyribonuclease enzyme protein and/or its derivatives, variants or analogs and/or at least one cell comprising a chimeric antigen receptor (CAR) or a T cell receptor (TCR) during which administration is stopped for at least 10 minutes, at least 20 minutes, at least 30 minutes, at least 45 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 4 months, at least 5 months, at least 6 months, at least 7 months, at least 8 months, at least 9 months, at least 10 months, at least 11 months, at least 12 months, or more.

In a further embodiment, a period of administration of a deoxyribonuclease enzyme protein and/or its derivatives, variants or analogs and/or at least one cell comprising a chimeric antigen receptor (CAR) or a T cell receptor (TCR) during which administration is stopped for no more than 10 minutes, no more than 20 minutes, no more than 30 minutes, no more than 45 minutes, no more than 1 hour, no more than 2 hours, no more than 3 hours, no more than 4 hours, no more than 5 hours, no more than 6 hours, no more than 7 hours, no more than 8 hours, no more than 9 hours, no more than 10 hours, no more than 11 hours, no more than 12 hours, no more than 13 hours, no more than 14 hours, no more than 15 hours, no more than 16 hours, no more than 17 hours, no more than 18 hours, no more than 19 hours, no more than 20 hours, no more than 21 hours, no more than 22 hours, no more than 23 hours, no more than 1 day, no more than 2 days, no more than 3 days, no more than 4 days, no more than 5 days, no more than 6 days, no more than 7 days, no more than 8 days, no more than 9 days, no more than 10 days, no more than 11 days, no more than 12 days, no more than 13 days, no more than 14 days, no more than 3 weeks, no more than 4 weeks, no more than 5 weeks, no more than 6 weeks, no more than 7 weeks, no more than 8 weeks, no more than 9 weeks, no more than 10 weeks, no more than 11 weeks, no more than 12 weeks, no more than 4 months, no more than 5 months, no more than 6 months, no more than 7 months, no more than 8 months, no more than 9 months, no more than 10 months, no more than 11 months, no more than 12 months, or more.

In a further embodiment, a period of administration of a deoxyribonuclease enzyme protein and/or its derivatives, variants or analogs and/or at least one cell comprising a chimeric antigen receptor (CAR) or a T cell receptor (TCR) during which administration is stopped for about 10 minutes, about 20 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 4 months, about 5 months, about 6 months, about 7 months, about 8 months, about 9 months, about 10 months, about 11 months, about 12 months, or more.

In aspects of this embodiment, a therapeutically effective amount of a deoxyribonuclease enzyme protein and/or its derivatives, variants or analogs disclosed herein reduces or maintains the severity of a cancer in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a deoxyribonuclease enzyme protein and/or its derivatives, variants or analogs disclosed herein reduces or maintains the severity of a cancer in an individual by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a deoxyribonuclease enzyme protein and/or its derivatives, variants or analogs disclosed herein reduces or maintains the severity of a disease in an individual by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In aspects of this embodiment, a therapeutically effective amount of a deoxyribonuclease enzyme protein and/or its derivatives, variants or analogs and at least one cell comprising a chimeric antigen receptor (CAR) or a T cell receptor (TCR) disclosed herein reduces or maintains the severity of a cancer in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a deoxyribonuclease enzyme protein and/or its derivatives, variants or analogs and at least one cell comprising a chimeric antigen receptor (CAR) or a T cell receptor (TCR) disclosed herein reduces or maintains the severity of a cancer in an individual by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a deoxyribonuclease enzyme protein and/or its derivatives, variants or analogs and at least one cell comprising a chimeric antigen receptor (CAR) or a T cell receptor (TCR) disclosed herein reduces or maintains the severity of a disease in an individual by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

A deoxyribonuclease enzyme protein and/or its derivatives, variants or analogs and at least one cell comprising a chimeric antigen receptor (CAR) or a T cell receptor (TCR) is administered to an individual. An individual is typically a human being, but can be an animal, including, but not limited to, dogs, cats, birds, cattle, horses, sheep, goats, reptiles and other animals, whether domesticated or not. Typically, any individual who is a candidate for treatment is a candidate with some form of therapy for a disease the individual is suffering, whether the disease is benign or malignant. With regard to cancer's, the most common types of cancer include, but are not limited to, bladder cancer, breast cancer, colon and rectal cancer, endometrial cancer, kidney cancer, renal cancer, leukemia, lung cancer, melanoma, non-Hodgkins lymphoma, pancreatic cancer, prostate cancer, stomach cancer and thyroid cancer. Pre-operative evaluation typically includes routine history and physical examination in addition to thorough informed consent disclosing all relevant risks and benefits of the procedure.

In an embodiment, a deoxyribonuclease enzyme protein disclosed herein, or a composition comprising such a therapeutic compound, may be made into a liquid formulation. In an embodiment, liquid formulations suitable for enteral or parenteral administration include, without limitation, solutions, syrups, elixirs, dispersions, emulsions, and suspensions. In an embodiment, a therapeutic compound or composition disclosed herein intended for such administration may be prepared, without limitation, according to any method known to the art for the manufacture of pharmaceutical compositions. In an embodiment, in such liquid dosage forms, a therapeutic compound or composition disclosed herein may be admixed with, without limitation, (a) suitable aqueous and nonaqueous carriers, (b) diluents, (c) solvents, such as, without limitation, water, ethanol, propylene glycol, polyethyleneglycol, glycerol, vegetable oils, such as, without limitation, rapeseed oil and olive oil, and injectable organic esters such as ethyl oleate; and/or fluidity agents, such as, without limitation, surfactants or coating agents like lecithin. In the case of dispersions and suspensions, fluidity can also be controlled by maintaining a particular particle size. In an embodiment, in liquid formulations, a therapeutically effective amount of a therapeutic compound disclosed herein typically may be between about 0.0001% (w/v) to about 90% (w/v), 0.0001% (w/v) to about 80% (w/v), 0.0001% (w/v) to about 70% (w/v), 0.0001% (w/v) to about 60% (w/v), 0.0001% (w/v) to about 50% (w/v), 0.0001% (w/v) to about 40% (w/v), 0.0001% (w/v) to about 30% (w/v), 0.0001% (w/v) to about 20% (w/v), 0.0001% (w/v) to about 10% (w/v), about 0.001% (w/v) to about 90.0% (w/v), 0.001% (w/v) to about 80.0% (w/v), 0.001% (w/v) to about 70.0% (w/v), 0.001% (w/v) to about 60.0% (w/v), 0.001% (w/v) to about 0.0% (w/v), 0.001% (w/v) to about 40.0% (w/v), 0.001% (w/v) to about 30.0% (w/v), 0.001% (w/v) to about 20.0% (w/v), 0.001% (w/v) to about 10.0% (w/v) or about 0.01% (w/v) to about 90.0% (w/v), about 0.01% (w/v) to about 80.0% (w/v), about 0.01% (w/v) to about 70.0% (w/v), about 0.01% (w/v) to about 60.0% (w/v), about 0.01% (w/v) to about 50.0% (w/v), about 0.01% (w/v) to about 40.0% (w/v), about 0.01% (w/v) to about 30.0% (w/v)about 0.01% (w/v) to about 20.0% (w/v) or about 0.01% (w/v) to about 10.0% (w/v).

In an embodiment, liquid suspensions may be formulated, without limitation, by suspending a therapeutic compound, including, but not limited to a deoxyribonuclease enzyme protein disclosed herein in admixture with excipients suitable for the manufacture of aqueous suspensions. In an embodiment, such excipients are suspending agents, for example, without limitation, sodium carboxymethylcellulose, methylcellulose, hydroxypropylmethylcellulose, sodium alginate, pectin, polyvinyl pyrrolidone, polyvinyl alcohol, natural gum, agar, gum tragacanth and gum acacia; dispersing or wetting agents may be a naturally occurring phosphatide, for example lecithin, or condensation products of an alkylene oxide with fatty acids, for example, without limitation, polyoxyethylene stearate, or condensation products of ethylene oxide with long-chain aliphatic alcohols, for example, without limitation, heptadecaethyleneoxycetanol, or condensation products of ethylene oxide with partial esters derived from fatty acids, for example, without limitation, polyoxyethylene sorbitan monooleate.

As an additional aspect, the invention includes kits which comprise a composition of the invention packaged in a manner which facilitates its use for administration to subjects. In one embodiment, such a kit includes a compound or composition described herein (e.g., a composition comprising a deoxyribonuclease enzyme protein and/or its derivatives, variants or analogs and at least one cell comprising a chimeric antigen receptor (CAR) or a T cell receptor (TCR)), packaged in a container such as a sealed bottle or vessel, with a label affixed to the container or included in the package that describes use of the compound or composition in practicing the method. In one embodiment, the kit contains a first container having a composition comprising a deoxyribonuclease enzyme protein and a second container having a physiologically acceptable reconstitution solution for the composition in the first container. In one aspect, the compound or composition is packaged in a unit dosage form. The kit may further include a device suitable for administering the composition according to a specific route of administration. Preferably, the kit contains a label that describes use of the therapeutic protein or peptide composition.

In certain embodiments, a DNase enzyme is formulated in a pharmaceutical composition with a pharmaceutically acceptable carrier or excipient.

The formulations used in the methods of the invention may conveniently be presented in unit dosage form and may be prepared by methods known in the art. The amount of active ingredients that can be combined with a carrier material to produce a single dosage form will vary depending upon the host being treated and the particular mode of administration. The amount of active ingredients that can be combined with a carrier material to produce a single dosage form will generally be that amount of the compound which produces a therapeutic effect.

In general, the formulations can be prepared with a liquid carrier, or a finely divided solid carrier, or both, and then, if necessary, shaping the product.

Pharmaceutical compositions suitable for parenteral administration may comprise one or more active ingredients (a DNase and, optionally, another compound [e.g., an anti-cancer compound]) in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms can be made by forming microencapsule matrices of one or more active ingredients in biodegradable polymers such as polylactide-polyglycolide. Depending on the ratio of active ingredient to polymer, and the nature of the particular polymer employed, the rate of active ingredient's release can be controlled. Examples of other biodegradable polymers include poly(orthoesters) and poly(anhydrides). Depot injectable formulations are also prepared by entrapping the active ingredients in liposomes or microemulsions which are compatible with body tissue.

Formulations for oral administration can be in the form of capsules, cachets, pills, tablets, powders, granules, or as a solution or a suspension in an aqueous or non-aqueous liquid (e.g., as a mouthwash, as a composition to be swallowed, or as an enema), or as an oil-in-water or water-in-oil liquid emulsion, and the like, each containing a predetermined amount of one or more active ingredients.

In solid dosage forms for oral administration (capsules, tablets, pills, dragees, powders, granules, and the like), one or more active ingredients can be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Suspensions, in addition to one or more active ingredients, can contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Powders and sprays can contain, in addition to one or more active ingredients, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane

### Cancer and Other Treatments

In one aspect, the invention provides a method of treating a cancer in a subject in need thereof, comprising administering to the subject a deoxyribonuclease enzyme and at least one cell comprising a chimeric antigen receptor (CAR) or a T cell receptor (TCR).

In a related aspect, the invention provides a method of treating a cancer in a subject in need thereof, comprising administering to the subject a deoxyribonuclease enzyme and at least one cell comprising a chimeric antigen receptor (CAR) or a T cell receptor (TCR), wherein said deoxyribonuclease enzyme is effective to reduce expression of immunosuppressive proteins in tumor tissue and/or increase content of cells comprising a chimeric antigen receptor or T cell receptor within tumor tissue.

The methods of the invention can be used in subjects suffering from a broad range of cancers. Non-limiting examples of relevant cancers include, e.g., breast cancer, prostate cancer, multiple myeloma, transitional cell carcinoma, lung cancer (e.g., non-small cell lung cancer (NSCLC)), renal cancer, thyroid cancer, leukemia (e.g., chronic myeloid leukemia, acute myeloid leukemia, chronic lymphocytic leukemia, acute lymphocytic leukemia), lymphoma (e.g., B cell lymphoma, T cell lymphoma, non-Hodgkins lymphoma, Hodgkins lymphoma), head and neck cancer, esophageal cancer, stomach cancer, colon cancer, intestinal cancer, colorectal cancer, rectal cancer, pancreatic cancer, liver cancer, cancer of the bile duct, cancer of the gall bladder, ovarian cancer, uterine endometrial cancer, vaginal cancer, cervical cancer, bladder cancer, neuroblastoma, sarcoma, osteosarcoma, malignant melanoma, squamous cell cancer, bone cancer, including both primary bone cancers (e.g., osteosarcoma, chondrosarcoma, Ewing's sarcoma, fibrosarcoma, malignant fibrous hi stiocytoma, adamantinoma, giant cell tumor, and chordoma) and secondary (metastatic) bone cancers, soft tissue sarcoma, basal cell carcinoma, angiosarcoma, hemangiosarcoma, myxosarcoma, liposarcoma, osteogenic sarcoma, angiosarcoma, endotheliosarcoma, lymphangiosarcoma, lymphangioendotheliosarcoma, synovioma, testicular cancer, uterine cancer, gastrointestinal cancer, mesothelioma, leiomyosarcoma, rhabdomyosarcoma, adenocarcinoma, sweat gland carcinoma, sebaceous gland carcinoma, papillary carcinoma, Waldenstroom' s macroglobulinemia, papillary adenocarcinomas, cystadenocarcinoma, bronchogenic carcinoma, choriocarcinoma, seminoma, embryonal carcinoma, Wilms' tumor, epithelial carcinoma, glioma, glioblastoma, astrocytoma, medulloblastoma, craniopharyngioma, ependymoma, pinealoma, hemangioblastoma, acoustic neuroma, oligodendroglioma, meningioma, retinoblastoma, medullary carcinoma, thymoma, sarcoma, etc.

In some embodiments of any of the methods of the invention, the subject is human.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1. Deoxyribonuclease Enzyme Increases Therapeutic Efficacy of Chimeric Antigen Receptor (CAR) Bearing T Cells

Six- to eight-week- old female NOD SCID (CB17-Prkdc^{scid}/NcrCrl) mice with an average weight of 16 to 20 g were used. Lymphomas were engrafted by inoculating 5×10⁶ Raji cells in 200 μ1 of 0.9% saline solution subcutaneously into the left side of mice. Once tumors had reached a palpable volume of at least 50 mm³, mice were randomly assigned to 4 experimental groups:

Group “Control”: single IV injection of 3×10⁶ untransduced human CD8 T cells on day 10 after tumor inoculation;

Group “DNase I”: ten daily IV injections of human recombinant DNase I enzyme (Kevelt AS; SEQ ID NO: 4) at 4 mg/kg dose starting on day 10 after tumor inoculation;

Group “CAR19”: single IV injection of 3×10⁶ human CD8 T cells transduced with lentiviral vector coding for CD19 CAR [FMC63 scFv/IgG4hinge/IgG4 CH2-CH3/CD28TM/OX-40/CD3z] (Stepanov A V, Markov O V, Chernikov I V, Gladkikh D V, Zhang H, Jones T, Sen'kova A V, Chernolovskaya E L, Zenkova M A, Kalinin R S, Rubtsova M P, Meleshko A N, Genkin D D, Belogurov A A Jr, Xie J, Gabibov A G, Lerner R A. Autocrine-based selection of ligands for personalized CAR-T therapy of lymphoma. Sci Adv. 2018 Nov 14;4(1 1): eaau4580. doi: 10.1126/sciadv. aau4580. PMfD: 30443597; PMCID: PMC6235538) on day 10 after tumor inoculation;

Group “CAR19 with DNase I”: single IV injection of 3×10⁶ untransduced human CD8 T cells transduced with lentiviral vector coding for CD19 CAR [FMC63 scFv/IgG4hinge/IgG4 CH2-CH3/CD28TM/OX-40/CD3z] on day 10 after tumor inoculation plus ten daily IV injections of human recombinant DNase I enzyme at 4 mg/kg dose starting on day 10 after tumor inoculation.

Tumor volume was measured with calipers and estimated using the ellipsoidal formula. The data are shown in FIG. 1. The data clearly show that IV injections of human recombinant DNase I enzyme provide statistically significant (p<0.05) efficacy boost for CI) 19 CART cells.

### Example 2. AAV Gene Transfer of Deoxyribonuclease Enzyme Increases Recruitment of Chimeric Antigen Receptor (CAR) Bearing T Cells Into Tumor Tissue and Reduces Local Immunosuppression

AAV-DNase I vector was produced as descried in Xia Y, He J, Zhang H, et al. AAV-mediated gene transfer of DNase I in the liver of mice with colorectal cancer reduces liver metastasis and restores local innate and adaptive immune response [published online ahead of print, 2020 Aug. 19] Mol Oncol. 2020;14(11):2920-2935. doi:10.1002/1878-0261.12787. Briefly, AAV-DNase I vector was produced using polyethylenimine triple plasmid transfections of rep/cap plasmid encoding the Anc80L65 capsid, pCLS-014, and adenovirus helper plasmids in HEK293 cells with further purification on an iodixanol gradient at scale. The plasmid pAAV-ApoEHCR enhancer-hAAT promoter-hDNase I (hyperactive)-WPRE Xinact (pCLS-014) specifically comprised a transgene expression cassette consisting of (a) apolipoprotein E-hepatic control region (APOE-HCR enhancer) and human alpha-1-antitrypsin (hAAT) promoter, (b) a Kozak sequence, (c) human hyperactive, actin resistant, DNase I-variant containing the natural signal sequence, (d) a woodchuck hepatitis virus posttranscriptional element (WPRE) with the X protein coding region inactivated by mutating the start codon. The purified AAV-DNase I vector was reformulated in PBS supplemented with 35 mM NaCl and 0.001% Pluronic F68, and the titer (genome copies [GC] per mL) was determined by quantitative PCR. The purified AAV vector was analyzed by SDS/PAGE, with three bands of 60, 72, and 90 kDa observed in a ratio of ˜1:1:10, which corresponds to the VP1-3 proteins. AAV-null contained all the components of CLS-014 transgene expression cassette, with the exception that it was devoid of DNase I cDNA.

A scFv of monoclonal antibody specific to CEA has been generated using proprietary sc fv phage display library. A scFv along with a CD8α hinge, CD28 transmembrane domain, plus CD3ζ and CD28 signaling domains were cloned into a pMS3 retroviral vector. Mouse CEA targeting CAR T cells has been generated as described in Wang L, Ma N, Okamoto S, et al. Efficient tumor regression by adoptively transferred CEA-specific CAR-T cells associated with symptoms of mild cytokine release syndrome. Oncoimmunology. 2016;5(9):e1211218. Published 2016 Jul. 25. doi:10.1080/2162402X.2016.1211218. Whole spleen cells (1.5×10⁷ cells/5 mL) from C57BL/6 (CD45.1) congeneric mice were stimulated with immobilized anti-CD3 (1 mg/mL; 145-2C11) and soluble anti-CD28 (1 mg/mL; 37.51) in one well of a six-well plate. One day after the stimulation (on day 1), 5×10⁵ cells were transduced with the viral vector by using the RetroNectin-bound virus infection method, wherein virus solutions were preloaded onto Retro-Nectin (Takara Bio)-coated wells of a 24-well plate containing 1 mL culture medium. On day 3, the cells were transferred to a 50 mL flask containing 10 mL culture medium for expansion. On day 5, the cells were harvested and used for experiments. Recombinant human IL-2 (Novartis) at 60 IU/mL was added during culturing.

To establish the liver metastasis models, the anesthetized C57BL/6 mice were placed in a supine position. After disinfecting the skin in the area of surgery, a median abdominal incision was performed followed by mobilization of the duodenum to identify the portal vein. One million CEA+ MC32a cells were injected into the portal vein using a 30-G needle. After removal of the needle, bleeding was stopped by gently pressing the puncture site with a cotton swab. After injection, the intestine was repositioned and the abdominal wall was closed with nonabsorbable sutures.

On day 5 after the tumor challenge the mice (7 animals per group) were randomly assigned into 4 groups:
Group “Control”: control tumor bearing mice;
Group “CAR19”: IV injection of 5×10⁶ CAR-T cells;
Group “AAV-DNase I”: IV injection of AAV-DNase I vector at 1.5×10¹² GC per mouse; Group “CAR19 with AAV-DNase I”: IV injection of 5×10⁶ CAR-T cells in combination with AAV-DNase I vector at 1.5×10¹² GC per mouse dose.

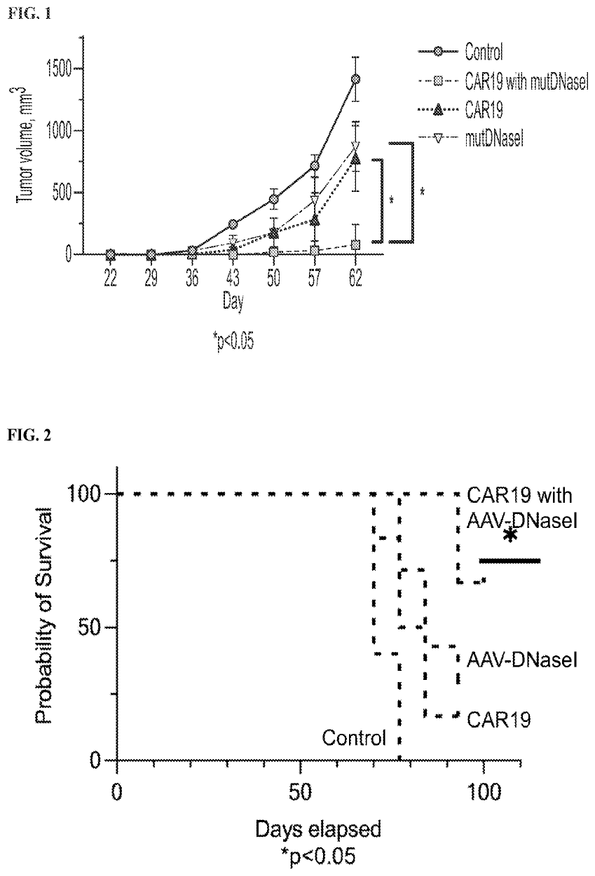

The survival graph is presented in FIG. 2. The data in FIG. 2 clearly shows meaningful survival (p<0.05) benefit from a combination of CAR T cells and AAV DNase I aene transfer.

At day 15, one animal from each of groups “control”, “CAR19”, and “CAR19 with AAV-DNase I” was euthanized (one mouse per group) and livers were harvested for examination. Metastatic tissue was analyzed by flow cytometry [as described in Xia Y, He J, Zhang H, et al. AAV-mediated gene transfer of DNase I in the liver of mice with colorectal cancer reduces liver metastasis and restores local innate and adaptive immune response. Mol Oncol. 2020;14(11):2920-2935. doi:10.1002/1878-0261.12787] for PD L1 expression using anti-PD-L1 antibody (clone 10F.9G2, Merck) and prevalence of CEA targeting CAR T cells using biotinylated protein L and streptavidin conjugated with FITC.

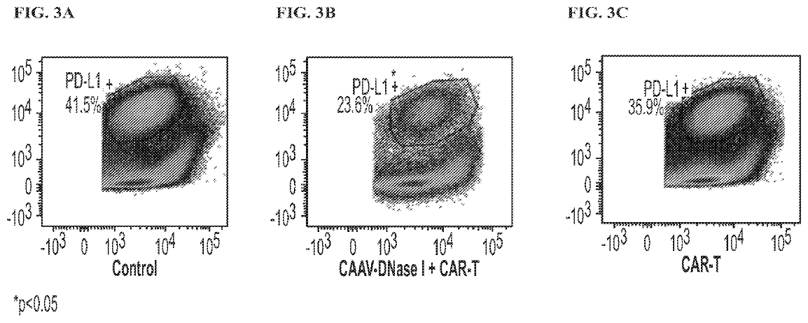

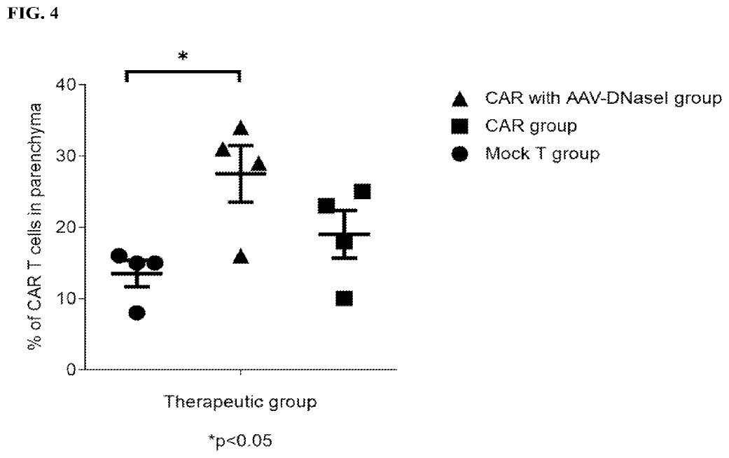

FIG. 3 shows percentage of metastatic MC32a cells expressing PD L1. Data show meaningful suppression of PD L1 expression (p<0.05) in metastatic MC32a cells from metastatic lesions from mice treated with combination of CAR T cells and AAV-DNase I gene transfer.

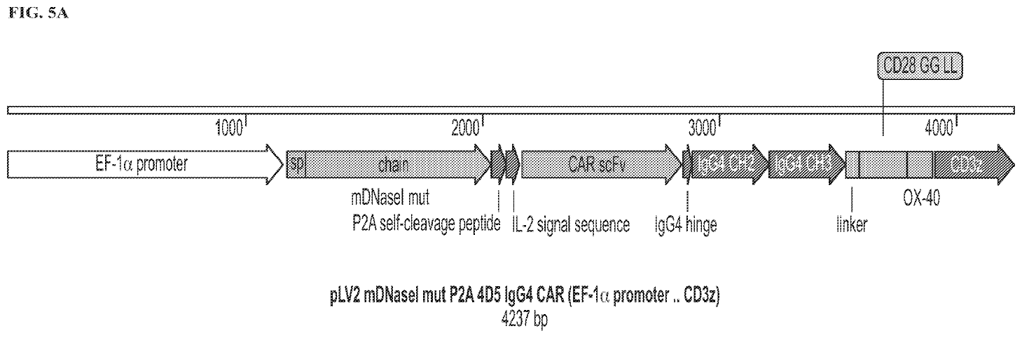

FIG. 4 shows percentage of CEA targeting CAR T cells in parenchyma of MC32a metastatic lesions. Data show meaningful increase (p<0.05) of number of CAR T cells in metastatic lesions from mice treated with combination of CAR T cells and AAV DNase I gene transfer.

### Example 3. Development of Chimeric Antigen Receptor (CAR) Bearing T Cells Expressing Deoxyribonuclease Enzyme

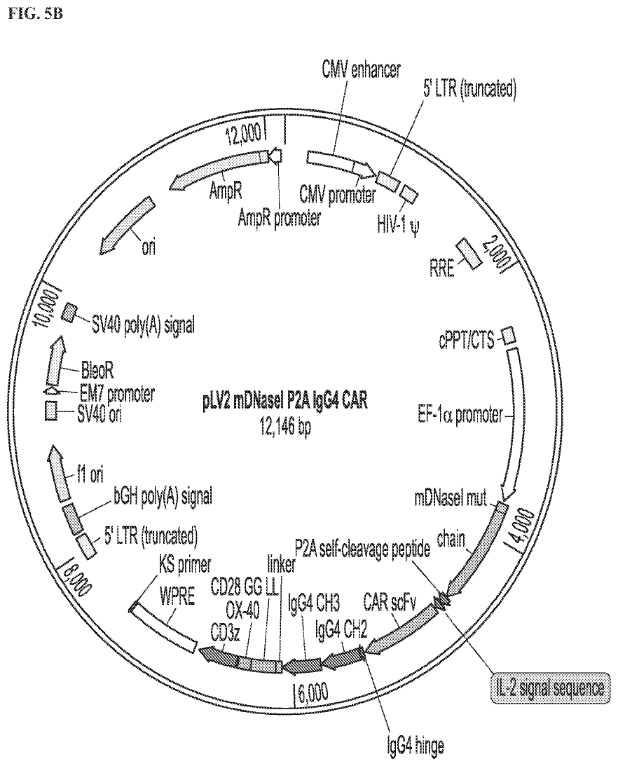

Human peripheral blood T cells from healthy donors were engineered with a FL1 CAR comprising CILDLPKFC/IgG1 Fc spacer domain (“CILDLPKFC” is disclosed as SEQ ID NO: 59), a GGGS linker (SEQ ID NO: 60), a CD28 transmembrane and intracellular region, and intracellular domains of the OX-40 and CD3zeta (Stepanov et al Ainocrine-based selection of ligands for personalized CAR-T therapy of lymphoma. Sci Adv. 2018, 4(11):eaau4580, doi.10.1126/sciadv.aau4580) with or without the gene of full-length human hyperactive actin resistant mutant DNase I (SEQ ID NO: 5) through self-cleavage peptide P2A. See FIG. 5.

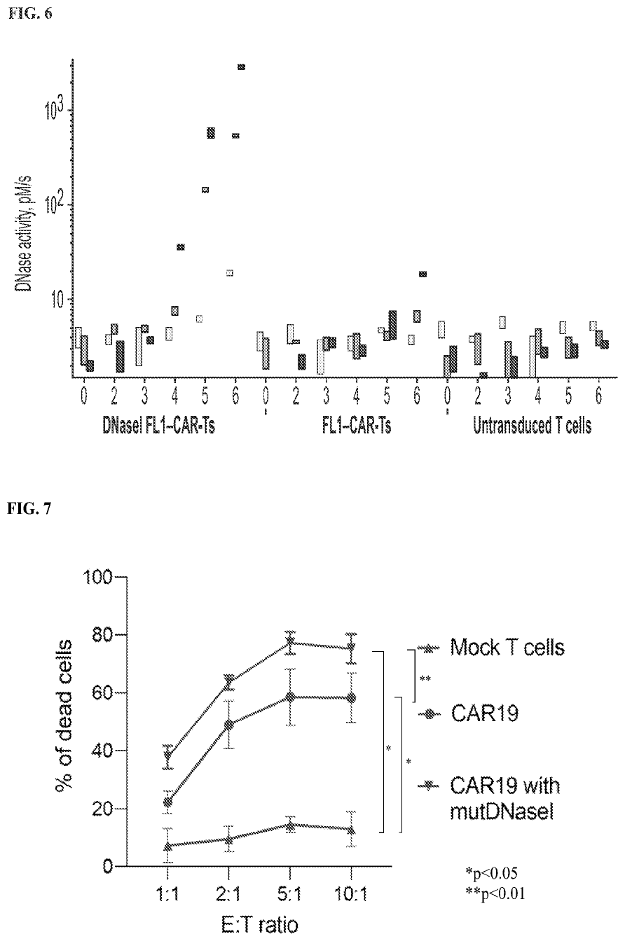

The DNase activity was analyzed using fluorescent probe (Terekhov et al., PNAS 2017, 114(10):2550-2555) in culture media of FL1-CAR-T cells, DNase I FL1-CAR-T cells and untransduced control T cells during 6-hour incubation of cells in complete RPMI 1640 media supplemented with human IL-2 (40 U/ml). All the experiments were performed in triplicate. Data are presented in FIG. 6. The data show gradual increase of DNase activity in culture media of DNase I FL1-CAR-T cells.

The cytotoxicity and specificity of engineered T cells were evaluated in a standard LDH release assay (CytoTox 96 Non-Radioactive Cytotoxicity Assay, Promega) following the manufacturer's recommendations. FL1-CAR-T cells, DNase I FL1-CAR-T cells and untransduced control T cells were co-incubated for 6 hours together with 10⁴ of the Raji-FL1 cells in complete RPMI 1640 media supplemented with human IL-2 (40 U/ml). All the experiments were performed in triplicate.

The data are shown in FIG. 7. The data clearly show that reprogramming of T cells to simultaneously express CAR and deoxyribonuclease enzyme provide superior cytotoxicity against target lymphoma cells (p<0.01).

### Example 4. Biological Activity of Chimeric Antigen Receptor (CAR) Bearing T Cells Expressing Deoxyribonuclease Enzyme

Six- to eight-week-old female NOD SCID (CB17-Prkdc^{scid}/NcrCrl) mice with an average weight of 16 to 20 g were used. Tumors were engrafted by inoculating 5×10⁶ Raji-FL1 CAR cells in 200 μl of 0.9% saline solution subcutaneously into the left side of mice. Once tumors had reached a palpable volume of at least 50 mm, mice were randomly assigned and treated as follows: At days 10 and 15, tumor-bearing mice were injected intravenously with 3×10⁶ a FL1 CART cells (Group 2; six mice), 3×10⁶ a FL1 CAR T cells expressing hyperactive actin resistant mutant DNase I (SEQ ID NO: 5) (Group 3; 6 mice) and placebo control (Group 1; 6 mice). Tumor volume was measured with calipers and estimated using the ellipsoidal formula. Animals were euthanized on day 20.

The data are shown in FIG. 8. The data clearly show that reprogramming of T cells to simultaneously express a CAR and a DNase enzyme provide superior efficacy for such dual reprogrammed I′ cells (p<0.05).

### Example 5. Treatment with DNase Increases Efficacy of CAR-T Cell Therapy

Human peripheral blood T cells from healthy donors were engineered with a CD19 CAR comprising FMC63 scFv/IgG4hinge/IgG4 CH2-CH3/CD28TM/OX-40/CD3z (as described in Stepanov et al., Autocrine-based selection of ligands for personalized CAR-T therapy of lymphoma. Sci Adv. 2018, 4(11):eaau4580. doi:10.1126/sciadv.aau4580). Six- to eight-week-old female NOD SCID (CB17-Prkdc^{skid}/NcrCrl) mice with an average weight of 16 to 20 g were used. Tumors were engrafted by inoculating 5×10⁶ Raji-CD19 CAR cells in 200 μl of 0.9% saline solution subcutaneously into the left side of mice. Once tumors had reached a palpable volume of at least 50 mm , mice were randomly assigned and treated as follows: at days 10 and 15, tumor-bearing mice were injected intravenously with CD19 chimeric antigen receptor (CAR) T cells at a dose of 0.5×10⁶ CAR-T cells/kg alone or in combinations with different DNase I regimens (human recombinant DNase I enzyme (Kevelt AS; SEQ ID NO: 4), administered by i.v. injection).

Animals were randomized to different groups:
Group 1 (n=5 mice)—treatment with CD19 CAR-T cells;
Group 2 (n=5 mice)—treatment with CD19 CAR-T cells+DNase I 500 μg/kg 1 time, 24 h before the initiation of CAR-T cell therapy;
Group 3 (n=5 mice)—treatment with CD19 CAR-T cells+DNase I 1000 μg/kg 2 times, 24 h before the initiation of CAR-T cell therapy and 24 h after;
Group 4 (n=5 mice)—treatment with CD19 CAR-T cells+DNase I 1000 μg/kg 2 times, 72 h before the initiation of CAR-T cell therapy and 30 days after;
Group 5 (n=5 mice)—treatment with CD19 CAR-T cells+DNase I 250 μg/kg, 3 days prior to the initiation of CAR-T cell therapy;
Group 6 (n=5 mice)—treatment with CD19 CAR-T cells+DNase I 2500 μg/kg, 1 day before and daily for 6 days starting from the initiation of CAR-T cell therapy;
Group 7 (n=5 mice)—treatment with CD19 CAR-T cells+DNase I 500 μg/kg 1 time, 7 days before the initiation of CAR-T cell therapy;
Group 8 (n=5 mice) - treatment with CD19 CAR-T cells+DNase I 500 μg/kg 1 time, 7 days before the initiation of CAR-T cell therapy and, after the initiation of CAR-T cell therapy, weekly for the next 8 weeks;
Group 9 (n=5 mice)—treatment with CD19 CAR-T cells+DNase I 1500 μg/kg 1 time, 28 days before the initiation of CAR-T cell therapy.

Clinical efficacy and side effects were monitored.

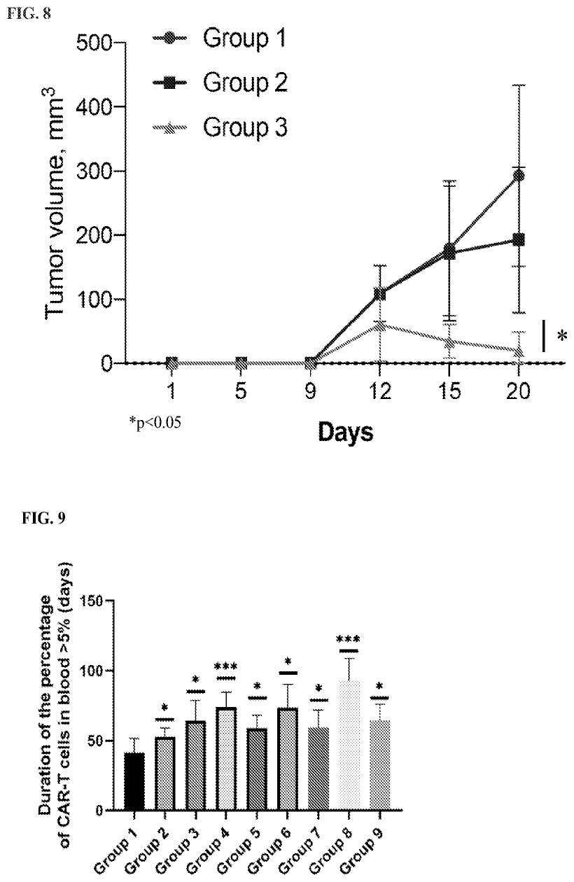

FIG. 9 shows flow cytometry detection of CAR-T cells in peripheral blood using biotinylated protein L and streptavidin conjugated with FITC (see Stepanov et al., Autocrine-based selection of ligands for personalized CAR-T therapy of lymphoma. Sci Adv. 2018, 4(11):eaau4580. doi:10.1126/sciadv.aau4580). The data demonstrate that the combined use of DNase and CAR-T cell therapy significantly (for all p<0.05) increases the persistence of CAR T cells in blood.

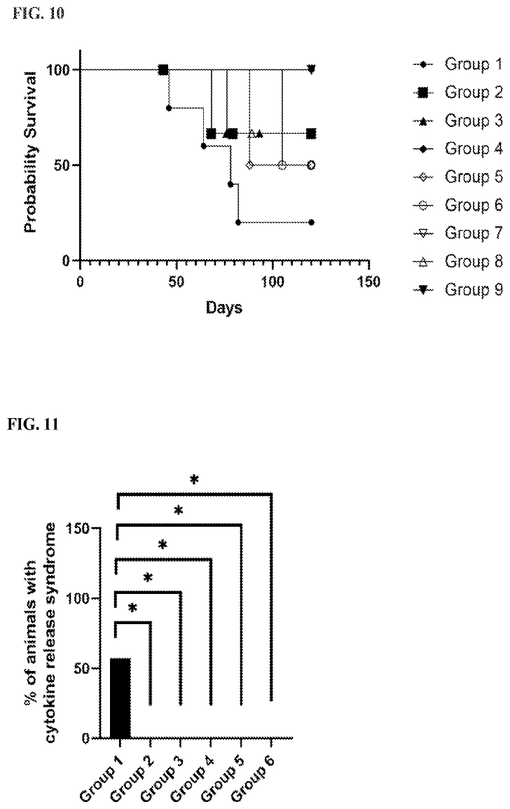

FIG. 10 demonstrates that the combined use of DNase and CAR-T cell therapy increases animal survival. All tested combinations of DNase I with CD19 CAR-T cells treatment show significant superiority to CD19 CAR-T cells treatment alone. Pretreatment of mice with high dose DNase I in group 9 produced particular advantage which might be linked to more extensive preconditioning of tumor microenvironment prior to CD19 CAR-T cells treatment.

### Example 6. Treatment with DNase Decreases Cytokine Release Syndrome Associated with CAR-T Cell Therapy

To model cytokine release syndrome (CRS) we used 6-8 week old female C.B.Igh-1b/GbmsTac-Prkdc^{scid}Lyst^{bg}N7 (SCID) mice (Charles River) were intraperitoneally injected with 3×10⁶ Raji cells. 3×10⁷ CD19 CAR-T cells (same as in Example 5) were injected intraperitoneally on the 25 th day after tumor growth. CAR-T cells were administered alone or in combinations with different DNase I regimens (human recombinant DNase I enzyme (Kevelt AS; SEQ ID NO: 4), administered by i.v. injection).

Animals were randomized to different groups:
Group 1 (n=5 mice)—treatment with CAR-T cells;
Group 2 (n=5 mice)—treatment with CAR-T cells+DNase I 500 μg/kg 1 time, 24 h before the initiation of CAR-T cell therapy;
Group 3 (n=5 mice)—treatment with CAR-T cells+DNase I 1000 μg/kg 24 h after the initiation of CAR-T cell therapy;
Group 4 (n=5 mice)—treatment with CAR-T cells+DNase I 1000 μg/kg 2 times, 72 h before the initiation of CAR-T cell therapy;
Group 5 (n=5 mice)—treatment with CAR-T cells+DNase I 500 μg/kg 1 time, 7 days before the initiation of CAR-T cell therapy;
Group 6 (n=5 mice)—treatment with CAR-T cells+DNase I 1500 μg/kg 1 time, 28 days before the initiation of CAR-T cell therapy.

The data on % of animals with CRS shown in FIG. 11 demonstrates that the use of DNase increases safety of CAR-T therapy and protects from CAR-T-related side effects.

Sequences

SEQ ID NO: 1-human DNase I, wild-type (WT), precursor; Genbank Accession No.
NP_005214.2; the secretory signal sequence is underlined:
MRGMKLLGALLALAALLQGAVSLKIAAFNIQTFGETKMSNATLVSYIVQILSRYDIA
LVQEVRDSHLTAVGKLLDNLNQDAPDTYHYVVSEPLGRNSYKERYLFVYRPDQVSA
VDSYYYDDGCEPCGNDTFNREPAIVRFFSRFTEVREFAIVPLHAAPGDAVAEIDALYD
VYLDVQEKWGLEDVMLMGDFNAGCSYVRPSQWSSIRLWTSPTFQWLIPDSADTTAT
PTHCAYDRIVVAGMLLRGAVVPDSALPFNFQAAYGLSDQLAQAISDHYPVEVMLK
SEQ ID NO: 2-human DNAse I mutant, precursor; the mutated residues as compared
to SEQ ID NO: 1 are in bold and underlined; the secretory signal sequence is
underlined:
MRGMKLLGALLALAALLQGAVSLKIAAFNIRTFGRTKMSNATLVSYIVQILSRYDIA
LVQEVRDSHLTAVGKLLDNLNQDAPDTYHYVVSEPLGRKSYKERYLFVYRPDQVS
AVDSYYYDDGCEPCGNDTFNREPFIVRFFSRFTEVREFAIVPLHAAPGDAVAEIDALY
DVYLDVQEKWGLEDVMLMGDFNAGCSYVRPSQWSSIRLWTSPTFQWLIPDSADTT
ATPTHCAYDRIVVAGMLLRGAVVPDSALPFNFQAAYGLSDQLAQAISDHYPVEVML
K
SEQ ID NO: 3-Anc80 VP1 capsid protein:
AADGYLPDWLEDNLSEGIREWDLKPGAPKPKANQQKQDDGRGLVLPGYKYLGPFN
GLDKGEPVNAADAAALEHDKAYDQQLKAGDNPYLRYNHADAEFQERLQEDTSFGG
NLGRAVFQAKKRVLEPLGLVEEGAKTAPGKKRPVEQSPQEPDSSSGIGKKGQQPAX¹
KRLNFGQTGDSESVPDPQPLGEPPAAPSGVGSNTMX²AGGGAPMADNNEGADGVGN
ASGNWHCDSTWLGDRVITTSTRTALPTYNNHLYKQISSQSGX³STNDNTYFGYSTPW
GYFDFNRFHCHFSPRDWQRLINNNWGFRPKX⁴LNFKLFNIQVKEVTTNDGTTTIANN
LTSTVQVFTDSEYQLPYVLGSAHQGCLPPFPADVFMIPQYGYLTLNNGSQAVGRSSF
YCLEYFPSQMLRTGNNFX⁵FSYTFEDVPFHSSYAHSQSLDRLNPLIDQYLYYLSRTQT
TSGTAGNRX⁶LQFSQAGPSSMANQAKNWLPGPCYRQQRVSKTX⁷NQNNNSNFAWTG
ATKYHLNGRDSLVNPGPAMATHKDDEDKFFPMSGVLIFGKQGAGNSNVDLDNVMI
TX⁸EEEIKTTNPVATEX⁹YGTVATNLQSX¹⁰NTAPATGTVNSQGALPGMVWQX¹¹RDV
YLQGPIWAKIPHTDGHFHPSPLMGGFGLKHPPPQILIKNTPVPANPPTTFSPAKFASFIT
QYSTGQVSVEIEWELQKENSKRWNPEIQYTSNYNKSTNVDFAVDINGVYSEPRPIGT
RYLTRNL
X¹ = K or R; X² = A or S; X³ = A or G; X⁴ = R or K; X⁵ = E or Q; X⁶ = T or E; X⁷ = A or T;
X⁸ = S or N; X⁹ = Q or E; X¹⁰ = S or A and X¹¹ = N or D.
SEQ ID NO: 4-mature wild-type (WT) human DNase I (without secretory signal
sequence; Genbank Accession No. 4AWN_A:
LKIAAFNIQTFGETKMSNATLVSYIVQILSRYDIALVQEVRDSHLTAVGKLLDNLNQD
APDTYHYVVSEPLGRNSYKERYLFVYRPDQVSAVDSYYYDDGCEPCGNDTFNREPA
IVRFFSRFTEVREFAIVPLHAAPGDAVAEIDALYDVYLDVQEKWGLEDVMLMGDFN
AGCSYVRPSQWSSIRLWTSPTFQWLIPDSADTTATPTHCAYDRIVVAGMLLRGAVVP
DSALPENFQAAYGLSDQLAQAISDHYPVEVMLK
SEQ ID NO: 5-mature human DNAse I mutant (without secretory signal sequence); the
mutated residues as compared to SEQ ID NO: 4 are in bold and underlined:
LKIAAFNIRTFGRTKMSNATLVSYIVQILSRYDIALVQEVRDSHLTAVGKLLDNLNQD
APDTYHYVVSEPLGRKSYKERYLFVYRPDQVSAVDSYYYDDGCEPCGNDTFNREPF
IVRFFSRFTEVREFAIVPLHAAPGDAVAEIDALYDVYLDVQEKWGLEDVMLMGDFN
AGCSYVRPSQWSSIRLWTSPTFQWLIPDSADTTATPTHCAYDRIVVAGMLLRGAVVP
DSALPFNFQAAYGLSDQLAQAISDHYPVEVMLK
SEQ ID NO: 6-secretory signal sequence of human DNase I:
MRGMKLLGALLALAALLQGAVS
SEQ ID NO: 7-secretory signal sequence of IL2:
MYRMQLLSCIALSLALVINS
SEQ ID NO: 8-human albumin promoter:
ACTAGTTCCAGATGGTAAATATACACAAGGGATTTAGTCAAACAATTTTTTGGCA
AGAATATTATGAATTTTGTAATCGGTTGGCAGCCAATGAAATACAAAGATGAGTC
TAGTTAATAATCTACAATTATTGGTTAAAGAAGTATATTAGTGCTAATTTCCCTCC
GTTTGTCCTAGCTTTTCTCTTCTGTCAACCCCACACGCCTTTGGCACC
SEQ ID NO: 9-Anc80 VP1 capsid protein:
AADGYLPDWLEDNLSEGIREWDLKPGAPKPKANQQKQDDGRGLVLPGYYLGPFNG
LDKGEPVNAADAAALEHDKAYDQQLKAGDNPYLRYNHADAEFQERLQEDTSFGGN
LGRAVFQAKKRVLEPLGLVEEGAKTAPGKKRPVEQSPQEPDSSSGIGKKGQQPAX¹K
RLNFGQTGDSESVPDPQPLGEPPAAPSGVGSNTMX²AGGGAPADNNEGADGVGNAS
GNWHCDSTWLGDRVITTSTRTALPTYNNHLYKQISSQSGX³STNDNTYFGYSTPWGY
FDFNRFHCHFSPRDWQRLINNNWGFRPKX⁴LNFKLFNIQVKEVTTNDGTTTIANNLTS
TVQVFTDSEYQLPYVLGSAHQGCLPPFPADVFMIPQYGYLTLNNGSQAVGRSSFYCL
EYFPSQMLRTGNNFX⁵FSYTFEDVPFHSSYAHSQSLDRLNPLIDQYLYYLSRTQTTSG
TAGNRX⁶LQFSQAGPSSANQAKNWLPGPCYRQQRVSKTX⁷NQNNNSNFAWTGATKY
HLNGRDSLVNPGPAMATHKDDEDKFFPMSGVLIFGKQGAGNSNVDLDNVITX⁸EEEI
KTTNPVATEX⁹YGTVATNLQSX¹⁰NTAPATGTVNSQGALPGVWQX¹¹RDVYLQGPIW
AKIPHTDGHFHPSPLMGGFGLKHPPPQILIKNTPVPANPPTTFSPAKFASFITQYSTGQ
VSVEIEELQKENSKRWNPEIQYTSNYNKSTNVDFAVDTNGVYSEPRPIGTRYLTRNL
X¹ = K or R; X² = A or S; X³ = A or G; X⁴ = R or K; X⁵ = E or Q; X⁶ = T or E;
X⁷ = A or T; X8 = S or N; X⁹ = Q or E; X¹⁰ = S or A and X¹¹ = N or D
SEQ ID NO: 10-human beta globin primer:
CAACTTCATCCACGTTCACC
SEQ ID NO: 11-forward NLRP3 primer:
GTTCTGAGCTCCAACCATTCT
SEQ ID NO: 12-reverse NLRP3 primer:
CACTGTGGGTCCTTCATCTTT
SEQ ID NO: 13-forward 16S universal bacterial RNA gene primer:
TCGTCGGCAGCGTCAGATGTGTATAAGAGACAGCCTACGGGNGGCWGCAG
SEQ ID NO: 14-reverse 16S universal bacterial RNA gene primer:
GTCTCGTGGGCTCGGAGATGTGTATAAGAGACAGGACTACHVGGGTATCTAATC
C
SEQ ID NO: 15-human anti-trypsin promoter sequence:
GATCTTGCTACCAGTGGAACAGCCACTAAGGATTCTGCAGTGAGAGCAGAGGGC
CAGCTAAGTGGTACTCTCCCAGAGACTGTCTGACTCACGCCACCCCCTCCACCTT
GGACACAGGACGCTGTGGTTTCTGAGCCAGGTACAATGACTCCTTTCGGTAAGTG
CAGTGGAAGCTGTACACTGCCCAGGCAAAGCGTCCGGGCAGCGTAGGCGGGCGA
CTCAGATCCCAGCCAGTGGACTTAGCCCCTGTTTGCTCCTCCGATAACTGGGGTG
ACCTTGGTTAATATTCACCAGCAGCCTCCCCCGTTGCCCCTCTGGATCCACTGCTT
AAATACGGACGAGGACAGGGCCCTGTCTCCTCAGCTTCAGGCACCACCACTGAC
CTGGGACAGTGAAT
SEQ ID NO: 16-Woodchuck hepatitis virus post-transcriptional regulatory element
that does not encode functional protein X:
AGTGGCGGCCGCTCGAGCTAGCGGCCGCTCTAGAAGATAATCAACCTCTGGATT
ACAAAATTTGTGAAAGATTGACTGGTATTCTTAACTATGTTGCTCCTTTTACGCTA
TGTGGATACGCTGCTTTAATGCCTTTGTATCATGCTATTGCTTCCCGTATGGCTTT
CATTTTCTCCTCCTTGTATAAATCCTGGTTGCTGTCTCTTTATGAGGAGTTGTGGC
CCGTTGTCAGGCAACGTGGCGTGGTGTGCACTGTGTTTGCTGACGCAACCCCCAC
TGGTTGGGGCATTGCCACCACCTGTCAGCTCCTTTCCGGGACTTTCGCTTTCCCCC
TCCCTATTGCCACGGCGGAACTCATCGCCGCCTGCCTTGCCCGCTGCTGGACAGG
GGCTCGGCTGTTGGGCACTGACAATTCCGTGGTGTTGTCGGGGAAATCATCGTCC
TTTCCTTGGCTGCTCGCCTGTGTTGCCACCTGGATTCTGCGCGGGACGTCCTTCTG
CTACGTCCCTTCGGCCCTCAATCCAGCGGACCTTCCTTCCCGCGGCCTGCTGCCG
GCTCTGCGGCCTCTTCCGCGTCTTCGCCTTCGCCCTCAGACGAGTCGGATCTCCCT
TTGGGCCGCCTCCCCGCATCGGACTAG
SEQ ID NO: 17-apolipoprotein E (ApoE) enhancer, hepatic control region (HCR):
AGGCTCAGAGGCACACAGGAGTTTCTGGGCTCACCCTGCCCCCTTCCAACCCCTC
AGTTCCCATCCTCCAGCAGCTGTTTGTGTGCTGCCTCTGAAGTCCACACTGAACA
AACTTCAGCCTACTCATGTCCCTAAAATGGGCAAACATTGCAAGCAGCAAACAG
CAAACACACAGCCCTCCCTGCCTGCTGACCTTGGAGCTGGGGCAGAGGTCAGAG
ACCTCTCTGGGCCCATGCCACCTCCAACATCCACTCGACCCCTTGGAATTTCGGT
GGAGAGGAGCAGAGGTTGTCCTGGCGTGGTTTAGGTAGTGTGAGAGG
SEQ ID NO: 18-a polynucleotide encoding human DNase I hyperactive variant of
SEQ ID NO: 5:
ATGAAGCTGCTGGGGGCGCTGCTGGCACTGGCGGCCCTACTGCAGGGGGCCGTG
TCCCTGAAGATCGCAGCCTTCAACATCAGGACATTTGGGAGGACCAAGATGTCC
AATGCCACCCTCGTCAGCTACATTGTGCAGATCCTGAGCCGCTATGACATCGCCC
TGGTCCAGGAGGTCAGAGACAGCCACCTGACTGCCGTGGGGAAGCTGCTGGACA
ACCTCAATCAGGATGCACCAGACACCTATCACTACGTGGTCAGTGAGCCACTGG
GACGGAAGAGCTATAAGGAGCGCTACCTGTTCGTGTACAGGCCTGACCAGGTGT
CTGCGGTGGACAGCTACTACTACGATGATGGCTGCGAGCCCTGCGGGAACGACA
CCTTCAACCGAGAGCCATTCATTGTCAGGTTCTTCTCCCGGTTCACAGAGGTCAG
GGAGTTTGCCATTGTTCCCCTGCATGCGGCCCCGGGGGACGCAGTAGCCGAGATC
GACGCTCTCTATGACGTCTACCTGGATGTCCAAGAGAAATGGGGCTTGGAGGAC
GTCATGTTGATGGGCGACTTCAATGCGGGCTGCAGCTATGTGAGACCCTCCCAGT
GGTCATCCATCCGCCTGTGGACAAGCCCCACCTTCCAGTGGCTGATCCCCGACAG
CGCTGACACCACAGCTACACCCACGCACTGTGCCTATGACAGGATCGTGGTTGCA
GGGATGCTGCTCCGAGGCGCCGTTGTTCCCGACTCGGCTCTTCCCTTTAACTTCCA
GGCTGCCTATGGCCTGAGTGACCAACTGGCCCAAGCCATCAGTGACCACTATCCA
GTGGAGGTGATGCTGAAGTGA
SEQ ID NO: 19-a polynucleotide encoding human DNAse I mutant precursor of SEQ
ID NO: 2 (secretory signal sequence underlined):
ATGAGGGGCATGAAGCTGCTGGGGGCGCTGCTGGCACTGGCGGCCCTACTGCAG
GGGGCCGTGTCCCTGAAGATCGCAGCCTTCAACATCAGGACATTTGGGAGGACC
AAGATGTCCAATGCCACCCTCGTCAGCTACATTGTGCAGATCCTGAGCCGCTATG
ACATCGCCCTGGTCCAGGAGGTCAGAGACAGCCACCTGACTGCCGTGGGGAAGC
TGCTGGACAACCTCAATCAGGATGCACCAGACACCTATCACTACGTGGTCAGTG
AGCCACTGGGACGGAAGAGCTATAAGGAGCGCTACCTGTTCGTGTACAGGCCTG
ACCAGGTGTCTGCGGTGGACAGCTACTACTACGATGATGGCTGCGAGCCCTGCG
GGAACGACACCTTCAACCGAGAGCCATTCATTGTCAGGTTCTTCTCCCGGTTCAC
AGAGGTCAGGGAGTTTGCCATTGTTCCCCTGCATGCGGCCCCGGGGGACGCAGT
AGCCGAGATCGACGCTCTCTATGACGTCTACCTGGATGTCCAAGAGAAATGGGG
CTTGGAGGACGTCATGTTGATGGGCGACTTCAATGCGGGCTGCAGCTATGTGAG
ACCCTCCCAGTGGTCATCCATCCGCCTGTGGACAAGCCCCACCTTCCAGTGGCTG
ATCCCCGACAGCGCTGACACCACAGCTACACCCACGCACTGTGCCTATGACAGG
ATCGTGGTTGCAGGGATGCTGCTCCGAGGCGCCGTTGTTCCCGACTCGGCTCTTC
CCTTTAACTTCCAGGCTGCCTATGGCCTGAGTGACCAACTGGCCCAAGCCATCAG
TGACCACTATCCAGTGGAGGTGATGCTGAAGTGA
SEQ ID NO: 20-a polynucleotide encoding the secretory signal sequence (SEQ ID NO:
6) of human DNase I:
ATGAGGGGCATGAAGCTGCTGGGGGCGCTGCTGGCACTGGCGGCCCTACTGCAG
GGGGCCGTGTCC
SEQ ID NO: 21-a polynucleotide encoding the mature human DNAse I mutant
(without secretory signal sequence) of SEQ ID NO: 5:
CTGAAGATCGCAGCCTTCAACATCAGGACATTTGGGAGGACCAAGATGTCCAAT
GCCACCCTCGTCAGCTACATTGTGCAGATCCTGAGCCGCTATGACATCGCCCTGG
TCCAGGAGGTCAGAGACAGCCACCTGACTGCCGTGGGGAAGCTGCTGGACAACC
TCAATCAGGATGCACCAGACACCTATCACTACGTGGTCAGTGAGCCACTGGGAC
GGAAGAGCTATAAGGAGCGCTACCTGTTCGTGTACAGGCCTGACCAGGTGTCTG
CGGTGGACAGCTACTACTACGATGATGGCTGCGAGCCCTGCGGGAACGACACCT
TCAACCGAGAGCCATTCATTGTCAGGTTCTTCTCCCGGTTCACAGAGGTCAGGGA
GTTTGCCATTGTTCCCCTGCATGCGGCCCCGGGGGACGCAGTAGCCGAGATCGAC
GCTCTCTATGACGTCTACCTGGATGTCCAAGAGAAATGGGGCTTGGAGGACGTC
ATGTTGATGGGCGACTTCAATGCGGGCTGCAGCTATGTGAGACCCTCCCAGTGGT
CATCCATCCGCCTGTGGACAAGCCCCACCTTCCAGTGGCTGATCCCCGACAGCGC
TGACACCACAGCTACACCCACGCACTGTGCCTATGACAGGATCGTGGTTGCAGG
GATGCTGCTCCGAGGCGCCGTTGTTCCCGACTCGGCTCTTCCCTTTAACTTCCAGG
CTGCCTATGGCCTGAGTGACCAACTGGCCCAAGCCATCAGTGACCACTATCCAGT
GGAGGTGATGCTGAAGTGA
SEQ ID NO: 22-a polynucleotide encoding human DNase I, wild-type (WT), precursor
of SEQ ID NO: 1:
ATGAGGGGCATGAAGCTGCTGGGGGCGCTGCTGGCACTGGCGGCCCTACTGCAG
GGGGCCGTGTCCCTGAAGATCGCAGCCTTCAACATCCAGACATTTGGGGAGACC
AAGATGTCCAATGCCACCCTCGTCAGCTACATTGTGCAGATCCTGAGCCGCTATG
ACATCGCCCTGGTCCAGGAGGTCAGAGACAGCCACCTGACTGCCGTGGGGAAGC
TGCTGGACAACCTCAATCAGGATGCACCAGACACCTATCACTACGTGGTCAGTG
AGCCACTGGGACGGAACAGCTATAAGGAGCGCTACCTGTTCGTGTACAGGCCTG
ACCAGGTGTCTGCGGTGGACAGCTACTACTACGATGATGGCTGCGAGCCCTGCG
GGAACGACACCTTCAACCGAGAGCCAGCCATTGTCAGGTTCTTCTCCCGGTTCAC
AGAGGTCAGGGAGTTTGCCATTGTTCCCCTGCATGCGGCCCCGGGGGACGCAGT
AGCCGAGATCGACGCTCTCTATGACGTCTACCTGGATGTCCAAGAGAAATGGGG
CTTGGAGGACGTCATGTTGATGGGCGACTTCAATGCGGGCTGCAGCTATGTGAG
ACCCTCCCAGTGGTCATCCATCCGCCTGTGGACAAGCCCCACCTTCCAGTGGCTG
ATCCCCGACAGCGCTGACACCACAGCTACACCCACGCACTGTGCCTATGACAGG
ATCGTGGTTGCAGGGATGCTGCTCCGAGGCGCCGTTGTTCCCGACTCGGCTCTTC
CCTTTAACTTCCAGGCTGCCTATGGCCTGAGTGACCAACTGGCCCAAGCCATCAG
TGACCACTATCCAGTGGAGGTGATGCTGAAG
SEQ ID NO: 23-a polynucleotide encoding the mature wild-type (WT) human DNase I
of SEQ ID NO: 4:
CTGAAGATCGCAGCCTTCAACATCCAGACATTTGGGGAGACCAAGATGTCCAAT
GCCACCCTCGTCAGCTACATTGTGCAGATCCTGAGCCGCTATGACATCGCCCTGG
TCCAGGAGGTCAGAGACAGCCACCTGACTGCCGTGGGGAAGCTGCTGGACAACC
TCAATCAGGATGCACCAGACACCTATCACTACGTGGTCAGTGAGCCACTGGGAC
GGAACAGCTATAAGGAGCGCTACCTGTTCGTGTACAGGCCTGACCAGGTGTCTG
CGGTGGACAGCTACTACTACGATGATGGCTGCGAGCCCTGCGGGAACGACACCT
TCAACCGAGAGCCAGCCATTGTCAGGTTCTTCTCCCGGTTCACAGAGGTCAGGGA
GTTTGCCATTGTTCCCCTGCATGCGGCCCCGGGGGACGCAGTAGCCGAGATCGAC
GCTCTCTATGACGTCTACCTGGATGTCCAAGAGAAATGGGGCTTGGAGGACGTC
ATGTTGATGGGCGACTTCAATGCGGGCTGCAGCTATGTGAGACCCTCCCAGTGGT
CATCCATCCGCCTGTGGACAAGCCCCACCTTCCAGTGGCTGATCCCCGACAGCGC
TGACACCACAGCTACACCCACGCACTGTGCCTATGACAGGATCGTGGTTGCAGG
GATGCTGCTCCGAGGCGCCGTTGTTCCCGACTCGGCTCTTCCCTTTAACTTCCAGG
CTGCCTATGGCCTGAGTGACCAACTGGCCCAAGCCATCAGTGACCACTATCCAGT
GGAGGTGATGCTGAAG
SEQ ID NO: 24-Mus musculus wild type DNase I, precursor; Genbank Accession No.
NP_034191.3; the secretory signal sequence is underlined:
MRYTGLMGTLLTLVNLLQLAGTLRIAAFNIRTFGETKMSNATLSVYFVKILSRYDIA
VIQEVRDSHLVAVGKLLDELNRDKPDTYRYVVSEPLGRKSYKEQYLFVYRPDQVSIL
DSYQYDDGCEPCGNDTFSREPAIVKFFSPYTEVQEFAIVPLHAAPTEAVSEIDALYDV
YLDVWQKWGLEDIMFMGDFNAGCSYVTSSQWSSIRLRTSPIFQWLIPDSADTTVTST
HCAYDRIVVAGALLQAAVVPNSAVPFDFQAEYGLSNQLAEAISDHYPVEVTLRKI
SEQ ID NO: 25-secretory signal sequence of Mus musculus wild type DNase I
MRYTGLMGTLLTLVNLLQLAGT
SEQ ID NO: 26-mature wild-type (WT) Mus musculus wild type DNase I
LRIAAFNIRTFGETKMSNATLSVYFVKILSRYDIAVIQEVRDSHLVAVGKLLDELNRD
KPDTYRYVVSEPLGRKSYKEQYLFVYRPDQVSILDSYQYDDGCEPCGNDTFSREPAI
VKFFSPYTEVQEFAIVPLHAAPTEAVSEIDALYDVYLDVWQKWGLEDIMFMGDFNA
GCSYVTSSQWSSIRLRTSPIFQWLIPDSADTTVTSTHCAYDRIVVAGALLQAAVVPNS
AVPFDFQAEYGLSNQLAEAISDHYPVEVTLRKI
SEQ ID NO: 27-a polynucleotide encoding the secretory signal sequence of Mus
musculus wild type DNase I
ATGCGGTACACAGGGCTAATGGGAACACTGCTCACCTTGGTCAACCTGCTGCAG
CTGGCTGGGACT
SEQ ID NO: 28-a polynucleotide encoding the mature wild-type (WT) Mus musculus
wild type DNase I
CTGAGAATTGCAGCCTTCAACATTCGGACTTTTGGGGAGACTAAGATGTCCAATG
CTACCCTCTCTGTATACTTTGTGAAAATCCTGAGTCGCTATGACATCGCTGTTATC
CAAGAGGTCAGAGACTCCCACCTGGTTGCTGTTGGGAAGCTCCTGGATGAACTC
AATCGGGACAAACCTGACACCTACCGCTATGTAGTCAGTGAGCCGCTGGGCCGC
AAAAGCTACAAGGAACAGTACCTTTTTGTGTACAGGCCTGACCAGGTGTCTATTC
TGGACAGCTATCAATATGATGATGGCTGTGAACCCTGTGGAAATGACACCTTCAG
CAGAGAGCCAGCCATTGTTAAGTTCTTTTCCCCATACACTGAGGTCCAAGAATTT
GCGATCGTGCCCTTGCATGCAGCCCCAACAGAAGCTGTGAGTGAGATCGACGCC
CTCTACGATGTTTACCTAGATGTCTGGCAAAAGTGGGGCCTGGAGGACATCATGT
TCATGGGAGACTTCAATGCTGGCTGCAGCTACGTCACTTCCTCCCAGTGGTCCTC
CATTCGCCTTCGGACAAGCCCCATCTTCCAGTGGCTGATCCCTGACAGTGCGGAC
ACCACAGTCACATCAACACACTGTGCTTATGACAGGATTGTGGTTGCTGGAGCTC
TGCTCCAGGCTGCTGTTGTTCCCAACTCGGCTGTTCCTTTTGATTTCCAAGCAGAA
TACGGACTTTCCAACCAGCTGGCTGAAGCCATCAGTGACCATTACCCAGTGGAG
GTGACACTCAGAAAAATCTGA
SEQ ID NO: 29-a polynucleotide encoding the Mus musculus wild type DNase I,
precursor
ATGCGGTACACAGGGCTAATGGGAACACTGCTCACCTTGGTCAACCTGCTGCAG
CTGGCTGGGACTCTGAGAATTGCAGCCTTCAACATTCGGACTTTTGGGGAGACTA
AGATGTCCAATGCTACCCTCTCTGTATACTTTGTGAAAATCCTGAGTCGCTATGA
CATCGCTGTTATCCAAGAGGTCAGAGACTCCCACCTGGTTGCTGTTGGGAAGCTC
CTGGATGAACTCAATCGGGACAAACCTGACACCTACCGCTATGTAGTCAGTGAG
CCGCTGGGCCGCAAAAGCTACAAGGAACAGTACCTTTTTGTGTACAGGCCTGAC
CAGGTGTCTATTCTGGACAGCTATCAATATGATGATGGCTGTGAACCCTGTGGAA
ATGACACCTTCAGCAGAGAGCCAGCCATTGTTAAGTTCTTTTCCCCATACACTGA
GGTCCAAGAATTTGCGATCGTGCCCTTGCATGCAGCCCCAACAGAAGCTGTGAGT
GAGATCGACGCCCTCTACGATGTTTACCTAGATGTCTGGCAAAAGTGGGGCCTGG
AGGACATCATGTTCATGGGAGACTTCAATGCTGGCTGCAGCTACGTCACTTCCTC
CCAGTGGTCCTCCATTCGCCTTCGGACAAGCCCCATCTTCCAGTGGCTGATCCCT
GACAGTGCGGACACCACAGTCACATCAACACACTGTGCTTATGACAGGATTGTG
GTTGCTGGAGCTCTGCTCCAGGCTGCTGTTGTTCCCAACTCGGCTGTTCCTTTTGA
TTTCCAAGCAGAATACGGACTTTCCAACCAGCTGGCTGAAGCCATCAGTGACCAT
TACCCAGTGGAGGTGACACTCAGAAAAATCTGA
SEQ ID NO: 30-Complete sequence of ApoEHCR enhancer-hAAT promoter-hDNaseI
(hyperactive)correct leader-WPRE Xinact (VR-18013AD)
AGGCTCAGAGGCACACAGGAGTTTCTGGGCTCACCCTGCCCCCTTCCAACCCCTC
AGTTCCCATCCTCCAGCAGCTGTTTGTGTGCTGCCTCTGAAGTCCACACTGAACA
AACTTCAGCCTACTCATGTCCCTAAAATGGGCAAACATTGCAAGCAGCAAACAG
CAAACACACAGCCCTCCCTGCCTGCTGACCTTGGAGCTGGGGCAGAGGTCAGAG
ACCTCTCTGGGCCCATGCCACCTCCAACATCCACTCGACCCCTTGGAATTTCGGT
GGAGAGGAGCAGAGGTTGTCCTGGCGTGGTTTAGGTAGTGTGAGAGGGATCTTG
CTACCAGTGGAACAGCCACTAAGGATTCTGCAGTGAGAGCAGAGGGCCAGCTAA
GTGGTACTCTCCCAGAGACTGTCTGACTCACGCCACCCCCTCCACCTTGGACACA
GGACGCTGTGGTTTCTGAGCCAGGTACAATGACTCCTTTCGGTAAGTGCAGTGGA
AGCTGTACACTGCCCAGGCAAAGCGTCCGGGCAGCGTAGGCGGGCGACTCAGAT
CCCAGCCAGTGGACTTAGCCCCTGTTTGCTCCTCCGATAACTGGGGTGACCTTGG
TTAATATTCACCAGCAGCCTCCCCCGTTGCCCCTCTGGATCCACTGCTTAAATAC
GGACGAGGACAGGGCCCTGTCTCCTCAGCTTCAGGCACCACCACTGACCTGGGA
CAGTGAATGCCGCCACCATGAGGGGCATGAAGCTGCTGGGGGCGCTGCTGGCAC
TGGCGGCCCTACTGCAGGGGGCCGTGTCCCTGAAGATCGCAGCCTTCAACATCA
GGACATTTGGGAGGACCAAGATGTCCAATGCCACCCTCGTCAGCTACATTGTGCA
GATCCTGAGCCGCTATGACATCGCCCTGGTCCAGGAGGTCAGAGACAGCCACCT
GACTGCCGTGGGGAAGCTGCTGGACAACCTCAATCAGGATGCACCAGACACCTA
TCACTACGTGGTCAGTGAGCCACTGGGACGGAAGAGCTATAAGGAGCGCTACCT
GTTCGTGTACAGGCCTGACCAGGTGTCTGCGGTGGACAGCTACTACTACGATGAT
GGCTGCGAGCCCTGCGGGAACGACACCTTCAACCGAGAGCCATTCATTGTCAGG
TTCTTCTCCCGGTTCACAGAGGTCAGGGAGTTTGCCATTGTTCCCCTGCATGCGG
CCCCGGGGGACGCAGTAGCCGAGATCGACGCTCTCTATGACGTCTACCTGGATGT
CCAAGAGAAATGGGGCTTGGAGGACGTCATGTTGATGGGCGACTTCAATGCGGG
CTGCAGCTATGTGAGACCCTCCCAGTGGTCATCCATCCGCCTGTGGACAAGCCCC
ACCTTCCAGTGGCTGATCCCCGACAGCGCTGACACCACAGCTACACCCACGCACT
GTGCCTATGACAGGATCGTGGTTGCAGGGATGCTGCTCCGAGGCGCCGTTGTTCC
CGACTCGGCTCTTCCCTTTAACTTCCAGGCTGCCTATGGCCTGAGTGACCAACTG
GCCCAAGCCATCAGTGACCACTATCCAGTGGAGGTGATGCTGAAGTGAAGTGGC
GGCCGCTCGAGCTAGCGGCCGCTCTAGAAGATAATCAACCTCTGGATTACAAAA
TTTGTGAAAGATTGACTGGTATTCTTAACTATGTTGCTCCTTTTACGCTATGTGGA
TACGCTGCTTTAATGCCTTTGTATCATGCTATTGCTTCCCGTATGGCTTTCATTTTC
TCCTCCTTGTATAAATCCTGGTTGCTGTCTCTTTATGAGGAGTTGTGGCCCGTTGT
CAGGCAACGTGGCGTGGTGTGCACTGTGTTTGCTGACGCAACCCCCACTGGTTGG
GGCATTGCCACCACCTGTCAGCTCCTTTCCGGGACTTTCGCTTTCCCCCTCCCTAT
TGCCACGGCGGAACTCATCGCCGCCTGCCTTGCCCGCTGCTGGACAGGGGCTCGG
CTGTTGGGCACTGACAATTCCGTGGTGTTGTCGGGGAAATCATCGTCCTTTCCTT
GGCTGCTCGCCTGTGTTGCCACCTGGATTCTGCGCGGGACGTCCTTCTGCTACGT
CCCTTCGGCCCTCAATCCAGCGGACCTTCCTTCCCGCGGCCTGCTGCCGGCTCTG
CGGCCTCTTCCGCGTCTTCGCCTTCGCCCTCAGACGAGTCGGATCTCCCTTTGGGC
CGCCTCCCCGCATCGGACTAG
(APOE HCR enhancer: bases 1-320; human alpha-1-antitrypsin promoter: bases 321-717;
Kozak sequence: bases 718-726; human DNasel hyperactive variant with natural full correct
leader sequence: bases 727-1575; WPRE X protein inactivated: bases 1576-2212)
SEQ ID NO: 31-Complete sequence of ApoEHCR enhancer-hAAT promoter-hDNaseI
wild type-WPRE Xinact (VR-18014AD)
AGGCTCAGAGGCACACAGGAGTTTCTGGGCTCACCCTGCCCCCTTCCAACCCCTC
AGTTCCCATCCTCCAGCAGCTGTTTGTGTGCTGCCTCTGAAGTCCACACTGAACA
AACTTCAGCCTACTCATGTCCCTAAAATGGGCAAACATTGCAAGCAGCAAACAG
CAAACACACAGCCCTCCCTGCCTGCTGACCTTGGAGCTGGGGCAGAGGTCAGAG
ACCTCTCTGGGCCCATGCCACCTCCAACATCCACTCGACCCCTTGGAATTTCGGT
GGAGAGGAGCAGAGGTTGTCCTGGCGTGGTTTAGGTAGTGTGAGAGGGATCTTG
CTACCAGTGGAACAGCCACTAAGGATTCTGCAGTGAGAGCAGAGGGCCAGCTAA
GTGGTACTCTCCCAGAGACTGTCTGACTCACGCCACCCCCTCCACCTTGGACACA
GGACGCTGTGGTTTCTGAGCCAGGTACAATGACTCCTTTCGGTAAGTGCAGTGGA
AGCTGTACACTGCCCAGGCAAAGCGTCCGGGCAGCGTAGGCGGGCGACTCAGAT
CCCAGCCAGTGGACTTAGCCCCTGTTTGCTCCTCCGATAACTGGGGTGACCTTGG
TTAATATTCACCAGCAGCCTCCCCCGTTGCCCCTCTGGATCCACTGCTTAAATAC
GGACGAGGACAGGGCCCTGTCTCCTCAGCTTCAGGCACCACCACTGACCTGGGA
CAGTGAATGCCGCCACCATGAGGGGCATGAAGCTGCTGGGGGCGCTGCTGGCAC
TGGCGGCCCTACTGCAGGGGGCCGTGTCCCTGAAGATCGCAGCCTTCAACATCCA
GACATTTGGGGAGACCAAGATGTCCAATGCCACCCTCGTCAGCTACATTGTGCAG
ATCCTGAGCCGCTATGACATCGCCCTGGTCCAGGAGGTCAGAGACAGCCACCTG
ACTGCCGTGGGGAAGCTGCTGGACAACCTCAATCAGGATGCACCAGACACCTAT
CACTACGTGGTCAGTGAGCCACTGGGACGGAACAGCTATAAGGAGCGCTACCTG
TTCGTGTACAGGCCTGACCAGGTGTCTGCGGTGGACAGCTACTACTACGATGATG
GCTGCGAGCCCTGCGGGAACGACACCTTCAACCGAGAGCCAGCCATTGTCAGGT
TCTTCTCCCGGTTCACAGAGGTCAGGGAGTTTGCCATTGTTCCCCTGCATGCGGC
CCCGGGGGACGCAGTAGCCGAGATCGACGCTCTCTATGACGTCTACCTGGATGTC
CAAGAGAAATGGGGCTTGGAGGACGTCATGTTGATGGGCGACTTCAATGCGGGC
TGCAGCTATGTGAGACCCTCCCAGTGGTCATCCATCCGCCTGTGGACAAGCCCCA
CCTTCCAGTGGCTGATCCCCGACAGCGCTGACACCACAGCTACACCCACGCACTG
TGCCTATGACAGGATCGTGGTTGCAGGGATGCTGCTCCGAGGCGCCGTTGTTCCC
GACTCGGCTCTTCCCTTTAACTTCCAGGCTGCCTATGGCCTGAGTGACCAACTGG
CCCAAGCCATCAGTGACCACTATCCAGTGGAGGTGATGCTGAAGTGAAGTGGCG
GCCGCTCGAGCTAGCGGCCGCTCTAGAAGATAATCAACCTCTGGATTACAAAATT
TGTGAAAGATTGACTGGTATTCTTAACTATGTTGCTCCTTTTACGCTATGTGGATA
CGCTGCTTTAATGCCTTTGTATCATGCTATTGCTTCCCGTATGGCTTTCATTTTCTC
CTCCTTGTATAAATCCTGGTTGCTGTCTCTTTATGAGGAGTTGTGGCCCGTTGTCA
GGCAACGTGGCGTGGTGTGCACTGTGTTTGCTGACGCAACCCCCACTGGTTGGGG
CATTGCCACCACCTGTCAGCTCCTTTCCGGGACTTTCGCTTTCCCCCTCCCTATTG
CCACGGCGGAACTCATCGCCGCCTGCCTTGCCCGCTGCTGGACAGGGGCTCGGCT
GTTGGGCACTGACAATTCCGTGGTGTTGTCGGGGAAATCATCGTCCTTTCCTTGG
CTGCTCGCCTGTGTTGCCACCTGGATTCTGCGCGGGACGTCCTTCTGCTACGTCCC
TTCGGCCCTCAATCCAGCGGACCTTCCTTCCCGCGGCCTGCTGCCGGCTCTGCGG
CCTCTTCCGCGTCTTCGCCTTCGCCCTCAGACGAGTCGGATCTCCCTTTGGGCCGC
CTCCCCGCATCGGACTAG
(APOE HCR enhancer: bases 1-320; human alpha-1-antitrypsin promoter: bases 321-717;
Kozak sequence: bases 718-726; human DNasel wild type with natural full correct leader
sequence: bases 727-1575; WPRE X protein inactivated: bases 1576-2212)
SEQ ID NO: 32-a polynucleotide encoding human DNase I, wild-type (WT), precursor
of SEQ ID NO: 1 with stop codon:
ATGAGGGGCATGAAGCTGCTGGGGGCGCTGCTGGCACTGGCGGCCCTACTGCAG
GGGGCCGTGTCCCTGAAGATCGCAGCCTTCAACATCCAGACATTTGGGGAGACC
AAGATGTCCAATGCCACCCTCGTCAGCTACATTGTGCAGATCCTGAGCCGCTATG
ACATCGCCCTGGTCCAGGAGGTCAGAGACAGCCACCTGACTGCCGTGGGGAAGC
TGCTGGACAACCTCAATCAGGATGCACCAGACACCTATCACTACGTGGTCAGTG
AGCCACTGGGACGGAACAGCTATAAGGAGCGCTACCTGTTCGTGTACAGGCCTG
ACCAGGTGTCTGCGGTGGACAGCTACTACTACGATGATGGCTGCGAGCCCTGCG
GGAACGACACCTTCAACCGAGAGCCAGCCATTGTCAGGTTCTTCTCCCGGTTCAC
AGAGGTCAGGGAGTTTGCCATTGTTCCCCTGCATGCGGCCCCGGGGGACGCAGT
AGCCGAGATCGACGCTCTCTATGACGTCTACCTGGATGTCCAAGAGAAATGGGG
CTTGGAGGACGTCATGTTGATGGGCGACTTCAATGCGGGCTGCAGCTATGTGAG
ACCCTCCCAGTGGTCATCCATCCGCCTGTGGACAAGCCCCACCTTCCAGTGGCTG
ATCCCCGACAGCGCTGACACCACAGCTACACCCACGCACTGTGCCTATGACAGG
ATCGTGGTTGCAGGGATGCTGCTCCGAGGCGCCGTTGTTCCCGACTCGGCTCTTC
CCTTTAACTTCCAGGCTGCCTATGGCCTGAGTGACCAACTGGCCCAAGCCATCAG
TGACCACTATCCAGTGGAGGTGATGCTGAAGTGA
SEQ ID NO: 33-Kozak sequence
5′-GCCGCCACC-3′
SEQ ID NO: 34-Anc80L65 VP1 capsid protein
MAADGYLPDWLEDNLSEGIREWWDLKPGAPKPKANQQKQDDGRGLVLPGYKYLG
PFNGLDKGEPVNAADAAALEHDKAYDQQLKAGDNPYLRYNHADAEFQERLQEDTS
FGGNLGRAVFQAKKRVLEPLGLVEEGAKTAPGKKRPVEQSPQEPDSSSGIGKKGQQP
ARKRLNFGQTGDSESVPDPQPLGEPPAAPSGVGSNTMAAGGGAPMADNNEGADGV
GNASGNWHCDSTWLGDRVITTSTRTWALPTYNNHLYKQISSQSGGSTNDNTYFGYS
TPWGYFDFNRFHCHFSPRDWQRLINNNWGFRPKKLNFKLFNIQVKEVTTNDGTTTIA
NNLTSTVQVFTDSEYQLPYVLGSAHQGCLPPFPADVFMIPQYGYLTLNNGSQAVGRS
SFYCLEYFPSQMLRTGNNFQFSYTFEDVPFHSSYAHSQSLDRLMNPLIDQYLYYLSRT
QTTSGTAGNRTLQFSQAGPSSMANQAKNWLPGPCYRQQRVSKTTNQNNNSNFAWT
GATKYHLNGRDSLVNPGPAMATHKDDEDKFFPMSGVLIFGKQGAGNSNVDLDNVM
ITNEEEIKTTNPVATEEYGTVATNLQSANTAPATGTVNSQGALPGMVWQDRDVYLQ
GPIWAKIPHTDGHFHPSPLMGGFGLKHPPPQILIKNTPVPANPPTTFSPAKFASFITQYS
TGQVSVEIEWELQKENSKRWNPEIQYTSNYNKSTNVDFAVDINGVYSEPRPIGTRYL
TRNL
SEQ ID NO: 35-variant Anc80L65 VP1 capsid protein
MAADGYLPDWLEDNLSEGIREWWDLKPGAPKPKANQQKQDDGRGLVLPGYYLGP
FNGLDKGEPVNAADAAALEHDKAYDQQLKAGDNPYLRYNHADAEFQERLQEDTSF
GGNLGRAVFQAKKRVLEPLGLVEEGAKTAPGKKRPVEQSPQEPDSSSGIGKKGQQP
ARKRLNFGQTGDSESVPDPQPLGEPPAAPSGVGSNTMAAGGGAPADNNEGADGVG
NASGNWHCDSTWLGDRVITTSTRTWALPTYNNHLYKQISSQSGGSTNDNTYFGYST
PWGYFDFNRFHCHFSPRDWQRLINNNWGFRPKKLNFKLFNIQVKEVTTNDGTTTIAN
NLTSTVQVFTDSEYQLPYVLGSAHQGCLPPFPADVFMIPQYGYLTLNNGSQAVGRSS
FYCLEYFPSQMLRTGNNFQFSYTFEDVPFHSSYAHSQSLDRLMNPLIDQYLYYLSRT
QTTSGTAGNRTLQFSQAGPSSANQAKNWLPGPCYRQQRVSKTTNQNNNSNFAWTG
ATKYHLNGRDSLVNPGPAMATHKDDEDKFFPMSGVLIFGKQGAGNSNVDLDNVITN
EEEIKTTNPVATEEYGTVATNLQSANTAPATGTVNSQGALPGVWQDRDVYLQGPIW
AKIPHTDGHFHPSPLMGGFGLKHPPPQILIKNTPVPANPPTTFSPAKFASFITQYSTGQ
VSVEIEELQKENSKRWNPEIQYTSNYNKSTNVDFAVDTNGVYSEPRPIGTRYLTRNL
SEQ ID NO: 36-human synapsin promoter
AGTGCAAGTGGGTTTTAGGACCAGGATGAGGCGGGGTGGGGGTGCCTACCTGAC
GACCGACCCCGACCCACTGGACAAGCACCCAACCCCCATTCCCCAAATTGCGCA
TCCCCTATCAGAGAGGGGGAGGGGAAACAGGATGCGGCGAGGCGCGTGCGCAC
TGCCAGCTTCAGCACCGCGGACAGTGCCTTCGCCCCCGCCTGGCGGCGCGCGCCA
CCGCCGCCTCAGCACTGAAGGCGCGCTGACGTCACTCGCCGGTCCCCCGCAAACT
CCCCTTCCCGGCCACCTTGGTCGCGTCCGCGCCGCCGCCGGCCCAGCCGGACCGC
ACCACGCGAGGCGCGAGATAGGGGGGCACGGGCGCGACCATCTGCGCTGCGGC
GCCGGCGACTCAGCGCTGCCTCAGTCTGCGGTGGGCAGCGGAGGAGTCGTGTCG
TGCCTGAGAGCGCAG
SEQ ID NO: 37-CMV promoter
GCGGCCGCTCTAGAGAGCTTGGCCCATTGCATACGTTGTATCCATATCATAATAT
GTACATTTATATTGGCTCATGTCCAACATTACCGCCATGTTGACATTGATTATTGA
CTAGTTATTAATAGTAATCAATTACGGGGTCATTAGTTCATAGCCCATATATGGA
GTTCCGCGTTACATAACTTACGGTAAATGGCCCGCCTGGCTGACCGCCCAACGAC
CCCCGCCCATTGACGTCAATAATGACGTATGTTCCCATAGTAACGCCAATAGGGA
CTTTCCATTGACGTCAATGGGTGGAGTATTTACGGTAAACTGCCCACTTGGCAGT
ACATCAAGTGTATCATATGCCAAGTACGCCCCCTATTGACGTCAATGACGGTAAA
TGGCCCGCCTGGCATTATGCCCAGTACATGACCTTATGGGACTTTCCTACTTGGC
AGTACATCTACGTATTAGTCATCGCTATTACCATGGTGATGCGGTTTTGGCAGTA
CATCAATGGGCGTGGATAGCGGTTTGACTCACGGGGATTTCCAAGTCTCCACCCC
ATTGACGTCAATGGGAGTTTGTTTTGGCACCAAAATCAACGGGACTTTCCAAAAT
GTCGTAACAACTCCGCCCCATTGACGCAAATGGGCGGTAGGCGTGTACGGTGGG
AGGTCTATATAAGCAGAGCTCGTTTAGTGAACCGTCAGATCGCCTGGAGACGCC
ATCCACGCTGTTTTGACCTCCATAGAAGACACCGGGACCGATCCAGCCTCCGGT
SEQ ID NO: 38-F4/80 promoter
CCTCTGTCTGTCTGTCTGTCTCTTTGGTGTGATGTATGTGGTGTGTGTGTATTCTAC
AAGGTTGACATGATGACAGAATTTAATTTTCTTAGCAGCAAGCTCATGGATCCTG
GTGATAAATGCAGCATGACTTTACTGAAAAGGCTTTGTGATCTTGAAGAGTGGAT
TGACTTCACTGTCGGCAGCACATGCAATCTCACTTGTTTGGTGTAATGAAAGAAG
AGAATGAGAGGTGGAAGGGGGATGGTAATGTTGAAAAAAAGAATGGTACAGAG
GAAACTGAGGTTGGAGAGAGATGGGGTAGATGGTAAGAGATGGAGAAAGAGGG
AAGGAAATGGAGAGAAAGACAGAGAGACAGAGAGAGACACACAGAGAGACAC
ACAGAGACAGAGAGGAAGGGAAAGGGAAAGAGAAAGGAAGAGGAAGAGGGGG
AGGGGAAGGGGAAGGGGAAGGGGAAGGGAGAGGGAGAAATGTGGACACTAGC
CAGATTTAAGGGAGAAATTAGGGGGTTGCCAGTCTGTCCACCTCTGATGGTGGC
AACTCAGCAGAAAGCTGCTGGGCTCAGTCTGGCTTTGTTGAGCAACCCTGACTCC
ACCCCTTTTCTTCCCCACAAAGCAAGCTTTTAAAGGGAAGGCTTTCTTCATTGAA
TGACTGCCACAGTACG
SEQ ID NO: 39-TMEM119 promoter
GGGGGTGAGCGAGGGCTGCTGGGACCATTGCAGGGAACAATGATAATCTAGGCT
TGGTTCCTACCCAGAGAGCACGCACTCATCCTTCATGCACTCCCCTGTTCCAAAC
CCTCACTGGCTCCGTACTGCCTCCGACCTTCCGAGACTTTAGCCTGGCTCCTGTCA
ACATCTCTGACCCTTACTACATGATCCTCTCTTTGGTCCATGCTCCAGCCTAATCT
AATTGCGGTGGCTTGTGCGTGGTGGCATTCCCAGCCACCATACCTTTACCCACGC
TGGTCCTTCCATGCGGAATGCCTTTCCAGGGCCTGCTTTGCCCGCTTCTGCTCATA
CACAGGCATGCCCTCCAGGATGGCTTCCTACCTCTTTCCCTTGGGGGATTGATCT
CTCTGTCTTGGGGTTCTCGGAGCCCTTGACCTGACCCCTTTCTGTTTGGCAAAAAA
GTAATTTACCTCGGTGTCCTTCTCCCTGGTAGTCTGTGAGCTCCCCAAGGCTGGG
CTGTGCCTGATTCACCTCTGGAACTTGCTTAGCACAGTGCGTGGCCTGCTGCAGG
TGTTCATTGAGCACTTGCCGAATGAATGCATGAATGAATGAATGAATGAATGAAT
GCAAGGGGCTGCTAATCCACAGGACTCCTCAGGTCAGCCAGACGTCCCGGTTCC
AAGGCCTGCCACTGACTCACCTCAGGACCCTGCTTGAACCATTAGAACTCACCCT
GCCTCACTTTCCCCCTCTGTGAAATGGGGCTCCAACTCCTATTCAAGCTACTATCA
TTTGGGGGCATTGTGAGGCCACAGATCCCAGAACATCAGAGTCAGAGGTAGCCC
AGAAAGCTTCCCACCCATCCCTACAAATGGGAAACTGAGGTCTGGAGAGGGAAG
GGCAGAGTTGGGCTCCCTGTCTCAGGCTCGGACCCACCATCAGGCCTGTCTCTAA
AACGAATCCCAGCTCCCACGCTGCACCCTGAGCCTGGAAGCCTGAGCCACACAA
GGACGGGGAATTTTCCTTCCCACTTCCAGAGGCCTCTGAACCTCCCTGAGCTTGT
CCCCTTTGGAGGGTATTGGGCAGCAGCGTGGGCAGAACCCCAGCTCACTGTCTG
GGGGAGCGCTGCAGGACAGCCTTGTCTGTCTGTCTCAGCCTGCCCTGGGGACCCG
AGGTCAGGGAGGAAGTGCCGCATCTGGTCTTCCCCAGAGCGAGAGTGTGAGCAA
GGGTGGGATTGCGTGTGGCCCGAGAGTAGCCCCTCCCCTCCCCCTGTCCCCACCC
CAAACCCTCTTAATGAAATCAAGCTGGCCCTGCGGCCCAGCCGGGGAGGGAGGA
AGGAGGAGGGACGGGAGGAGGGACGGGAGGAGGGAGGGCGGGCAGGCGCCAG
CCCAGAGCAGCCCCGGGCACCAGCACGGACTCTCTCTTCCAGCCCAGGTGCCCCC
CACTCTCGCTCCATTCGGCGGGAGCACCCAGTCCTGTACGCCAAGGAACTGGTGA
GTCCTGGGGTCCCCTCCT
SEQ ID NO: 40-MEF2 promoter
ACATGCAGGCACTGTGGCCCCGAAAAACTCTTCAGAGGTGAGCGGCTTTCAGGA
TGAGAGGGCCAACAATGGCTCATCTCCTAAAGACCTCGATAAACATAATCAGTA
GCCCCAGAAAACACAACAGCTGTCCCAAGCCTATCCCCTTTGCACCTATCTCAGA
AAAGGCAACATGCAGAGTGGTCAGGAGTTCAAGTCCTCAGGGCCCTACTCTGAA
GCTGTGACCCTGACAGGTCACTTTAGCGCTCAGCTTCCCTTTCCTCATCTGTAAAA
TGGGAATAGAAACTGAATCTGCTTCATGGGGCCTTGTAAGTATTACATAAGCTGT
CATATGCAAAATGTCCATCACATAGTTCACCGCCCCAGGCATCCTAGAAACTATT
AAGTCACGATAAGGAGGTTGCTACTGTCCTGGGACCTGGGGAGCTCGGCTGTCC
AGTCCGAAAGGGCCTTCTCGCCCTCGAGACCCCTTCAAGTCCCTTTCTCTCCGGG
CCCGGGTCCCAGAGTCCCCTTGTGCTTGCTCCTGAGGGTGACCTGCCCGTGCCCC
GGGGCGAGCACAGGTGCGGCCCTGGCCACAATGCGGGCGTGGCGAGCCGACACC
CGGGGAGGCCAAGGGCGCCTCGAGGGAGGGAGGGAGGAGGCTGACCCGGGCGT
CCCGAAGGACCCGCCCGGCCCCGGACTCCGGGCGGCAGCCGGCCTCGCGCCCCG
CCCCCGCCCCGGGAGCCCCGCCCAGTCCCGTCGCGCCGCGCCGCCCCACCGCCCG
GGTTGGCTGCCCTGGAGGCCACGCGCGGCGATTTGCCACGCGCCGCGTCACCGG
GCCGCTCCCGGCCTGGGCTCCCGGGGGCTGGTCAGGGAGGTGGCGGCGGCTGAG
CGGCGGAGCGGGGGCGGCAGGGCGCGACGCCGCCGGGCCGCCCCGCCCTGGGA
GGCGCCCGGGCCCTCATCAAGTGACCAGTATCCCTTCCAGGGGAACACGGTCCTT
CAGAGGAAAGCGAGCTCCAACCCGCGGCCCCGGCGCCAAGCCGCCGTCATCTTC
TTCCTGTGCCAGGTGATCGTCTCCTCACCCACCCGGAAAAACATAGTCCGCCCCC
ACGTCCTTGTGGAATAGCGCCCGCTTCCAAGCACCGTGACTCTCTTTGCCCTACC
CTTGTCTTTCCCATTCCAATTACTCTAGAACCCACCGAGAGGATAATTCAGTCCT
GAAAAGAAACTGATGGGGAGAAGCGAGGAAGGGAACCCAGGAGGGAGGGGAG
GCAGGGCTGCGGAGGGACACCGAGGCGGCGGAGGTAGTGCGCACGCGCAGCAC
AGAACGAGTTCCGGTCTGGCCGAGGCTTGTCTCCTAAAAATAGCCCCGGTGTGG
GGATCCGTGCGCGGATGTCCCGGCGAGTCCCGGGCTGAAAGAGGCGGCTCCGGG
CGGCGCGAAGCGCTGGTGGCGGGCCCGGGCTGCGGCGTGTGCGCGCCCGCCAGC
TGCTCCGGAGATACGGTGAGGGCCGCGGGCAGCGGGGCTCAGTCCGCGAC
SEQ ID NO: 41-FoxP2 promoter
AGTGCAGCCTCAGGGGTTGAAGGTGAAATGTGAAATTTGCTGTAATTACTTACCC
AAAATGTCTGTATTAAAGGCTATTAGAAGCAGTTATTTTAAGTACTTCTTTATAC
ATCTTTCCCTCTTCTGTTTAGTATTTAAATGTGCAACAAAAGTAGTGGCAGATGTT
ACTCATCTGAAGAAACTAAAACAAGATAAGTCAGTGGCAGGAGTGGTGGCCGGT
TAAAGATCTCTTCGAAGTGCTTTGTTCAGTAACTGTTGTTTTGTCTCACATTAAGT
TGTAGGTGGGGTGCTGTTAAAGCTTGTCCACTCACGATGGTGTTCAGCTGAGACC
TGTGAGTTCAAGTTGTCTTGCCCAGAGTTAAAGGCCATAAAACTAAGACAGTGTC
GTTCCTGTCTTGGGTGTAACTCCTAAAAAACTAGTCAGTTCTTGCAATAGGAGGA
AAATTATTGCAAATAAACCTTTGATTTACCAGCTTCAGTAAGTTGTTCATTCTGTC
TCTGTAGCTGTAGTTTTTACCTTAACAGTTTGAGGCTACCATTTCTCTTCCTTTCAT
GCTTATGGGGTCCACACAAACCCTGCTAGGCTACCTACAGTATAAAATCTTAAAA
CTCCATGTGGTGTTGTGAGCTGGTAGCAGATGGACTTTCTTTTGCATCTGCTGTGT
ACAGACTTGGCTTTGTACTCTTTTCCTGGGGGAGAGAAAGTCTTTCTTCTCTGCTA
GTTGATTCCTGTTTTTGTGAAACAGCTAAGAAAACATCTGTGGCAGGCAGAAAAA
TTGACAAAGGATTGACAAACACCATTAGCTGAAAATTTTCCATTTCATTGCAACT
ATGCATTGTTTACTTTACTGTAAATATCTTAATACATCATCCTCTGAATATGCTGG
CAGAGAAGCTGGAGAACTGTGATTTCAATTAAGGTTAGTAATTGATGGTATCTAG
TGTTCAAAGGCTGAAGCTTGATTAACATCTGCTTGGACAAATTGTCATTGTGAAG
TGGTTTTGATGTGCATTGAAGATTATTTCTTTCTGGCTTATCTGATGTTGTGGCTG
TGAAATGCTTGTCTTGGGTTTGCTTATTTTTGTAAACTACTTCCTTTGGCTGTAAA
TTGCAGAGCACTGGAGCTTTACCAAAAGTGCAGTGTATAATGGTAAGCTTGTCCT
AATAAGGGAAGCAAGAAGTGTATTTATCACAGACATGAAAGCTAACCGAGGACT
TGAGAGACTCAAACTGGTGCTTTTGTCTCTCTCTCTCTGTCTTTCTCTCTCTCACA
CACACACTCACACACTCACACACATGCACACACACACATACACACACACAAAAA
TGAAGCACTTACTTTAGAAAGATTATGGTAAGCATGCTGGCTCAGTCTTGAACCT
TTGTCACCCCTCACGTTGCACACCAAAGACATACCCTAGTGATTAAATGCTGATT
TTGTGTACGATTGTCCACGGACGCCAAAACAATCACAGAGCTGCTTGATTTGTTT
TAATTACCAGCACAAAATGCCATCAGTCTGGGACGTGATCGGGCAGAGGTGT
SEQ ID NO: 42-hDNaseI(hyperactive)correct leader-WPRE.bGH
ATGAGGGGCATGAAGCTGCTGGGGGCGCTGCTGGCACTGGCGGCCCTACTGCAG
GGGGCCGTGTCCCTGAAGATCGCAGCCTTCAACATCAGGACATTTGGGAGGACC
AAGATGTCCAATGCCACCCTCGTCAGCTACATTGTGCAGATCCTGAGCCGCTATG
ACATCGCCCTGGTCCAGGAGGTCAGAGACAGCCACCTGACTGCCGTGGGGAAGC
TGCTGGACAACCTCAATCAGGATGCACCAGACACCTATCACTACGTGGTCAGTG
AGCCACTGGGACGGAAGAGCTATAAGGAGCGCTACCTGTTCGTGTACAGGCCTG
ACCAGGTGTCTGCGGTGGACAGCTACTACTACGATGATGGCTGCGAGCCCTGCG
GGAACGACACCTTCAACCGAGAGCCATTCATTGTCAGGTTCTTCTCCCGGTTCAC
AGAGGTCAGGGAGTTTGCCATTGTTCCCCTGCATGCGGCCCCGGGGGACGCAGT
AGCCGAGATCGACGCTCTCTATGACGTCTACCTGGATGTCCAAGAGAAATGGGG
CTTGGAGGACGTCATGTTGATGGGCGACTTCAATGCGGGCTGCAGCTATGTGAG
ACCCTCCCAGTGGTCATCCATCCGCCTGTGGACAAGCCCCACCTTCCAGTGGCTG
ATCCCCGACAGCGCTGACACCACAGCTACACCCACGCACTGTGCCTATGACAGG
ATCGTGGTTGCAGGGATGCTGCTCCGAGGCGCCGTTGTTCCCGACTCGGCTCTTC
CCTTTAACTTCCAGGCTGCCTATGGCCTGAGTGACCAACTGGCCCAAGCCATCAG
TGACCACTATCCAGTGGAGGTGATGCTGAAGTGAAGTGGCGGCCGCTCGAGCTA
GCGGCCGCTCTAGAAGATAATCAACCTCTGGATTACAAAATTTGTGAAAGATTG
ACTGGTATTCTTAACTATGTTGCTCCTTTTACGCTATGTGGATACGCTGCTTTAAT
GCCTTTGTATCATGCTATTGCTTCCCGTATGGCTTTCATTTTCTCCTCCTTGTATAA
ATCCTGGTTGCTGTCTCTTTATGAGGAGTTGTGGCCCGTTGTCAGGCAACGTGGC
GTGGTGTGCACTGTGTTTGCTGACGCAACCCCCACTGGTTGGGGCATTGCCACCA
CCTGTCAGCTCCTTTCCGGGACTTTCGCTTTCCCCCTCCCTATTGCCACGGCGGAA
CTCATCGCCGCCTGCCTTGCCCGCTGCTGGACAGGGGCTCGGCTGTTGGGCACTG
ACAATTCCGTGGTGTTGTCGGGGAAATCATCGTCCTTTCCTTGGCTGCTCGCCTGT
GTTGCCACCTGGATTCTGCGCGGGACGTCCTTCTGCTACGTCCCTTCGGCCCTCA
ATCCAGCGGACCTTCCTTCCCGCGGCCTGCTGCCGGCTCTGCGGCCTCTTCCGCG
TCTTCGCCTTCGCCCTCAGACGAGTCGGATCTCCCTTTGGGCCGCCTCCCCGCATC
GGACTAGAAGCTTGCCTCGAGCAGCGCTGCTCGAGAGATCTACGGGTGGCATCC
CTGTGACCCCTCCCCAGTGCCTCTCCTGGCCCTGGAAGTTGCCACTCCAGTGCCC
ACCAGCCTTGTCCTAATAAAATTAAGTTGCATCATTTTGTCTGACTAGGTGTCCTT
CTATAATATTATGGGGTGGAGGGGGGTGGTATGGAGCAAGGGGCAAGTTGGGAA
GACAACCTGTAGGGCCTGCGGGGTCTATTGGGAACCAAGCTGGAGTGCAGTGGC
ACAATCTTGGCTCACTGCAATCTCCGCCTCCTGGGTTCAAGCGATTCTCCTGCCTC
AGCCTCCCGAGTTGTTGGGATTCCAGGCATGCATGACCAGGCTCAGCTAATTTTT
GTTTTTTTGGTAGAGACGGGGTTTCACCATATTGGCCAGGCTGGTCTCCAACTCC
TAATCTCAGGTGATCTACCCACCTTGGCCTCCCAAATTGCTGGGATTACAGGCGT
GAACCACTGCTCCCTTCCCTGTCCTTCTGATTTTGTAGGTAACCACGTGCGGACC
GA
SEQ ID NO: 43-hDNaseI(wild type)WPRE.bGH
ATGAGGGGCATGAAGCTGCTGGGGGCGCTGCTGGCACTGGCGGCCCTACTGCAG
GGGGCCGTGTCCCTGAAGATCGCAGCCTTCAACATCCAGACATTTGGGGAGACC
AAGATGTCCAATGCCACCCTCGTCAGCTACATTGTGCAGATCCTGAGCCGCTATG
ACATCGCCCTGGTCCAGGAGGTCAGAGACAGCCACCTGACTGCCGTGGGGAAGC
TGCTGGACAACCTCAATCAGGATGCACCAGACACCTATCACTACGTGGTCAGTG
AGCCACTGGGACGGAACAGCTATAAGGAGCGCTACCTGTTCGTGTACAGGCCTG
ACCAGGTGTCTGCGGTGGACAGCTACTACTACGATGATGGCTGCGAGCCCTGCG
GGAACGACACCTTCAACCGAGAGCCAGCCATTGTCAGGTTCTTCTCCCGGTTCAC
AGAGGTCAGGGAGTTTGCCATTGTTCCCCTGCATGCGGCCCCGGGGGACGCAGT
AGCCGAGATCGACGCTCTCTATGACGTCTACCTGGATGTCCAAGAGAAATGGGG
CTTGGAGGACGTCATGTTGATGGGCGACTTCAATGCGGGCTGCAGCTATGTGAG
ACCCTCCCAGTGGTCATCCATCCGCCTGTGGACAAGCCCCACCTTCCAGTGGCTG
ATCCCCGACAGCGCTGACACCACAGCTACACCCACGCACTGTGCCTATGACAGG
ATCGTGGTTGCAGGGATGCTGCTCCGAGGCGCCGTTGTTCCCGACTCGGCTCTTC
CCTTTAACTTCCAGGCTGCCTATGGCCTGAGTGACCAACTGGCCCAAGCCATCAG
TGACCACTATCCAGTGGAGGTGATGCTGAAGTGAAGTGGCGGCCGCTCGAGCTA
GCGGCCGCTCTAGAAGATAATCAACCTCTGGATTACAAAATTTGTGAAAGATTG
ACTGGTATTCTTAACTATGTTGCTCCTTTTACGCTATGTGGATACGCTGCTTTAAT
GCCTTTGTATCATGCTATTGCTTCCCGTATGGCTTTCATTTTCTCCTCCTTGTATAA
ATCCTGGTTGCTGTCTCTTTATGAGGAGTTGTGGCCCGTTGTCAGGCAACGTGGC
GTGGTGTGCACTGTGTTTGCTGACGCAACCCCCACTGGTTGGGGCATTGCCACCA
CCTGTCAGCTCCTTTCCGGGACTTTCGCTTTCCCCCTCCCTATTGCCACGGCGGAA
CTCATCGCCGCCTGCCTTGCCCGCTGCTGGACAGGGGCTCGGCTGTTGGGCACTG
ACAATTCCGTGGTGTTGTCGGGGAAATCATCGTCCTTTCCTTGGCTGCTCGCCTGT
GTTGCCACCTGGATTCTGCGCGGGACGTCCTTCTGCTACGTCCCTTCGGCCCTCA
ATCCAGCGGACCTTCCTTCCCGCGGCCTGCTGCCGGCTCTGCGGCCTCTTCCGCG
TCTTCGCCTTCGCCCTCAGACGAGTCGGATCTCCCTTTGGGCCGCCTCCCCGCATC
GGACTAGAAGCTTGCCTCGAGCAGCGCTGCTCGAGAGATCTACGGGTGGCATCC
CTGTGACCCCTCCCCAGTGCCTCTCCTGGCCCTGGAAGTTGCCACTCCAGTGCCC
ACCAGCCTTGTCCTAATAAAATTAAGTTGCATCATTTTGTCTGACTAGGTGTCCTT
CTATAATATTATGGGGTGGAGGGGGGTGGTATGGAGCAAGGGGCAAGTTGGGAA
GACAACCTGTAGGGCCTGCGGGGTCTATTGGGAACCAAGCTGGAGTGCAGTGGC
ACAATCTTGGCTCACTGCAATCTCCGCCTCCTGGGTTCAAGCGATTCTCCTGCCTC
AGCCTCCCGAGTTGTTGGGATTCCAGGCATGCATGACCAGGCTCAGCTAATTTTT
GTTTTTTTGGTAGAGACGGGGTTTCACCATATTGGCCAGGCTGGTCTCCAACTCC
TAATCTCAGGTGATCTACCCACCTTGGCCTCCCAAATTGCTGGGATTACAGGCGT
GAACCACTGCTCCCTTCCCTGTCCTTCTGATTTTGTAGGTAACCACGTGCGGACC
GA
SEQ ID NO: 44-human DNaseI mutant
ATGAGGGGCATGAAGCTGCTGGGGGCGCTGCTGGCACTGGCGGCCCTACTGCAG
GGGGCCGTGTCCCTGAAGATCGCAGCCTTCAACATCAGGACATTTGGGAGGACC
AAGATGTCCAATGCCACCCTCGTCAGCTACATTGTGCAGATCCTGAGCCGCTATG
ACATCGCCCTGGTCCAGGAGGTCAGAGACAGCCACCTGACTGCCGTGGGGAAGC
TGCTGGACAACCTCAATCAGGATGCACCAGACACCTATCACTACGTGGTCAGTG
AGCCACTGGGACGGAAGAGCTATAAGGAGCGCTACCTGTTCGTGTACAGGCCTG
ACCAGGTGTCTGCGGTGGACAGCTACTACTACGATGATGGCTGCGAGCCCTGCG
GGAACGACACCTTCAACCGAGAGCCATTCATTGTCAGGTTCTTCTCCCGGTTCAC
AGAGGTCAGGGAGTTTGCCATTGTTCCCCTGCATGCGGCCCCGGGGGACGCAGT
AGCCGAGATCGACGCTCTCTATGACGTCTACCTGGATGTCCAAGAGAAATGGGG
CTTGGAGGACGTCATGTTGATGGGCGACTTCAATGCGGGCTGCAGCTATGTGAG
ACCCTCCCAGTGGTCATCCATCCGCCTGTGGACAAGCCCCACCTTCCAGTGGCTG
ATCCCCGACAGCGCTGACACCACAGCTACACCCACGCACTGTGCCTATGACAGG
ATCGTGGTTGCAGGGATGCTGCTCCGAGGCGCCGTTGTTCCCGACTCGGCTCTTC
CCTTTAACTTCCAGGCTGCCTATGGCCTGAGTGACCAACTGGCCCAAGCCATCAG
TGACCACTATCCAGTGGAGGTGATGCTGAAGTGA
SEQ ID NO: 45-AAV5 capsid sequence (VP1)
MSFVDHPPDWLEEVGEGLREFLGLEAGPPKPKPNQQHQDQARGLVLPGYNYLGPGN
GLDRGEPVNRADEVAREHDISYNEQLEAGDNPYLKYNHADAEFQEKLADDTSFGGN
LGKAVFQAKKRVLEPFGLVEEGAKTAPTGKRIDDHFPKRKKARTEEDSKPSTSSDAE
AGPSGSQQLQIPAQPASSLGADTMSAGGGGPLGDNNQGADGVGNASGDWHCDSTW
MGDRVVTKSTRTWVLPSYNNHQYREIKSGSVDGSNANAYFGYSTPWGYFDFNRFH
SHWSPRDWQRLINNYWGFRPRSLRVKIFNIQVKEVTVQDSTTTIANNLTSTVQVFTD
DDYQLPYVVGNGTEGCLPAFPPQVFTLPQYGYATLNRDNTENPTERSSFFCLEYFPS
KMLRTGNNFEFTYNFEEVPFHSSFAPSQNLFKLANPLVDQYLYRFVSTNNTGGVQFN
KNLAGRYANTYKNWFPGPMGRTQGWNLGSGVNRASVSAFATTNRMELEGASYQV
PPQPNGMTNNLQGSNTYALENTMIFNSQPANPGTTATYLEGNMLITSESETQPVNRV
AYNVGGQMATNNQSSTTAPATGTYNLQEIVPGSVWMERDVYLQGPIWAKIPETGA
HFHPSPAMGGFGLKHPPPMMLIKNTPVPGNITSFSDVPVSSFITQYSTGQVTVEMEWE
LKKENSKRWNPEIQYTNNYNDPQFVDFAPDSTGEYRTTRPIGTRYLTRPL
SEQ ID NO: 46-AAV9 capsid sequence (VP1)
MAADGYLPDWLEDNLSEGIREWWALKPGAPQPKANQQHQDNARGLVLPGYKYLG
PGNGLDKGEPVNAADAAALEHDKAYDQQLKAGDNPYLKYNHADAEFQERLKEDT
SFGGNLGRAVFQAKKRLLEPLGLVEEAAKTAPGKKRPVEQSPQEPDSSAGIGKSGAQ
PAKKRLNFGQTGDTESVPDPQPIGEPPAAPSGVGSLTMASGGGAPVADNNEGADGV
GSSSGNWHCDSQWLGDRVITTSTRTWALPTYNNHLYKQISNSTSGGSSNDNAYFGY
STPWGYFDFNRFHCHFSPRDWQRLINNNWGFRPKRLNFKLFNIQVKEVTDNNGVKTI
ANNLTSTVQVFTDSDYQLPYVLGSAHEGCLPPFPADVFMIPQYGYLTLNDGSQAVG
RSSFYCLEYFPSQMLRTGNNFQFSYEFENVPFHSSYAHSQSLDRLMNPLIDQYLYYLS
KTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMI
TNEEEIKTTNPVATESYGQVATNHQSAQAQAQTGWVQNQGILPGMVWQDRDVYLQ
GPIWAKIPHTDGNFHPSPLMGGFGMKHPPPQILIKNTPVPADPPTAFNKDKLNSFITQ
YSTGQVSVEIEWELQKENSKRWNPEIQYTSNYYKSNNVEFAVNTEGVYSEPRPIGTR
YLTRNL
SEQ ID NO: 47-AAVLK03 VP1 capsid protein:
MAADGYLPDWLEDNLSEGIREWWALQPGAPKPKANQQHQDNARGLVLPGYKYLG
PGNGLDKGEPVNAADAAALEHDKAYDQQLKAGDNPYLKYNHADAEFQERLKEDT
SFGGNLGRAVFQAKKRLLEPLGLVEEAAKTAPGKKRPVDQSPQEPDSSSGVGKSGK
QPARKRLNFGQTGDSESVPDPQPLGEPPAAPTSLGSNTMASGGGAPMADNNEGADG
VGNSSGNWHCDSQWLGDRVITTSTRTWALPTYNNHLYKQISSQSGASNDNHYFGYS
TPWGYFDFNRFHCHFSPRDWQRLINNNWGFRPKKLSFKLFNIQVKEVTQNDGTTTIA
NNLTSTVQVFTDSEYQLPYVLGSAHQGCLPPFPADVFMVPQYGYLTLNNGSQAVGR
SSFYCLEYFPSQMLRTGNNFQFSYTFEDVPFHSSYAHSQSLDRLMNPLIDQYLYYLNR
TQGTTSGTTNQSRLLFSQAGPQSMSLQARNWLPGPCYRQQRLSKTANDNNNSNFPW
TAASKYHLNGRDSLVNPGPAMASHKDDEEKFFPMHGNLIFGKEGTTASNAELDNV
MITDEEEIRTTNPVATEQYGTVANNLQSSNTAPTTRTVNDQGALPGMVWQDRDVYL
QGPIWAKIPHTDGHFHPSPLMGGFGLKHPPPQIMIKNTPVPANPPTTFSPAKFASFITQ
YSTGQVSVEIEWELQKENSKRWNPEIQYTSNYNKSVNVDFTVDTNGVYSEPRPIGTR
YLTRPL
SEQ ID NO: 48-a polynucleotide encoding AAV-LK03 VP1 capsid protein:
ATGGCTGCTGACGGTTATCTTCCAGATTGGCTCGAGGACAACCTTTCTGAAGGCA
TTCGAGAGTGGTGGGCGCTGCAACCTGGAGCCCCTAAACCCAAGGCAAATCAAC
AACATCAGGACAACGCTCGGGGTCTTGTGCTTCCGGGTTACAAATACCTCGGACC
CGGCAACGGACTCGACAAGGGGGAACCCGTCAACGCAGCGGACGCGGCAGCCC
TCGAGCACGACAAGGCCTACGACCAGCAGCTCAAGGCCGGTGACAACCCCTACC
TCAAGTACAACCACGCCGACGCCGAGTTCCAGGAGCGGCTCAAAGAAGATACGT
CTTTTGGGGGCAACCTCGGGCGAGCAGTCTTCCAGGCCAAAAAGAGGCTTCTTG
AACCTCTTGGTCTGGTTGAGGAAGCGGCTAAGACGGCTCCTGGAAAGAAGAGGC
CTGTAGATCAGTCTCCTCAGGAACCGGACTCATCATCTGGTGTTGGCAAATCGGG
CAAACAGCCTGCCAGAAAAAGACTAAATTTCGGTCAGACTGGCGACTCAGAGTC
AGTCCCAGACCCTCAACCTCTCGGAGAACCACCAGCAGCCCCCACAAGTTTGGG
ATCTAATACAATGGCTTCAGGCGGTGGCGCACCAATGGCAGACAATAACGAGGG
TGCCGATGGAGTGGGTAATTCCTCAGGAAATTGGCATTGCGATTCCCAATGGCTG
GGCGACAGAGTCATCACCACCAGCACCAGAACCTGGGCCCTGCCCACTTACAAC
AACCATCTCTACAAGCAAATCTCCAGCCAATCAGGAGCTTCAAACGACAACCAC
TACTTTGGCTACAGCACCCCTTGGGGGTATTTTGACTTTAACAGATTCCACTGCC
ACTTCTCACCACGTGACTGGCAGCGACTCATTAACAACAACTGGGGATTCCGGCC
CAAGAAACTCAGCTTCAAGCTCTTCAACATCCAAGTTAAAGAGGTCACGCAGAA
CGATGGCACGACGACTATTGCCAATAACCTTACCAGCACGGTTCAAGTGTTTACG
GACTCGGAGTATCAGCTCCCGTACGTGCTCGGGTCGGCGCACCAAGGCTGTCTCC
CGCCGTTTCCAGCGGACGTCTTCATGGTCCCTCAGTATGGATACCTCACCCTGAA
CAACGGAAGTCAAGCGGTGGGACGCTCATCCTTTTACTGCCTGGAGTACTTCCCT
TCGCAGATGCTAAGGACTGGAAATAACTTCCAATTCAGCTATACCTTCGAGGATG
TACCTTTTCACAGCAGCTACGCTCACAGCCAGAGTTTGGATCGCTTGATGAATCC
TCTTATTGATCAGTATCTGTACTACCTGAACAGAACGCAAGGAACAACCTCTGGA
ACAACCAACCAATCACGGCTGCTTTTTAGCCAGGCTGGGCCTCAGTCTATGTCTT
TGCAGGCCAGAAATTGGCTACCTGGGCCCTGCTACCGGCAACAGAGACTTTCAA
AGACTGCTAACGACAACAACAACAGTAACTTTCCTTGGACAGCGGCCAGCAAAT
ATCATCTCAATGGCCGCGACTCGCTGGTGAATCCAGGACCAGCTATGGCCAGTCA
CAAGGACGATGAAGAAAAATTTTTCCCTATGCACGGCAATCTAATATTTGGCAA
AGAAGGGACAACGGCAAGTAACGCAGAATTAGATAATGTAATGATTACGGATGA
AGAAGAGATTCGTACCACCAATCCTGTGGCAACAGAGCAGTATGGAACTGTGGC
AAATAACTTGCAGAGCTCAAATACAGCTCCCACGACTAGAACTGTCAATGATCA
GGGGGCCTTACCTGGCATGGTGTGGCAAGATCGTGACGTGTACCTTCAAGGACCT
ATCTGGGCAAAGATTCCTCACACGGATGGACACTTTCATCCTTCTCCTCTGATGG
GAGGCTTTGGACTGAAACATCCGCCTCCTCAAATCATGATCAAAAATACTCCGGT
ACCGGCAAATCCTCCGACGACTTTCAGCCCGGCCAAGTTTGCTTCATTTATCACT
CAGTACTCCACTGGACAGGTCAGCGTGGAAATTGAGTGGGAGCTACAGAAAGAA
AACAGCAAACGTTGGAATCCAGAGATTCAGTACACTTCCAACTACAACAAGTCT
GTTAATGTGGACTTTACTGTAGACACTAATGGTGTTTATAGTGAACCTCGCCCCA
TTGGCACCCGTTACCTTACCCGTCCCCTGTAA
SEQ ID NO: 49-AAV-KP1 VP1 capsid protein:
MAADGYLPDWLEDTLSEGIRQWWKLKPGPPPPKPAERHKDDSRGLVLPGYKYLGP
GNGLDKGEPVNAADAAALEHDKAYDQQLKAGDNPYLRYNHADAEFQERLQEDTSF
GGNLGRAVFQAKKRVLEPLGLVEEPVKTAPGKKRPVEHSPVEPDSSSGTGKAGQQP
ARKRLNFGQTGDTDSAADPQPLGEPPAAPSGLGTGTMAAGGGAPMADNNEGADGV
GNASGNWHCDSTWLGDRVITTSTRTWALPTYNNHLYKQISSASTGASNDNHYFGYS
TPWGYFDFNRFHCHFSPRDWQRLINNNWGFRPKRLNFKLFNIQVKEVTQNEGTKTIA
NNLTSTVQVFTDSEYQLPYVLGSAHQGCLPPFPADVFMIPQYGYLTLNNGSQAMGR
SSFYCLEYFPSQMLRTGNNFQFTYTFEDVPFHSSYAHSQSLDRLMNPLIDQYLYYLN
RTQGTTSGTTNQSRLLFSQAGPQSMSLQARNWLPGPCYRQQRLSKTANDNNNSNFP
WTAASKYHLNGRDSLVNPGPAMASHKDDEEKFFPMHGNLIFGKEGTTASNAELDN
VMITDEEEIRTTNPVATEQYGTVANNLQSSNTAPTTRTVNDQGALPGMVWQDRDVY
LQGPIWAKIPHTDGHFHPSPLMGGFGLKHPPPQILIKNTPVPADPPTAFNKDKLNSFIT
QYSTGQVSVEIEWELQKENSKRWNPEIQYTSNYYKSTSVDFAVNTEGVYSEPRPIGT
RYLTRNL
SEQ ID NO: 50-a polynucleotide encoding AAV-KP1 VP1 capsid protein:
ATGGCTGCCGATGGTTATCTTCCAGATTGGCTCGAGGACACTCTCTCTGAAGGAA
TAAGACAGTGGTGGAAGCTCAAACCTGGCCCACCACCACCAAAGCCCGCAGAGC
GGCATAAGGACGACAGCAGGGGTCTTGTGCTTCCGGGTTACAAATACCTCGGAC
CCGGCAACGGACTCGACAAGGGGGAGCCCGTCAACGCGGCGGACGCAGCGGCC
CTCGAGCACGACAAGGCCTACGACCAGCAGCTCAAAGCGGGTGACAATCCGTAC
CTGCGGTATAACCACGCCGACGCCGAGTTTCAGGAGCGTCTGCAAGAAGATACG
TCTTTTGGGGGCAACCTCGGGCGAGCAGTCTTCCAGGCCAAGAAGCGGGTTCTCG
AACCTCTCGGTCTGGTTGAGGAACCTGTTAAGACGGCTCCGGGAAAAAAGAGGC
CGGTAGAGCACTCTCCTGTGGAGCCAGACTCCTCCTCGGGAACCGGAAAGGCGG
GCCAGCAGCCTGCGAGGAAGCGACTCAACTTTGGTCAGACTGGAGACACCGACT
CCGCCGCTGACCCCCAGCCTCTCGGAGAACCACCAGCAGCCCCCTCTGGTCTGGG
AACTGGTACAATGGCTGCAGGCGGTGGCGCTCCAATGGCAGACAATAACGAAGG
CGCCGACGGAGTGGGTAATGCCTCGGGAAATTGGCATTGCGATTCCACATGGCT
GGGCGACAGAGTCATCACCACCAGCACCCGCACCTGGGCCTTGCCCACCTACAA
TAACCACCTCTACAAGCAAATCTCCAGTGCTTCAACGGGGGCCAGCAACGACAA
CCACTACTTCGGCTACAGCACCCCCTGGGGGTACTTTGACTTCAACCGCTTCCAC
TGCCACTTCTCACCACGTGACTGGCAAAGACTCATCAACAACAACTGGGGATTCC
GGCCCAAGAGACTCAACTTCAAGCTCTTCAACATCCAGGTCAAGGAGGTCACGC
AGAATGAAGGCACCAAGACCATCGCCAATAACCTTACCAGCACGGTTCAGGTGT
TTACTGACTCGGAGTACCAGCTGCCGTACGTTCTCGGCTCTGCCCACCAGGGCTG
CCTGCCTCCGTTCCCGGCGGACGTGTTCATGATTCCGCAGTACGGCTACCTAACG
CTCAACAATGGCAGCCAGGCGATGGGTCGCTCGTCCTTCTACTGCCTGGAGTACT
TTCCGTCGCAGATGCTGAGAACCGGCAACAACTTCCAGTTTACTTACACCTTCGA
GGACGTGCCTTTCCACAGCAGCTACGCTCACAGCCAGAGTTTGGATCGCTTGATG
AATCCTCTTATTGATCAGTATCTGTACTACCTGAACAGAACGCAAGGAACAACCT
CTGGAACAACCAACCAATCACGGCTGCTTTTTAGCCAGGCTGGGCCTCAGTCTAT
GTCTTTGCAGGCCAGAAATTGGCTACCTGGGCCCTGCTACCGGCAACAGAGACTT
TCAAAGACTGCTAACGACAACAACAACAGTAACTTTCCTTGGACAGCGGCCAGC
AAATATCATCTCAATGGCCGCGACTCGCTGGTGAATCCAGGACCAGCTATGGCC
AGTCACAAGGACGATGAAGAAAAATTTTTCCCTATGCACGGCAATCTAATATTTG
GCAAAGAAGGGACAACGGCAAGTAACGCAGAATTAGATAATGTAATGATTACG
GATGAAGAAGAGATTCGTACCACCAATCCTGTGGCAACAGAGCAGTATGGAACT
GTGGCAAATAACTTGCAGAGCTCAAATACAGCTCCCACGACTAGAACTGTCAAT
GATCAGGGGGCCTTACCTGGCATGGTGTGGCAAGATCGTGACGTGTACCTTCAA
GGACCTATCTGGGCAAAGATTCCTCACACGGATGGACACTTTCATCCTTCTCCTC
TGATGGGCGGCTTTGGCCTGAAACATCCTCCGCCTCAGATCCTGATCAAGAACAC
GCCTGTACCTGCGGATCCTCCAACGGCCTTCAACAAGGACAAGCTGAACTCTTTC
ATCACCCAGTATTCTACTGGCCAAGTCAGCGTGGAGATCGAGTGGGAGCTGCAG
AAGGAAAACAGCAAGCGCTGGAACCCCGAGATCCAGTACACCTCCAACTACTAC
AAATCTACAAGTGTGGACTTTGCTGTTAATACAGAAGGCGTGTACTCTGAACCCC
GCCCCATTGGCACCCGTTACCTCACCCGTAATCTGTAA
SEQ ID NO: 51-Human DNase1L3 (Q13609), precursor (signal peptide is underlined):
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYT
DVKHRWKAENFIFMGDFNAGCSYVPKKAWKNIRLRTDPRFVWLIGDQEDTTVKKS
TNCAYDRIVLRGQEIVSSVVPKSNSVFDFQKAYKLTEEEALDVSDHFPVEFKLQSSRA
FTNSKKSVTLRKKTKSKRS
SEQ ID NO: 52-a polynucleotide encoding Human DNase1L3 (Q13609), precursor:
ATGTCACGGGAGCTGGCCCCACTGCTGCTTCTCCTCCTCTCCATCCACAGCGCCCTGGCC
ATGAGGATCTGCTCCTTCAACGTCAGGTCCTTTGGGGAAAGCAAGCAGGAAGACAAGAAT
GCCATGGATGTCATTGTGAAGGTCATCAAACGCTGTGACATCATACTCGTGATGGAAATC
AAGGACAGCAACAACAGGATCTGCCCCATACTGATGGAGAAGCTGAACAGAAATTCAAG
GAGAGGCATAACGTACAACTATGTGATTAGCTCTCGGCTTGGAAGAAACACATATAAAGA
ACAATATGCCTTTCTCTACAAGGAAAAGCTGGTGTCTGTGAAGAGGAGTTATCACTACCA
TGACTATCAGGATGGAGACGCAGATGTGTTTTCCAGGGAGCCCTTTGTGGTCTGGTTCCAA
TCTCCCCACACTGCTGTCAAAGACTTCGTGATTATCCCCCTGCACACCACCCCAGAGACA
TCCGTTAAGGAGATCGATGAGTTGGTTGAGGTCTACACGGACGTGAAACACCGCTGGAAG
GCGGAGAATTTCATTTTCATGGGTGACTTCAATGCCGGCTGCAGCTACGTCCCCAAGAAG
GCCTGGAAGAACATCCGCTTGAGGACTGACCCCAGGTTTGTTTGGCTGATCGGGGACCAA
GAGGACACCACGGTGAAGAAGAGCACCAACTGTGCATATGACAGGATTGTGCTTAGAGG
ACAAGAAATCGTCAGTTCTGTTGTTCCCAAGTCAAACAGTGTTTTTGACTTCCAGAAAGCT
TACAAGCTGACTGAAGAGGAGGCCCTGGATGTCAGCGACCACTTTCCAGTTGAATTTAAA
CTACAGTCTTCAAGGGCCTTCACCAACAGCAAAAAATCTGTCACTCTAAGGAAGAAAACA
AAGAGCAAACGCTCC
SEQ ID NO: 53-mature DNase1L3:
MRICSFNVRSFGESKQEDKNAMDVIVKVIKRCDIILVMEIKDSNNRICPILMEKLNRNSRRGITY
NYVISSRLGRNTYKEQYAFLYKEKLVSVKRSYHYHDYQDGDADVFSREPFVVWFQSPHTAV
KDFVIIPLHTTPETSVKEIDELVEVYTDVKHRWKAENFIFMGDFNAGCSYVPKKAWKNIRLRT
DPRFVWLIGDQEDTTVKKSTNCAYDRIVLRGQEIVSSVVPKSNSVFDFQKAYKLTEEEALDVS
DHFPVEFKLQSSRAFTNSKKSVTLRKKTKSKRS
NGTSG (SEQ ID NO: 54)
SGTH (SEQ ID NO: 55)
SDTH (SEQ ID NO: 56)
GGTAN (SEQ ID NO: 57)
DGSGL (SEQ ID NO: 58)
CILDLPKFC (SEQ ID NO: 59), IgG1 Fc spacer domain
GGGS linker (SEQ ID NO: 60)

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method of treating a cancer in a subject in need thereof, comprising administering to the subject a deoxyribonuclease enzyme and at least one cell comprising a chimeric antigen receptor (CAR) or a T cell receptor (TCR).

2. The method of claim 1, wherein said deoxyribonuclease enzyme is effective to increase content of cells comprising a chimeric antigen receptor or T cell receptor within tumor tissue.

3. The method of claim 1, wherein the deoxyribonuclease enzyme is administered parenterally as deoxyribonuclease enzyme protein.

4. The method of claim 1, wherein the deoxyribonuclease enzyme is encoded by a gene therapy vector.

5. The method of claim 1, wherein the deoxyribonuclease enzyme is coexpressed by the cell comprising the chimeric antigen receptor (CAR) or the T cell receptor (TCR).

6. The method of claim 1, wherein the CAR expressing cell or the TCR expressing cell is administered directly to the site of the tumor.

7. The method of claim 16, wherein the CAR expressing cell or TCR expressing cell is single-target or multi-target.

8. The methods of claim 1, wherein the CAR comprises an antigen binding domain capable of specific binding to one or more antigens selected from (i) a tumor antigen selected from CD5,CD7,CD19, CD28, mesothelin, CD123, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, IL-11Ra, PSCA, PRSS21, VEGFR2, LewisY, CD24, PDGFR-beta, SSEA-4, CD20, Folate receptor alpha, FR-1, c-MET, EGFR/CD133, IL13Ra2, HER2, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Plysialic acid, PLAC1, GloboH, NY-BR-1, UPK2, HAVCR1, ADRB3, PANX3, GPR20, LY6K, OR51E2, TARP, VVT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, 0Y-TES1, LCK, AKAP-4, SSX2, RAGE-1, human telomerase reverse transcriptase, RU1, RU2, intestinal carboxyl esterase, and mut hsp70-2; (ii) an antigen associated with a solid tumor; (iii) a solid tumor associated antigen selected from mesothelin, EGFRvIII, GD2, CLDN6, Tn Ag, PSMA, CD97, TAG72, CD44v6, CEA, EPCAM, KIT, IL-13Ra2, leguman, CD171, PSCA, TARP, MAD-CT-1, Lewis Y, CD24, folate receptor alpha, folate receptor beta, ERBBs, MUC1, EGFR, NCAM, PDGFR-beta, MAD-CT-2, Fos-related antigen, SSEA-4, neutrophil elastase, CAIX, HPV E6 E7, ML-IAP, NA17, ALK, androgen receptor plsialic acid, TRP-2, CYP1B1, PLAC1, GloboH, NY-BR-1, sperm protein 17, HMWMAA, beta human chorionic gonadotropin, AFP, thyroglobulin, RAGE-1, MN-CA IX, human telomerase reverse transcriptase, intestinal carboxyl esterase, and mut hsp 70-2; (iv) a solid tumor associated antigen present in/on a mesothelioma, a lung cancer, a pancreatic cancer, an esophageal adenocarcinoma, an ovarian cancer, a breast cancer, a colorectal cancer, a bladder cancer, or any combination thereof; (v) a tumor antigen that is associated with a hematological cancer;
(vi) a tumor antigen present in a disease chosen from acute leukemias including B-cell acute lymphoid leukemia (“BALL”), T-cell acute lymphoid leukemia (“TALL”), and acute lymphoid leukemia (ALL); or one or more chronic leukemias including chronic myelogenous leukemia (CIVIL) and chronic lymphoid leukemia (CLL); and (vi) a tumor antigen present in a therapy resistant cancer.

9. The method of claim 1, wherein the deoxyribonuclease enzyme comprises an amino acid sequence having at least 90% sequence identity to human DNase I enzyme.

10. The method of claim 1, wherein the deoxyribonuclease enzyme comprises an amino acid sequence having at least 90% sequence identity to amino acids 21 to 305 of DNase1-like 3 (D1L3) enzyme.

11. The method of claim 1, wherein the gene therapy vector is a recombinant adeno-associated virus (rAAV) expression vector comprising (i) a capsid protein and (ii) a nucleic acid comprising a promoter operably linked to a nucleotide sequence encoding an enzyme which has a deoxyribonuclease (DNase) activity.

12. The method of claim 11, wherein the promoter is a liver-specific promoter or a promoter is specific for tumor originator tissue or metastasis target tissue.

13. (canceled)

14. The method of claim 1, wherein the deoxyribonuclease enzyme protein is injected intravenously for at least 14 days or at least 16 days following infusion of the CAR expressing cells or TCR expressing cells.

15. (canceled)

16. The method of claim 1, wherein the deoxyribonuclease enzyme protein is injected intravenously for at least 3 to at least 7 days prior, together or following infusion of the CAR expressing cells or TCR expressing cells.

17. The method of claim 1, wherein the deoxyribonuclease enzyme protein is injected intravenously for at least 3 to 7 days following infusion of the CAR expressing cells or TCR expressing cells.

18.-19. (canceled)

20. The method of claim 1, wherein the deoxyribonuclease enzyme protein is injected intravenously for at least 14 days at 250 μg/kg/day.

21. The method of claim 1, wherein the CAR expressing cell or TCR expressing cell is further modified to express an immune checkpoint inhibitor molecule.

22. (canceled)

23. The method of claim 1, wherein the gene therapy vector encoding deoxyribonuclease enzyme is injected intravenously and/or at the tumor site at least 3 to at least 14 days prior to infusion of the CAR expressing cells or TCR expressing cells.

24. (canceled)

25. The method of claim 1, wherein the gene therapy vector encoding deoxyribonuclease enzyme is injected intravenously and/or at the tumor site simultaneously with infusion of CAR expressing cells or TCR expressing cells.

26.-33. (canceled)


SEQUENCE LISTING (60 sequences)

SEQ ID NO: 1 - protein, 282 aa, Homo sapiens [sequence omitted]

SEQ ID NO: 2 - protein, 282 aa, Artificial Sequence
  Description of Artificial Sequence Synthetic polypeptide
MRGMKLLGALLALAALLQGAVSLKIAAFNIRTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 3 - protein, 732 aa, Artificial Sequence
  Description of Artificial Sequence Synthetic polypeptide
  MOD_RES (166)..(166): Lys or Arg
  MOD_RES (202)..(202): Ala or Ser
  MOD_RES (263)..(263): Ala or Gly
  MOD_RES (308)..(308): Arg or Lys
  MOD_RES (408)..(408): Glu or Gln
  MOD_RES (456)..(456): Thr or Glu
  MOD_RES (489)..(489): Ala or Thr
  MOD_RES (558)..(558): Ser or Asn
  MOD_RES (572)..(572): Gln or Glu
  MOD_RES (583)..(583): Ser or Ala
  MOD_RES (605)..(605): Asn or Asp
AADGYLPDWLEDNLSEGIREWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPAXKRLNFGQTGDSESVPDPQPLGEPPAAPSGVGSNTMXAGGGAPMADNNEGADGVGNASGNWHCDSTWLGDRVITTSTRTALPTYNNHLYKQISSQSGXSTNDNTYFGYSTPWGYFDFNRFHCHFSPRDWQRLINNNWGFRPKXLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFXFSYTFEDVPFHSSYAHSQSLDRLNPLIDQYLYYLSRTQTTSGTAGNRXLQFSQAGPSSMANQAKNWLPGPCYRQQRVSKTXNQNNNSNFAWTGATKYHLNGRDSLVNPGPAMATHKDDEDKFFPMSGVLIFGKQGAGNSNVDLDNVMITXEEEIKTTNPVATEXYGTVATNLQSXNTAPATGTVNSQGALPGMVWQXRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 4 - protein, 260 aa, Homo sapiens
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 5 - protein, 260 aa, Artificial Sequence
  Description of Artificial Sequence Synthetic polypeptide
LKIAAFNIRTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 6 - protein, 22 aa, Homo sapiens
MRGMKLLGALLALAALLQGAVS

SEQ ID NO: 7 - protein, 20 aa, Homo sapiens
MYRMQLLSCIALSLALVTNS

SEQ ID NO: 8 - DNA, 214 bp, Homo sapiens
actagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcacc

SEQ ID NO: 9 - protein, 726 aa, Artificial Sequence
  Description of Artificial Sequence Synthetic polypeptide
  MOD_RES (165)..(165): Lys or Arg
  MOD_RES (201)..(201): Ala or Ser
  MOD_RES (261)..(261): Ala or Gly
  MOD_RES (306)..(306): Arg or Lys
  MOD_RES (406)..(406): Glu or Gln
  MOD_RES (454)..(454): Thr or Glu
  MOD_RES (486)..(486): Ala or Thr
  MOD_RES (554)..(554): Ser or Asn
  MOD_RES (568)..(568): Gln or Glu
  MOD_RES (579)..(579): Ser or Ala
  MOD_RES (600)..(600): Asn or Asp
AADGYLPDWLEDNLSEGIREWDLKPGAPKPKANQQKQDDGRGLVLPGYYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPAXKRLNFGQTGDSESVPDPQPLGEPPAAPSGVGSNTMXAGGGAPADNNEGADGVGNASGNWHCDSTWLGDRVITTSTRTALPTYNNHLYKQISSQSGXSTNDNTYFGYSTPWGYFDFNRFHCHFSPRDWQRLINNNWGFRPKXLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFXFSYTFEDVPFHSSYAHSQSLDRLNPLIDQYLYYLSRTQTTSGTAGNRXLQFSQAGPSSANQAKNWLPGPCYRQQRVSKTXNQNNNSNFAWTGATKYHLNGRDSLVNPGPAMATHKDDEDKFFPMSGVLIFGKQGAGNSNVDLDNVITXEEEIKTTNPVATEXYGTVATNLQSXNTAPATGTVNSQGALPGVWQXRDVYLQGPIWAKIPHTDGHFHPSPLMGGFGLKHPPPQILIKNTPVPANPPTTFSPAKFASFITQYSTGQVSVEIEELQKENSKRWNPEIQYTSNYNKSTNVDFAVDTNGVYSEPRPIGTRYLTRNL

SEQ ID NO: 10 - DNA, 20 bp, Artificial Sequence
  Description of Artificial Sequence Synthetic primer
caacttcatccacgttcacc

SEQ ID NO: 11 - DNA, 21 bp, Artificial Sequence
  Description of Artificial Sequence Synthetic primer
gttctgagctccaaccattct

SEQ ID NO: 12 - DNA, 21 bp, Artificial Sequence
  Description of Artificial Sequence Synthetic primer
cactgtgggtccttcatcttt

SEQ ID NO: 13 - DNA, 50 bp, Artificial Sequence
  Description of Artificial Sequence Synthetic primer
  modified_base (42)..(42): a, c, t, g, unknown or other
tcgtcggcagcgtcagatgtgtataagagacagcctacgggnggcwgcag

SEQ ID NO: 14 - DNA, 55 bp, Artificial Sequence
  Description of Artificial Sequence Synthetic primer
gtctcgtgggctcggagatgtgtataagagacaggactachvgggtatctaatcc

SEQ ID NO: 15 - DNA, 397 bp, Homo sapiens
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 16 - DNA, 637 bp, Woodchuck hepatitis virus
agtggcggccgctcgagctagcggccgctctagaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactag

SEQ ID NO: 17 - DNA, 320 bp, Homo sapiens
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagg

SEQ ID NO: 18 - DNA, 840 bp, Artificial Sequence
  Description of Artificial Sequence Synthetic polynucleotide
atgaagctgctgggggcgctgctggcactggcggccctactgcagggggccgtgtccctgaagatcgcagccttcaacatcaggacatttgggaggaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtga

SEQ ID NO: 19 - DNA, 849 bp, Artificial Sequence
  Description of Artificial Sequence Synthetic polynucleotide
atgaggggcatgaagctgctgggggcgctgctggcactggcggccctactgcagggggccgtgtccctgaagatcgcagccttcaacatcaggacatttgggaggaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtga

SEQ ID NO: 20 - DNA, 66 bp, Homo sapiens
atgaggggcatgaagctgctgggggcgctgctggcactggcggccctactgcagggggccgtgtcc

SEQ ID NO: 21 - DNA, 783 bp, Artificial Sequence
  Description of Artificial Sequence Synthetic polynucleotide
ctgaagatcgcagccttcaacatcaggacatttgggaggaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtga

SEQ ID NO: 22 - DNA, 846 bp, Homo sapiens
atgaggggcatgaagctgctgggggcgctgctggcactggcggccctactgcagggggccgtgtccctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaag

SEQ ID NO: 23 - DNA, 780 bp, Homo sapiens
ctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaag

SEQ ID NO: 24 - protein, 284 aa, Mus musculus
MRYTGLMGTLLTLVNLLQLAGTLRIAAFNIRTFGETKMSNATLSVYFVKILSRYDIAVIQEVRDSHLVAVGKLLDELNRDKPDTYRYVVSEPLGRKSYKEQYLFVYRPDQVSILDSYQYDDGCEPCGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKI

SEQ ID NO: 25 - protein, 22 aa, Mus musculus
MRYTGLMGTLLTLVNLLQLAGT

SEQ ID NO: 26 - protein, 262 aa, Mus musculus
LRIAAFNIRTFGETKMSNATLSVYFVKILSRYDIAVIQEVRDSHLVAVGKLLDELNRDKPDTYRYVVSEPLGRKSYKEQYLFVYRPDQVSILDSYQYDDGCEPCGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKI

SEQ ID NO: 27 - DNA, 66 bp, Mus musculus
atgcggtacacagggctaatgggaacactgctcaccttggtcaacctgctgcagctggctgggact

SEQ ID NO: 28 - DNA, 789 bp, Mus musculus
ctgagaattgcagccttcaacattcggacttttggggagactaagatgtccaatgctaccctctctgtatactttgtgaaaatcctgagtcgctatgacatcgctgttatccaagaggtcagagactcccacctggttgctgttgggaagctcctggatgaactcaatcgggacaaacctgacacctaccgctatgtagtcagtgagccgctgggccgcaaaagctacaaggaacagtacctttttgtgtacaggcctgaccaggtgtctattctggacagctatcaatatgatgatggctgtgaaccctgtggaaatgacaccttcagcagagagccagccattgttaagttcttttccccatacactgaggtccaagaatttgcgatcgtgcccttgcatgcagccccaacagaagctgtgagtgagatcgacgccctctacgatgtttacctagatgtctggcaaaagtggggcctggaggacatcatgttcatgggagacttcaatgctggctgcagctacgtcacttcctcccagtggtcctccattcgccttcggacaagccccatcttccagtggctgatccctgacagtgcggacaccacagtcacatcaacacactgtgcttatgacaggattgtggttgctggagctctgctccaggctgctgttgttcccaactcggctgttccttttgatttccaagcagaatacggactttccaaccagctggctgaagccatcagtgaccattacccagtggaggtgacactcagaaaaatctga

SEQ ID NO: 29 - DNA, 855 bp, Mus musculus
atgcggtacacagggctaatgggaacactgctcaccttggtcaacctgctgcagctggctgggactctgagaattgcagccttcaacattcggacttttggggagactaagatgtccaatgctaccctctctgtatactttgtgaaaatcctgagtcgctatgacatcgctgttatccaagaggtcagagactcccacctggttgctgttgggaagctcctggatgaactcaatcgggacaaacctgacacctaccgctatgtagtcagtgagccgctgggccgcaaaagctacaaggaacagtacctttttgtgtacaggcctgaccaggtgtctattctggacagctatcaatatgatgatggctgtgaaccctgtggaaatgacaccttcagcagagagccagccattgttaagttcttttccccatacactgaggtccaagaatttgcgatcgtgcccttgcatgcagccccaacagaagctgtgagtgagatcgacgccctctacgatgtttacctagatgtctggcaaaagtggggcctggaggacatcatgttcatgggagacttcaatgctggctgcagctacgtcacttcctcccagtggtcctccattcgccttcggacaagccccatcttccagtggctgatccctgacagtgcggacaccacagtcacatcaacacactgtgcttatgacaggattgtggttgctggagctctgctccaggctgctgttgttcccaactcggctgttccttttgatttccaagcagaatacggactttccaaccagctggctgaagccatcagtgaccattacccagtggaggtgacactcagaaaaatctga

SEQ ID NO: 30 - DNA, 2212 bp, Artificial Sequence
  Description of Artificial Sequence Synthetic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgccgccaccatgaggggcatgaagctgctgggggcgctgctggcactggcggccctactgcagggggccgtgtccctgaagatcgcagccttcaacatcaggacatttgggaggaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtgaagtggcggccgctcgagctagcggccgctctagaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactag

SEQ ID NO: 31 - DNA, 2212 bp, Artificial Sequence
  Description of Artificial Sequence Synthetic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgccgccaccatgaggggcatgaagctgctgggggcgctgctggcactggcggccctactgcagggggccgtgtccctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtgaagtggcggccgctcgagctagcggccgctctagaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactag

SEQ ID NO: 32 - DNA, 849 bp, Homo sapiens
atgaggggcatgaagctgctgggggcgctgctggcactggcggccctactgcagggggccgtgtccctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtga

SEQ ID NO: 33 - DNA, 9 bp, Artificial Sequence
  Description of Artificial Sequence Synthetic oligonucleotide
gccgccacc

SEQ ID NO: 34 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGGSTNDNTYFGYSTPWGYFDFNRFHCHFSPRDWQRLINNNWGFRPKKLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTSGTAGNRTLQFSQAGPSSMANQAKNWLPGPCYRQQRVSKTTNQNNNSNFAWTGATKYHLNGRDSLVNPGPAMATHKDDEDKFFPMSGVLIFGKQGAGNSNVDLDNVMITNEEEIKTTNPVATEEYGTVATNLQSANTAPATGTVNSQGALPGMVWQDRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 35 - protein, 730 aa, Artificial Sequence
  Description of Artificial Sequence Synthetic polypeptide
MAADGYLPDWLEDNLSEGIREWWDLKPGAPKPKANQQKQDDGRGLVLPGYYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGSNTMAAGGGAPADNNEGADGVGNASGNWHCDSTWLGDRVITTSTRTWALPTYNNHLYKQISSQSGGSTNDNTYFGYSTPWGYFDFNRFHCHFSPRDWQRLINNNWGFRPKKLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTSGTAGNRTLQFSQAGPSSANQAKNWLPGPCYRQQRVSKTTNQNNNSNFAWTGATKYHLNGRDSLVNPGPAMATHKDDEDKFFPMSGVLIFGKQGAGNSNVDLDNVITNEEEIKTTNPVATEEYGTVATNLQSANTAPATGTVNSQGALPGVWQDRDVYLQGPIWAKIPHTDGHFHPSPLMGGFGLKHPPPQILIKNTPVPANPPTTFSPAKFASFITQYSTGQVSVEIEELQKENSKRWNPEIQYTSNYNKSTNVDFAVDTNGVYSEPRPIGTRYLTRNL

SEQ ID NO: 36 - DNA, 448 bp, Homo sapiens
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 37 - DNA, 768 bp, Artificial Sequence
  Description of Artificial Sequence Synthetic polynucleotide
gcggccgctctagagagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggt

SEQ ID NO: 38 - DNA, 664 bp, Artificial Sequence
  Description of Artificial Sequence Synthetic polynucleotide
cctctgtctgtctgtctgtctctttggtgtgatgtatgtggtgtgtgtgtattctacaaggttgacatgatgacagaatttaattttcttagcagcaagctcatggatcctggtgataaatgcagcatgactttactgaaaaggctttgtgatcttgaagagtggattgacttcactgtcggcagcacatgcaatctcacttgtttggtgtaatgaaagaagagaatgagaggtggaagggggatggtaatgttgaaaaaaagaatggtacagaggaaactgaggttggagagagatggggtagatggtaagagatggagaaagagggaaggaaatggagagaaagacagagagacagagagagacacacagagagacacacagagacagagaggaagggaaagggaaagagaaaggaagaggaagagggggaggggaaggggaaggggaaggggaagggagagggagaaatgtggacactagccagatttaagggagaaattagggggttgccagtctgtccacctctgatggtggcaactcagcagaaagctgctgggctcagtctggctttgttgagcaaccctgactccaccccttttcttccccacaaagcaagcttttaaagggaaggctttcttcattgaatgactgccacagtacg

SEQ ID NO: 39 - DNA, 1497 bp, Artificial Sequence
  Description of Artificial Sequence Synthetic polynucleotide
gggggtgagcgagggctgctgggaccattgcagggaacaatgataatctaggcttggttcctacccagagagcacgcactcatccttcatgcactcccctgttccaaaccctcactggctccgtactgcctccgaccttccgagactttagcctggctcctgtcaacatctctgacccttactacatgatcctctctttggtccatgctccagcctaatctaattgcggtggcttgtgcgtggtggcattcccagccaccatacctttacccacgctggtccttccatgcggaatgcctttccagggcctgctttgcccgcttctgctcatacacaggcatgccctccaggatggcttcctacctctttcccttgggggattgatctctctgtcttggggttctcggagcccttgacctgacccctttctgtttggcaaaaaagtaatttacctcggtgtccttctccctggtagtctgtgagctccccaaggctgggctgtgcctgattcacctctggaacttgcttagcacagtgcgtggcctgctgcaggtgttcattgagcacttgccgaatgaatgcatgaatgaatgaatgaatgaatgaatgcaaggggctgctaatccacaggactcctcaggtcagccagacgtcccggttccaaggcctgccactgactcacctcaggaccctgcttgaaccattagaactcaccctgcctcactttccccctctgtgaaatggggctccaactcctattcaagctactatcatttgggggcattgtgaggccacagatcccagaacatcagagtcagaggtagcccagaaagcttcccacccatccctacaaatgggaaactgaggtctggagagggaagggcagagttgggctccctgtctcaggctcggacccaccatcaggcctgtctctaaaacgaatcccagctcccacgctgcaccctgagcctggaagcctgagccacacaaggacggggaattttccttcccacttccagaggcctctgaacctccctgagcttgtcccctttggagggtattgggcagcagcgtgggcagaaccccagctcactgtctgggggagcgctgcaggacagccttgtctgtctgtctcagcctgccctggggacccgaggtcagggaggaagtgccgcatctggtcttccccagagcgagagtgtgagcaagggtgggattgcgtgtggcccgagagtagcccctcccctccccctgtccccaccccaaaccctcttaatgaaatcaagctggccctgcggcccagccggggagggaggaaggaggagggacgggaggagggacgggaggagggagggcgggcaggcgccagcccagagcagccccgggcaccagcacggactctctcttccagcccaggtgccccccactctcgctccattcggcgggagcacccagtcctgtacgccaaggaactggtgagtcctggggtcccctcct

SEQ ID NO: 40 - DNA, 1515 bp, Artificial Sequence
  Description of Artificial Sequence Synthetic polynucleotide
acatgcaggcactgtggccccgaaaaactcttcagaggtgagcggctttcaggatgagagggccaacaatggctcatctcctaaagacctcgataaacataatcagtagccccagaaaacacaacagctgtcccaagcctatcccctttgcacctatctcagaaaaggcaacatgcagagtggtcaggagttcaagtcctcagggccctactctgaagctgtgaccctgacaggtcactttagcgctcagcttccctttcctcatctgtaaaatgggaatagaaactgaatctgcttcatggggccttgtaagtattacataagctgtcatatgcaaaatgtccatcacatagttcaccgccccaggcatcctagaaactattaagtcacgataaggaggttgctactgtcctgggacctggggagctcggctgtccagtccgaaagggccttctcgccctcgagaccccttcaagtccctttctctccgggcccgggtcccagagtccccttgtgcttgctcctgagggtgacctgcccgtgccccggggcgagcacaggtgcggccctggccacaatgcgggcgtggcgagccgacacccggggaggccaagggcgcctcgagggagggagggaggaggctgacccgggcgtcccgaaggacccgcccggccccggactccgggcggcagccggcctcgcgccccgcccccgccccgggagccccgcccagtcccgtcgcgccgcgccgccccaccgcccgggttggctgccctggaggccacgcgcggcgatttgccacgcgccgcgtcaccgggccgctcccggcctgggctcccgggggctggtcagggaggtggcggcggctgagcggcggagcgggggcggcagggcgcgacgccgccgggccgccccgccctgggaggcgcccgggccctcatcaagtgaccagtatcccttccaggggaacacggtccttcagaggaaagcgagctccaacccgcggccccggcgccaagccgccgtcatcttcttcctgtgccaggtgatcgtctcctcacccacccggaaaaacatagtccgcccccacgtccttgtggaatagcgcccgcttccaagcaccgtgactctctttgccctacccttgtctttcccattccaattactctagaacccaccgagaggataattcagtcctgaaaagaaactgatggggagaagcgaggaagggaacccaggagggaggggaggcagggctgcggagggacaccgaggcggcggaggtagtgcgcacgcgcagcacagaacgagttccggtctggccgaggcttgtctcctaaaaatagccccggtgtggggatccgtgcgcggatgtcccggcgagtcccgggctgaaagaggcggctccgggcggcgcgaagcgctggtggcgggcccgggctgcggcgtgtgcgcgcccgccagctgctccggagatacggtgagggccgcgggcagcggggctcagtccgcgac

SEQ ID NO: 41 - DNA, 1545 bp, Artificial Sequence
  Description of Artificial Sequence Synthetic polynucleotide
agtgcagcctcaggggttgaaggtgaaatgtgaaatttgctgtaattacttacccaaaatgtctgtattaaaggctattagaagcagttattttaagtacttctttatacatctttccctcttctgtttagtatttaaatgtgcaacaaaagtagtggcagatgttactcatctgaagaaactaaaacaagataagtcagtggcaggagtggtggccggttaaagatctcttcgaagtgctttgttcagtaactgttgttttgtctcacattaagttgtaggtggggtgctgttaaagcttgtccactcacgatggtgttcagctgagacctgtgagttcaagttgtcttgcccagagttaaaggccataaaactaagacagtgtcgttcctgtcttgggtgtaactcctaaaaaactagtcagttcttgcaataggaggaaaattattgcaaataaacctttgatttaccagcttcagtaagttgttcattctgtctctgtagctgtagtttttaccttaacagtttgaggctaccatttctcttcctttcatgcttatggggtccacacaaaccctgctaggctacctacagtataaaatcttaaaactccatgtggtgttgtgagctggtagcagatggactttcttttgcatctgctgtgtacagacttggctttgtactcttttcctgggggagagaaagtctttcttctctgctagttgattcctgtttttgtgaaacagctaagaaaacatctgtggcaggcagaaaaattgacaaaggattgacaaacaccattagctgaaaattttccatttcattgcaactatgcattgtttactttactgtaaatatcttaatacatcatcctctgaatatgctggcagagaagctggagaactgtgatttcaattaaggttagtaattgatggtatctagtgttcaaaggctgaagcttgattaacatctgcttggacaaattgtcattgtgaagtggttttgatgtgcattgaagattatttctttctggcttatctgatgttgtggctgtgaaatgcttgtcttgggtttgcttatttttgtaaactacttcctttggctgtaaattgcagagcactggagctttaccaaaagtgcagtgtataatggtaagcttgtcctaataagggaagcaagaagtgtatttatcacagacatgaaagctaaccgaggacttgagagactcaaactggtgcttttgtctctctctctctgtctttctctctctcacacacacactcacacactcacacacatgcacacacacacatacacacacacaaaaatgaagcacttactttagaaagattatggtaagcatgctggctcagtcttgaacctttgtcacccctcacgttgcacaccaaagacataccctagtgattaaatgctgattttgtgtacgattgtccacggacgccaaaacaatcacagagctgcttgatttgttttaattaccagcacaaaatgccatcagtctgggacgtgatcgggcagaggtgt

SEQ ID NO: 42 - DNA, 2030 bp, Artificial Sequence
  Description of Artificial Sequence Synthetic polynucleotide
atgaggggcatgaagctgctgggggcgctgctggcactggcggccctactgcagggggccgtgtccctgaagatcgcagccttcaacatcaggacatttgggaggaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtgaagtggcggccgctcgagctagcggccgctctagaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactagaagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccga

SEQ ID NO: 43 - DNA, 2030 bp, Artificial Sequence
  Description of Artificial Sequence Synthetic polynucleotide
atgaggggcatgaagctgctgggggcgctgctggcactggcggccctactgcagggggccgtgtccctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtgaagtggcggccgctcgagctagcggccgctctagaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactagaagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccga

SEQ ID NO: 44 - DNA, 849 bp, Artificial Sequence; identical to SEQ ID NO: 19
  Description of Artificial Sequence Synthetic polynucleotide

SEQ ID NO: 45 - protein, 724 aa, Adeno-associated virus
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 46 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 47 - protein, 736 aa, Adeno-associated virus
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 48 - DNA, 2211 bp, Adeno-associated virus
atggctgctgacggttatcttccagattggctcgaggacaacctttctgaaggcattcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccccattggcacccgttaccttacccgtcccctgtaa

SEQ ID NO: 49 - protein, 737 aa, Adeno-associated virus
MAADGYLPDWLEDTLSEGIRQWWKLKPGPPPPKPAERHKDDSRGLVLPGYKYLGPGNGLDKGEPVNAADAAALEHDKAYDQQLKAGDNPYLRYNHADAEFQERLQEDTSFGGNLGRAVFQAKKRVLEPLGLVEEPVKTAPGKKRPVEHSPVEPDSSSGTGKAGQQPARKRLNFGQTGDTDSAADPQPLGEPPAAPSGLGTGTMAAGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTQNEGTKTIANNLTSTVQVFTDSEYQLPYVLGSAHQGCLPPFPADVFMIPQYGYLTLNNGSQAMGRSSFYCLEYFPSQMLRTGNNFQFTYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQILIKNTPVPADPPTAFNKDKLNSFITQYSTGQVSVEIEWELQKENSKRWNPEIQYTSNYYKSTSVDFAVNTEGVYSEPRPIGTRYLTRNL

SEQ ID NO: 50 - DNA, 2214 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttccgggttacaaatacctcggacccggcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaacctgttaagacggctccgggaaaaaagaggccggtagagcactctcctgtggagccagactcctcctcgggaaccggaaaggcgggccagcagcctgcgaggaagcgactcaactttggtcagactggagacaccgactccgccgctgacccccagcctctcggagaaccaccagcagccccctctggtctgggaactggtacaatggctgcaggcggtggcgctccaatggcagacaataacgaaggcgccgacggagtgggtaatgcctcgggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtactttgacttcaaccgcttccactgccacttctcaccacgtgactggcaaagactcatcaacaacaactggggattccggcccaagagactcaacttcaagctcttcaacatccaggtcaaggaggtcacgcagaatgaaggcaccaagaccatcgccaataaccttaccagcacggttcaggtgtttactgactcggagtaccagctgccgtacgttctcggctctgcccaccagggctgcctgcctccgttcccggcggacgtgttcatgattccgcagtacggctacctaacgctcaacaatggcagccaggcgatgggtcgctcgtccttctactgcctggagtactttccgtcgcagatgctgagaaccggcaacaacttccagtttacttacaccttcgaggacgtgcctttccacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggcggctttggcctgaaacatcctccgcctcagatcctgatcaagaacacgcctgtacctgcggatcctccaacggccttcaacaaggacaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 51 - protein, 305 aa, Homo sapiens
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 52 - DNA, 915 bp, Homo sapiens
atgtcacgggagctggccccactgctgcttctcctcctctccatccacagcgccctggccatgaggatctgctccttcaacgtcaggtcctttggggaaagcaagcaggaagacaagaatgccatggatgtcattgtgaaggtcatcaaacgctgtgacatcatactcgtgatggaaatcaaggacagcaacaacaggatctgccccatactgatggagaagctgaacagaaattcaaggagaggcataacgtacaactatgtgattagctctcggcttggaagaaacacatataaagaacaatatgcctttctctacaaggaaaagctggtgtctgtgaagaggagttatcactaccatgactatcaggatggagacgcagatgtgttttccagggagccctttgtggtctggttccaatctccccacactgctgtcaaagacttcgtgattatccccctgcacaccaccccagagacatccgttaaggagatcgatgagttggttgaggtctacacggacgtgaaacaccgctggaaggcggagaatttcattttcatgggtgacttcaatgccggctgcagctacgtccccaagaaggcctggaagaacatccgcttgaggactgaccccaggtttgtttggctgatcggggaccaagaggacaccacggtgaagaagagcaccaactgtgcatatgacaggattgtgcttagaggacaagaaatcgtcagttctgttgttcccaagtcaaacagtgtttttgacttccagaaagcttacaagctgactgaagaggaggccctggatgtcagcgaccactttccagttgaatttaaactacagtcttcaagggccttcaccaacagcaaaaaatctgtcactctaaggaagaaaacaaagagcaaacgctcc

SEQ ID NO: 53 - protein, 285 aa, Homo sapiens
MRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 54 - protein, 5 aa, Adeno-associated virus
NGTSG

SEQ ID NO: 55 - protein, 4 aa, Artificial Sequence
  Description of Artificial Sequence Synthetic peptide
SGTH

SEQ ID NO: 56 - protein, 4 aa, Artificial Sequence
  Description of Artificial Sequence Synthetic peptide
SDTH

SEQ ID NO: 57 - protein, 5 aa, Adeno-associated virus
GGTAN

SEQ ID NO: 58 - protein, 5 aa, Artificial Sequence
  Description of Artificial Sequence Synthetic peptide
DGSGL

SEQ ID NO: 59 - protein, 9 aa, Artificial Sequence
  Description of Artificial Sequence Synthetic peptide
CILDLPKFC

SEQ ID NO: 60 - protein, 4 aa, Artificial Sequence
  Description of Artificial Sequence Synthetic peptide
GGGS